(19) United States
(12) Patent Application Publication
(10) Pub. No.: US 2017/0269092 A1
(43) Pub. Date: Sep. 21, 2017

(54) ANTI-MUTANT CALRETICULIN ANTIBODIES AND THEIR USE IN THE DIAGNOSIS AND THERAPY OF MYELOID MALIGNANCIES

(71) Applicant: CEMM - Forschungszentrum Fuer Molekulare Medizin GmbH, Wien (AT)

(72) Inventor: Robert Kralovics, Wien (AT)

(73) Assignee: CEMM - Forschungszentrum Fuer Molekulare Medizin GMBH, Wien (AT)

(21) Appl. No.: 15/532,453

(22) PCT Filed: Dec. 2, 2015
(86) PCT No.: PCT/EP2015/078361

§ 371 Date: Jun. 1, 2017

(30) Foreign Application Priority Data
Dec. 2, 2014 (EP) 14195928.8

(51) Int. Cl. G01N 33/574; G01N 33/543; G01N 33/53; A61K 39/395; C07K 16/28

(52) U.S. Cl. CPC G01N 33/57492; A61K 39/395; C07K 16/28; G01N 33/53; G01N 33/543; C07K 2317/76; G01N 2333/70596; G01N 2800/52

(57) ABSTRACT

The present invention relates to an antibody that specifically binds to a mutant calreticulin protein, wherein the variable region of the heavy chain of said antibody comprises a CDR-H3 region having an amino acid sequence as depicted in SEQ ID NO.: 3, or a CDR sequence having 75% or more amino acid identity to said CDR; or wherein the variable region of the heavy chain of said antibody comprises a CDR-H3 region having an amino acid sequence as depicted in SEQ ID NO.: 6, or a CDR sequence having 75% or more amino acid identity to said CDR. Hybridoma 8B2-H6-10.7 deposited under accession number DSM ACC3249 with the depositary institute DSMZ on Sep. 12, 2014 as well as antibodies obtainable therefrom are subject of the present invention. The antibodies provided herein can be used in the diagnosis of or therapeutic intervention in myeloid malignancies.

The present invention relates to an antibody that specifically binds to a mutant calreticulin protein, wherein the variable region of the heavy chain of said antibody comprises a CDR-H3 region having an amino acid sequence as depicted in SEQ ID NO.: 3, or a CDR sequence having 75% or more amino acid identity to said CDR; or wherein the variable region of the heavy chain of said antibody comprises a CDR-H3 region having an amino acid sequence as depicted in SEQ ID NO.: 6, or a CDR sequence having 75% or more amino acid identity to said CDR. Hybridoma 8B2-H6-10.7 deposited under accession number DSM ACC3249 with the depositary institute DSMZ on Sep. 12, 2014 as well as antibodies obtainable therefrom are subject of the present invention. The antibodies provided herein can be used in the diagnosis of or therapeutic intervention in myeloid malignancies.

Primary myelofibrosis (PMF), essential thrombocythemia (ET) and polycythemia vera (PV) are monoclonal hematological disorders that belong to the classical BCR-ABL negative myeloproliferative neoplasms (MPN) (Campbell & Green, 2006). Since the 2005 discovery of a somatic mutation in the JAK2 kinase gene, a tremendous progress has been made in molecular diagnosis, clinical management, treatment and molecular understanding of MPN. The valine to phenylalanine (V617F) mutation constitutively activates the Jak2 kinase resulting in increased phosphorylation of its substrates (Stat5, Stat3, Erk, etc.) and leading to increased cytokine responsiveness of myeloid cells (Baxter et al, 2005; James et al, 2005; Kralovics et al, 2005; Levine et al, 2005). Identification of additional mutations soon followed such as in JAK2 exon 12 in PV (Scott et al, 2007) and in the thrombopoietin receptor gene MPL in PMF and ET (Pardanani et al, 2006; Pikman et al, 2006). Although the three MPN disease entities differ in their clinical presentation, they share many molecular as well as clinical features. The JAK2-V617F mutation is present in about 95% of PV cases, 60% PMF and 50% of ET cases, respectively. Mutations in JAK2 exon 12 are specific to about 3% of PV cases whereas MPL mutations are restricted to the PMF (5%) and ET (3%). All three MPN entities are predisposed at a variable degree to thrombosis, bleeding and leukemic transformation (Sverdlow et al, 2008). Although patients may remain in the chronic phase of MPN for several years, disease progression occurs in a form of secondary myelofibrosis in PV and ET, development of accelerated phase with variable degree of pancytopenia followed by leukemic transformation affecting all three MPN entities (Sverdlow et al, 2008).

Somatic mutations accumulate during the entire clonal evolution of MPN hematopoietic stem cells. These acquired genetic alterations may be point mutations, chromosomal lesions and epigenetic defects and they all may contribute to the fitness of the evolving clone (Klampfl et al, 2011; Kralovics, 2008). These mutations may accelerate proliferation by various means, decrease differentiation potential of progenitors or render them less susceptible to apoptosis. Mutations affecting these mechanisms have been described in genes such as TET2 (Delhommeau et al, 2009), EZH2 (Ernst et al, 2010), DNMT3A (Stegelmann et al, 2011), ASXL1 (Stein et al, 2011), and TP53 (Harutyunyan et al, 2011) in different types of myeloid malignancies including MPN (Milosevic & Kralovics, 2013). However, so far only JAK2 and MPL mutations are considered strongly MPN associated and they represent the most useful molecular markers of MPN.

Despite the progress made in the understanding of the molecular pathogenesis of MPN approximately half of the patients with PMF and ET lack a molecular marker for diagnosis as these patients are negative for both JAK2 and MPL mutations. Recently, mutant calreticulin proteins has been identified and found to be associated with PMF and ET; see, inter alia, Klampfl et al. (N Engl J Med 2013; 369:2379-2390 Dec. 19, 2013), Nangalia et al. (N Engl J Med 2013; 369:2391-2405) and Cazzola and Kralovics (Blood 2014; 123(24):3714-9).

The technical problem underlying the present invention is the provision of specific antibodies that specifically bind to a mutant calreticulin protein and their use in the diagnosis and therapy of myeloid malignancies.

The technical problem is solved by provision of the embodiments characterized in the claims.

Accordingly, the present invention relates to an antibody that specifically binds to a mutant calreticulin protein,

wherein the variable region of the heavy chain of said antibody comprises a CDR-H3 region having an amino acid sequence as depicted in SEQ ID NO.: 3, or a CDR sequence having 75% or more amino acid identity to said CDR;
or
wherein the variable region of the heavy chain of said antibody comprises a CDR-H3 region having an amino acid sequence as depicted in SEQ ID NO.: 6, or a CDR sequence having 75% or more amino acid identity to said CDR.

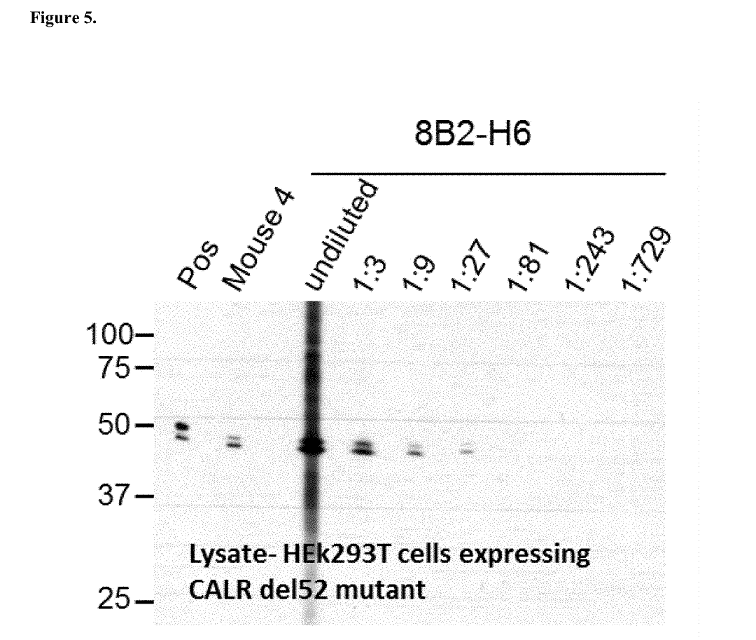

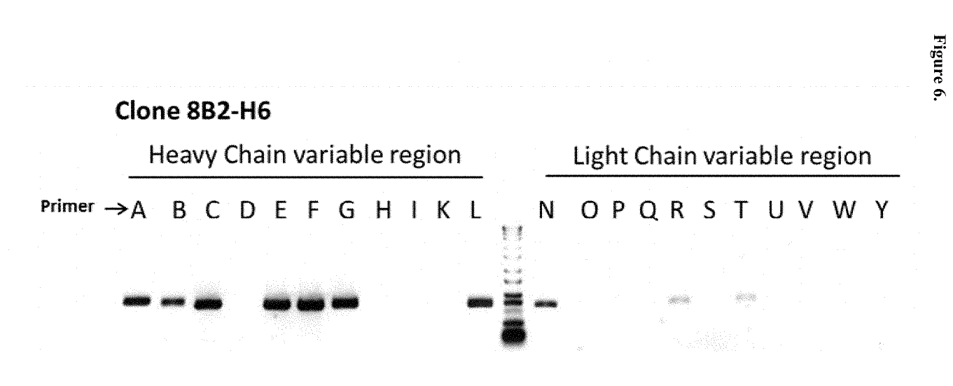

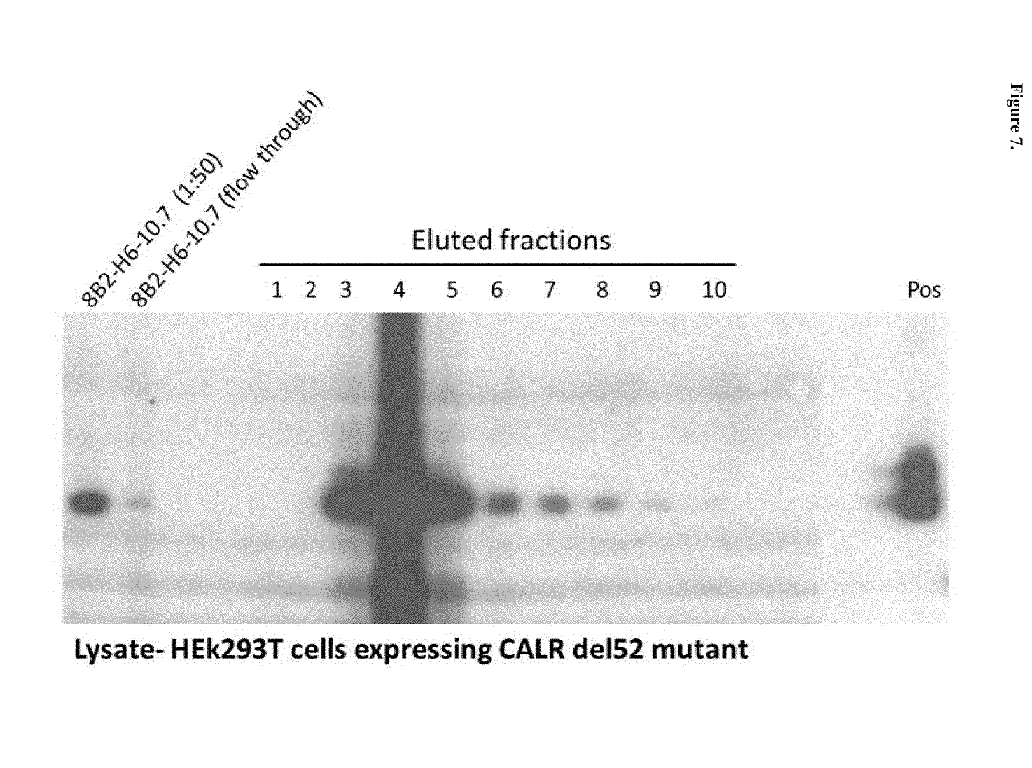

It is shown herein that a specific antibody 8B2-H6 was generated using a synthetic peptide having the c-terminal end sequence of the mutant calreticulin protein (RRKMSPARPRTSCREACLQGWTEA); see Example 1 and FIGS. 4 and 5. Antibody 8B2-H6 detected the CALR del52 mutant (FIGS. 4 and 5). Anti-wild-type calreticulin antibody (Millipore MABT145) was used as positive control (Pos). MABT145 recognizes all three forms of calreticulin—wild type, mutant del 52 and deleted exon 9 and is therefore not specifically binding to mutant calreticulin protein.

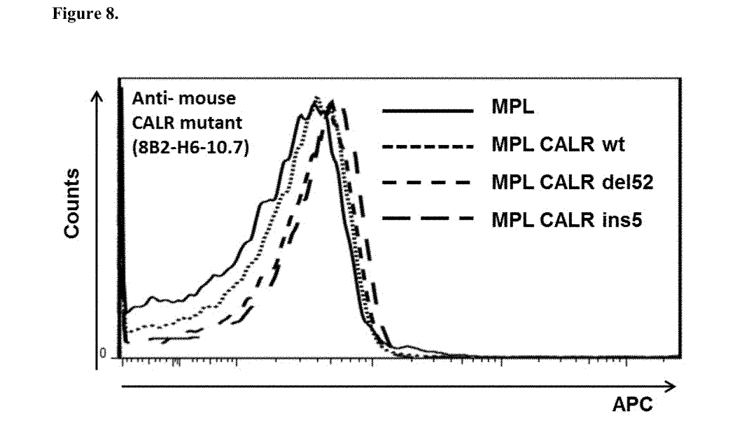

The RNA from clone 8B2-H6 was extracted and cDNA was prepared. Primers from the Mouse IgG Library primer set (Progen) were used to amplify the variable regions of the specific immunoglobulin heavy chain and light chain produced by this clone (FIG. 6) and the PCR product was sequenced. A specific clone, 8B2-H6-10.7, was used to stain Ba/F3-MPL cells expressing the different CALR constructs for detection of the surface CALR by FACS analysis. Anti-mouse PE antibody was used as secondary antibody. FIG. 8 shows specific detection of mutant CALR proteins, both del52 (Type1) and ins5 (Type2), on the surface of the respective Ba/F3 cells. Thus, it is demonstrated herein that the antibody obtained from hybridoma 8B2-H6-10.7 binds indeed specifically to mutant calreticulin, in particular in vivo situations, but not to wild-type calreticulin.

The hybridoma clone 8B2-H6-10.7 has been deposited under accession number DSM ACC3249 with the depositary institute DSMZ on Sep. 12, 2014.

The terms “antibody that specifically binds to a mutant calreticulin protein”, “antibody that specifically binds to a mutant CALR protein”, “anti-mutant calreticulin protein antibody”, “anti-mutant CALR protein antibody”, “antibody to mutant calreticulin protein”, “antibody to mutant CALR protein”, “mutant calreticulin protein antibody”, “mutant CALR protein antibody” and the like are used interchangeably herein. These terms refer to an antibody that specifically binds to a mutant CALR protein according to the invention. The term “antibody” is not limited to full antibodies (immunoglobulins), like murine antibodies (e.g. IgG2a immunoglobulin), or chimeric antibodies, or cross-cloned antibodies, or CDR-grafted antibodies, or humanized antibodies or (fully) human antibodies (e.g. IgA, IgD, IgE, IgG or IgM immunoglobulins). The term “antibody” encompasses a functional fragment of the antibody or a functional derivative thereof. The term “antibody” also comprises, inter alia, antibody fragments (such as a F(ab)-fragment or a F(ab)²-fragments), artificial/synthetic antibodies, antibody derivatives, single chain antibodies (like bispecific single chain antibodies), diabodies, triabodies, a bivalent antibody-construct. The term “antibody” also relates to binding molecules that comprise CDRs or binding portions of the antibodies described herein.

Wild-type calreticulin (CALR) has a C-terminal 4 amino acids sequence (KDEL) containing the endoplasmatic reticulum (ER) retention signal. Hence, wild-type calreticulin is primarily localized in the ER. When localized to the ER, calreticulin has, as a multi-functional chaperone protein, important functions in directing proper conformation of proteins and glycoproteins as well as in homeostatic control of cytosolic and ER calcium levels; see Jiang (2014) Membranes 4(3), 630-641. Yet, wild-type calreticulin (CALR) has also been found to be localized to the cell surface and the extracellular matrix; Jiang (2014; loc. cit.), Gold (2010) FASEB 24, 665-683; Wang (2012) Int J Biochem Cell Biol 44(6):842-6; Cho (2010) Sci Transl Med 2(63):63ra94; Gardai (2005) Cell 123(2), 321-34. These studies propose the following mechanisms of wild-type calreticulin when localized outside the ER, in particular at the cell surface:
Destabilization of cell-surface proteins and/or inhibition of cell surface expression of proteins (like inhibition of cell surface expression of cystic fibrosis transmembrane conductance regulator (CFTR) which may be due to co-internalization of cell surface calreticulin and CFTR
Cell adhesion
Focal adhesion disassembly (e.g. regulation of focal adhesions via TSP1)
Cell migration and homing cells to sites of injury/repair, such as cutaneous wound healing
Anoikis
Phagocytosis (calreticulin is described as a pro-phagocytic signal that is counterbalanced by CD47)

Though wild-type calreticulin does not have a transmembrane region, it is thought to be involved in signaling, e.g. via binding to or engaging by binding or modifying other transmembrane molecules on the cell surface to mediate signaling; see Gold (loc. cit). Wild-type calreticulin is also secreted into the serum and has been localized to the extracellular matrix (ECM); a role in enhancing ECM formation and fibroblast anoikis resistance has been proposed in this context; see Gold (loc. cit).

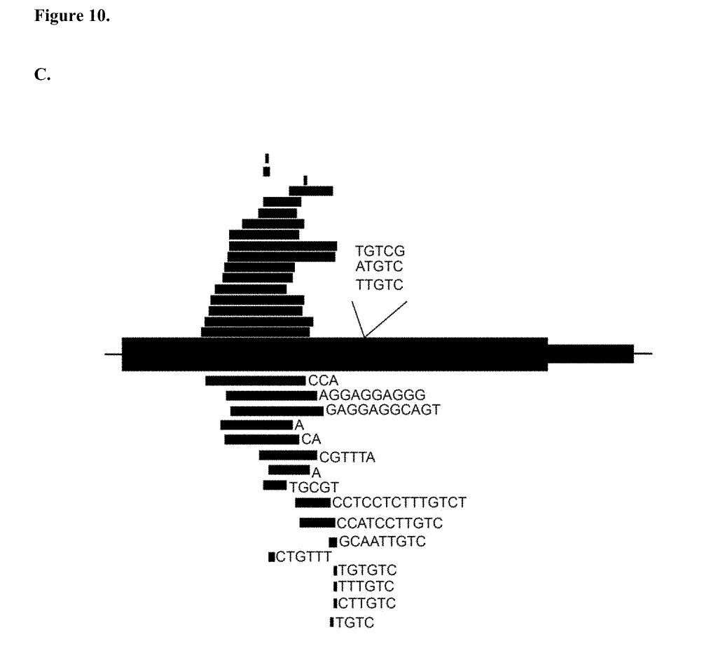

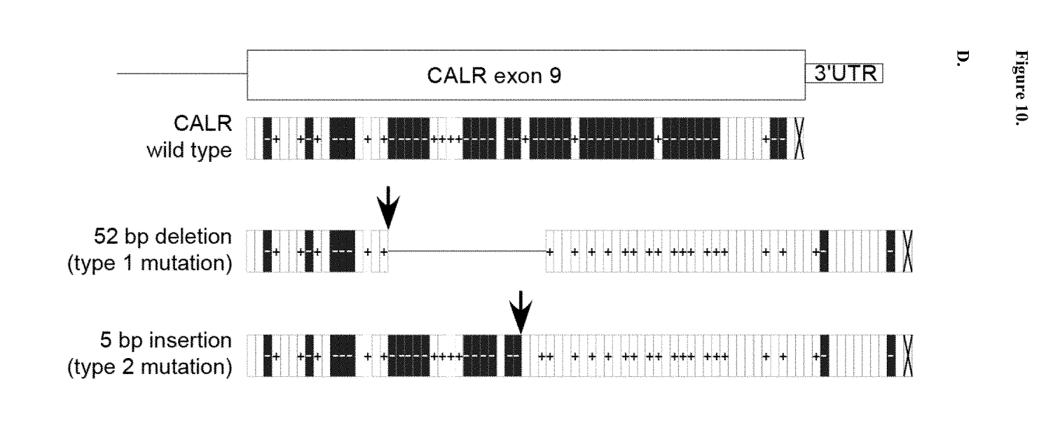

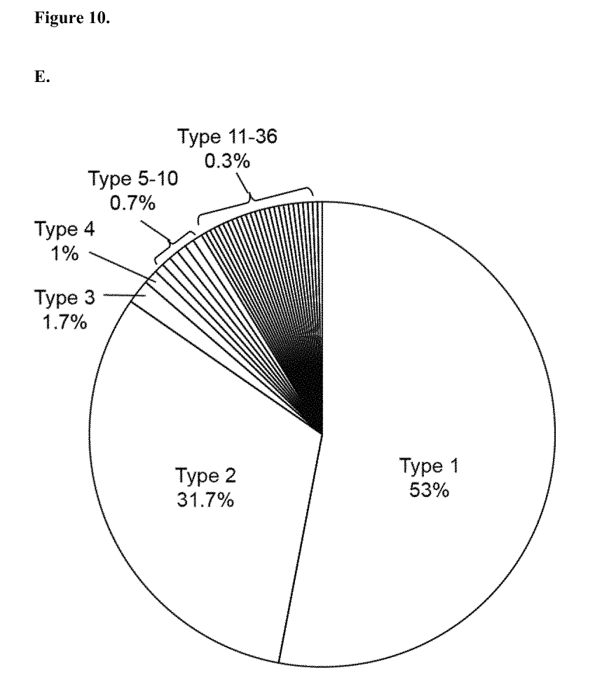

Mutant calreticulin proteins have been identified and found to be associated with myeloid malignancies, like PMF and ET; see, inter alia, Klampfl et al. (N Engl J Med 2013; 369:2379-2390 Dec. 19, 2013) and Nangalia et al. (N Engl J Med 2013; 369:2391-2405; EP 14 18 4835.8; PCT/EP2014/069638 and U.S. application Ser. No. 14/486,973) Mutant calreticulin has a frameshift in exon 9 of the coding sequence of wild-type calreticulin. This frameshift results in the replacement of the C-terminal negatively charged amino acids (aspartic and glutamic acid rich) of wild-type calreticulin by a predominantly positively charged polypeptide rich in arginine and methionine; see FIG. 10. As the negatively charged C-terminus of calreticulin is a low affinity high capacity Ca2+ binding domain, the Ca2+ binding function of the mutant protein is probably lost.

The predominant mutations of CALR are type 1 (“CALR del52 mutation) and type 2 mutations (see Table below and FIG. 10). These mutants and their use in accordance with the present invention is therefore preferred. The following Table shows exemplary C-terminal amino acid residues/sequences of mutant calreticulin proteins to which the antibodies provided herein can specifically bind.

TABLE
C-terminal amino acid sequences of insertion/deletion frameshift
mutations of CALR found in MPN patients. The Table discloses
SEQ ID NOs 35 to 70, respectively, in order of appearance.

| | |
|---|---|
| Type 1 | TRRMMRTKMRMRRMRRTRRIKMRRKMSPARPRTSCREACLQGWTEA- |
| Type 2 | NCRRMMRTKMRMRRMRRTRRKMRRKMSPARPRTSCREACLQGWTEA- |
| Type 3 | QRTRRMMRTKMRMRRMRRTRRKMRRKMSPARPRTSCREACLQGWTEA- |
| Type 4 | RRRQRTRRMMRTKMRMRRMRRTRRKMRRKMSPARPRTSCREACLQGWTEA- |
| Type 5 | GQRTRRMMRTKMRMRRMRRTRRKMRRKMSPARPRTSCREACLQGWTEA- |
| Type 6 | RRQRTRRMMRTKMRMRRMRRTRRKMRRKMSPARPRTSCREACLQGWTEA- |
| Type 7 | RRMMRTKMRMRRMRRTRRKMRRKMSPARPRTSCREACLQGWTEA- |
| Type 8 | RRQRTRRMMRTKMRMRRMRRTRRKMRRKMSPARPRTSCREACLQGWTEA- |
| Type 9 | RQRTRRMMRTKMRMRRMRRTRRKMRRKMSPARPRTSCREACLQGWTEA- |
| Type 10 | MCRRMMRTKMRMRRMRRTRRKMRRKMSPARPRTSCREACLQGWTEA- |
| Type 11 | DQRQRTRRMMRTKMRMRRMRRTRRKMRRKMSPARPRTSCREACLQGWTEA- |
| Type 12 | RRRRQRTRRMMRTKMRMRRMRRTRRKMRRKMSPARPRTSCREACLQGWTEA- |
| Type 13 | QRRRQRTRRMMRTKMRMRRMRRTRRKMRRKMSPARPRTSCREACLQGWTEA- |
| Type 14 | RRRQRTRRMMRTKMRMRRMRRTRRKMRRKMSPARPRTSCREACLQGWTEA- |
| Type 15 | RRRERTRRMMRTKMRMRRMRRTRRKMRRKMSPARPRTSCREACLQGWTEA- |
| Type 16 | QRRQRTRRMMRTKMRMRRMRRTRRKMRRKMSPARPRTSCREACLQGWTEA- |
| Type 17 | RRQWTRRMMRTKMRMRRMRRTRRKMRRKMSPARPRTSCREACLQGWTEA- |
| Type 18 | RMMRTKMRMRRMRRTRRKMRRKMSPARPRTSCREACLQGWTEA- |
| Type 19 | RQRTRRMMRTKMRMRRMRRTRRKMRRKMSPARPRTSCREACLQGWTEA- |
| Type 20 | GRRQRTRRMMRTKMRMRRMRRTRRKMRRKMSPARPRTSCREACLQGWTEA- |
| Type 21 | AFKRTRRMMRTKMRMRRMRRTRRKMRRKMSPARPRTSCREACLQGWTEA- |
| Type 22 | NAKRRRRQRTRRMMRTKMRMRRMRRTRRKMRRKMSPARPRTSCREACLQGWTEA- |
| Type 23 | CVRRRRQRTRRMMRTKMRMRRMRRTRRKMRRKMSPARPRTSCREACLQGWTEA- |
| Type 24 | RRQRTRRMMRTKMRMRRMRRTRRKMRRKMSPARPRTSCREACLQGWTEA- |
| Type 25 | RQRTRRMMRTKMRMRRMRRTRRKMRRKMSPARPRTSCREACLQGWTEA- |
| Type 26 | NAKRRRRQRTRRMMRTKMRMRRMRRTRRKMRRKMSPARPRTSCREACLQGWTEA- |
| Type 27 | CFAKRRRRQRTRRMMRTKMRMRRMRRTRRKMRRKMSPARPRTSCREACLQGWTEA- |
| Type 28 | RRMMRTKMRMRRMRRTRRKMRRKMSPARPRTSCREACLQGWTEA- |
| Type 29 | PPLCLRRMMRTKMRMRRMRRTRRKMRRKMSPARPRTSCREACLQGWTEA- |
| Type 30 | DHPCRRMMRTKMRMRRMRRTRRKMRRKMSPARPRTSCREACLQGWTEA- |
| Type 31 | GNCRRMMRTKMRMRRMRRTRRKMRRKMSPARPRTSCREACLQGWTEA- |
| Type 32 | CRRMMRTKMRMRRMRRTRRKMRRKMSPARPRTSCREACLQGWTEA- |
| Type 33 | CRRMMRTKMRMRRMRRTRRKMRRKMSPARPRTSCREACLQGWTEA- |
| Type 34 | TCRRMMRTKMRMRRMRRTRRKMRRKMSPARPRTSCREACLQGWTEA- |
| Type 35 | ICRRMMRTKMRMRRMRRTRRKMRRKMSPARPRTSCREACLQGWTEA- |
| Type 36 | CRRMMRTKMRMRRMRRTRRKMRRKMSPARPRTSCREACLQGWTEA- |

Preferably, the herein provided antibodies specifically bind to the C-terminus of mutant calreticulin (or fragment or epitope thereof), for example, to one or more of the sequences shown in SEQ ID NO: 35 to 70.

| | |
|---|---|
| SEQ ID NO: 35 | TRRMMRTKMRMRRMRRTRRKMRRKMSPARPRTSCREACLQGWTEA- |
| SEQ ID NO: 36 | NCRRMMRTKMRMRRMRRTRRKMRRKMSPARPRTSCREACLQGWTEA- |
| SEQ ID NO: 37 | QRTRRMMRTKMRMRRMRRTRRKMRRKMSPARPRTSCREACLQGWTEA- |
| SEQ ID NO: 38 | RRRQRTRRMMRTKMRMRRMRRTRRKMRRKMSPARPRTSCREACLQGWTEA- |
| SEQ ID NO: 39 | GQRTRRMMRTKMRMRRMRRTRRKMRRKMSPARPRTSCREACLQGWTEA- |
| SEQ ID NO: 40 | RRQRTRRMMRTKMRMRRMRRTRRKMRRKMSPARPRTSCREACLQGWTEA- |
| SEQ ID NO: 41 | RRMMRTKMRMRRMRRTRRKMRRKMSPARPRTSCREACLQGWTEA- |
| SEQ ID NO: 42 | RRQRTRRMMRTKMRMRRMRRTRRKMRRKMSPARPRTSCREACLQGWTEA- |
| SEQ ID NO: 43 | RQRTRRMMRTKMRMRRMRRTRRKMRRKMSPARPRTSCREACLQGWTEA- |
| SEQ ID NO: 44 | MCRRMMRTKMRMRRMRRTRRKMRRKMSPARPRTSCREACLQGWTEA- |
| SEQ ID NO: 45 | DQRQRTRRMMRTKMRMRRMRRTRRKMRRKMSPARPRTSCREACLQGWTEA- |
| SEQ ID NO: 46 | RRRRQRTRRMMRTKMRMRRMRRTRRKMRRKMSPARPRTSCREACLQGWTEA- |
| SEQ ID NO: 47 | QRRRQRTRRMMRTKMRMRRMRRTRRKMRRKMSPARPRTSCREACLQGWTEA- |
| SEQ ID NO: 48 | RRRQRTRRMMRTKMRMRRMRRTRRKMRRKMSPARPRTSCREACLQGWTEA- |
| SEQ ID NO: 49 | RRRERTRRMMRTKMRMRRMRRTRRKMRRKMSPARPRTSCREACLQGWTEA- |
| SEQ ID NO: 50 | QRRQRTRRMMRTKMRMRRMRRTRRKMRRKMSPARPRTSCREACLQGWTEA- |
| SEQ ID NO: 51 | RRQWTRRMMRTKMRMRRMRRTRRKMRRKMSPARPRTSCREACLQGWTEA- |
| SEQ ID NO: 52 | RMMRTKMRMRRMRRTRRKMRRKMSPARPRTSCREACLQGWTEA- |
| SEQ ID NO: 53 | RQRTRRMMRTKMRMRRMRRTRRKMRRKMSPARPRTSCREACLQGWTEA- |
| SEQ ID NO: 54 | GRRQRTRRMMRTKMRMRRMRRTRRKMRRKMSPARPRTSCREACLQGWTEA- |
| SEQ ID NO: 55 | AFKRTRRMMRTKMRMRRMRRTRRKMRRKMSPARPRTSCREACLQGWTEA- |
| SEQ ID NO: 56 | NAKRRRRQRTRRMMRTKMRMRRMRRTRRKMRRKMSPARPRTSCREACLQGWTE |
| SEQ ID NO: 57 | CVRRRRQRTRRMMRTKMRMRRMRRTRRKMRRKMSPARPRTSCREACLQGWTEA |
| SEQ ID NO: 58 | RRQRTRRMMRTKMRMRRMRRTRRKMRRKMSPARPRTSCREACLQGWTEA- |
| SEQ ID NO: 59 | RQRTRRMMRTKMRMRRMRRTRRKMRRKMSPARPRTSCREACLQGWTEA- |
| SEQ ID NO: 60 | NAKRRRRQRTRRMMRTKMRMRRMRRTRRKMRRKMSPARPRTSCREACLQGWTE |
| SEQ ID NO: 61 | CFAKRRRRQRTRRMMRTKMRMRRMRRTRRKMRRKMSPARPRTSCREACLQGWT |
| SEQ ID NO: 62 | RRMMRTKMRMRRMRRTRRKMRRKMSPARPRTSCREACLQGWTEA- |
| SEQ ID NO: 63 | PPLCLRRMMRTKMRMRRMRRTRRKMRRKMSPARPRTSCREACLQGWTEA- |
| SEQ ID NO: 64 | DHPCRRMMRTKMRMRRMRRTRRKMRRKMSPARPRTSCREACLQGWTEA- |
| SEQ ID NO: 65 | GNCRRMMRTKMRMRRMRRTRRKMRRKMSPARPRTSCREACLQGWTEA- |
| SEQ ID NO: 66 | CRRMMRTKMRMRRMRRTRRKMRRKMSPARPRTSCREACLQGWTEA- |
| SEQ ID NO: 67 | CRRMMRTKMRMRRMRRTRRKMRRKMSPARPRTSCREACLQGWTEA- |
| SEQ ID NO: 68 | TCRRMMRTKMRMRRMRRTRRKMRRKMSPARPRTSCREACLQGWTEA- |
| SEQ ID NO: 69 | ICRRMMRTKMRMRRMRRTRRKMRRKMSPARPRTSCREACLQGWTEA- |
| SEQ ID NO: 70 | CRRMMRTKMRMRRMRRTRRKMRRKMSPARPRTSCREACLQGWTEA- |

It is envisaged herein that the herein provided antibodies can specifically bind to a fragment or part of the C-terminus of mutant calreticulin protein. It is preferred that the herein provided antibodies specifically bind to RRKMSPARPRTSCREACLQGWTEA (SEQ ID NO: 71).

The last 4 amino acids of wild-type calreticulin (KDEL) containing the endoplasmatic reticulum retention signal is absent in the mutant calreticulin. This suggests that the mutant protein is less represented in the ER compared to the wild type protein.

In view of the altered C-terminus of mutant calreticulin and the absent KDEL sequence it was not clear whether mutant calreticulin would have similar biological activities as wild-type calreticulin. For example, it was not known whether mutant calreticulin would be present on the cell surface.

In the present application it was surprisingly shown that the herein provided antibody was able to specifically bind to mutant calreticulin in an FACS assay using mutant calreticulin expressing cells; see Example 1 and FIG. 8. This indicates that mutant calreticulin protein is localized on the cell surface/present on the extracellular side of the plasma membrane/localized at the extracellular side of a plasma membrane. Thus, mutant calreticulin protein can be involved in the same regulatory mechanisms as wild-type calreticulin.

Due to its presence on the cellular surface, mutant calreticulin can be used as a cell surface marker using e.g. cells expressing mutant calreticulin and/or patient samples containing whole/living cells (like blood samples, serum samples or bone marrow samples). For example, patient samples containing whole/living cells can be used in the diagnosis of myeloid malignancies, like for example in the diagnosis of meyloproliferative neoplasms like primary myelofibrosis (PMF) or essential thrombocythemia (ET) or in the diagnosis of a myelodysplastic syndrome, like refractory anemia with ringed sideroblasts and thrombocythemia (RARS-T) using the herein provided antibodies. For example, flow cytometry techniques, like fluorescence-activated cell sorting (FACS) assays, can be used in this analysis. The use of the herein provided antibodies in such assays allows are more convenient and/or quicker analysis compared to Western Blot or ELISA techniques. As a further advantage, such assays require less patient material.

The terms “specifically binding to a mutant calreticulin protein” and “capable of specifically binding to a mutant calreticulin protein” are used interchangeably herein. The term “specifically binding to a mutant calreticulin protein” refers particularly to the capacity of the herein provided antibodies to “specifically bind to the C-terminal part of mutant calreticulin protein”, preferably to the C-terminal part of mutant calreticulin protein as defined herein and/or shown in the above table (or to a fragment thereof). It is envisaged herein that the herein provided antibodies can specifically bind to fragments or derivatives of the mutant calreticulin proteins as defined herein, for example also to polypeptides having at least 70% or more identity to herein provided mutant calreticulin protein(s), in particular to the C-terminal part of mutant calreticulin protein as defined herein and/or shown in the above table.

Within the scope of this invention are antibodies having the capacity to specifically bind to mutant calreticulin protein(s). In a certain aspect, antibodies provided or to be used in accordance with the present invention, bind to the same epitope(s) as any of the antibodies that can be obtained or that are obtainable from hybridoma 8B2-H6-10.7 deposited under accession number DSM ACC3249 with the depositary institute DSMZ on Sep. 12, 2014. It is shown herein that a monoclonal antibody was generated using a synthetic peptide with a c-terminal end sequence of the mutant calreticulin protein having the amino acid sequence RRKMSPARPRTSCREACLQGWTEA. It is therefore preferred that the herein provided antibodies specifically bind to RRKMSPARPRTSCREACLQGWTEA (or a fragment thereof or an epitope thereof).

The terms “recognizing”, “binding” and “detecting” as used in the context of the present invention are interchangeably used in the context of the present invention and define a binding (interaction) of at least two “antigen-interaction-sites” with each other. The term “antigen-interaction-site” defines, in accordance with the present invention, a motif of a polypeptide of the antibody which shows the capacity of specific interaction with a specific antigen or a specific group of antigens of the mutant calreticulin protein, in particular the C-terminus thereof (or a fragment) as defined herein. Said “recognition”, “binding” and “detection” is also understood to define a “specific recognition”.

Thus, the terms “recognizing”, “binding” and “detecting” as used in the context of the antibodies of the present invention and the method of generating such antibodies of the present invention refers in particular to a binding reaction that is determinative of the presence of mutant calreticulin, in particular the C-terminal part thereof, for example in the presence of a heterogeneous population of e.g. other biologics like wild-type calreticulin or other proteins.

Thus, under designated assay conditions, the specified antibodies and the mutant calreticulin, in particular the C-terminal part thereof, bind to one another and do not bind in a significant amount to other components present in a sample. A variety of immunoassay formats may be used to test antibodies specifically reactive with a particular antigen, i.e., mutant calreticulin, in particular the C-terminal part thereto. Such immunoassay formats and methods for identifying whether a specific immune reaction has been elicited are well-known to the person skilled in the art; see for example Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. Based on the teaching provided herein, the person skilled in the art is in a position to provide for and generate specific antibodies directed against the mutant calreticulin, in particular the C-terminal part thereof.

The term “recognizing”, “binding” and “detecting” as used in accordance with the present invention means in particular that the antibody of the invention does not or does not essentially cross-react wild-type calreticulin. Accordingly, the antibody of the invention specifically binds to/interacts with the mutant calreticulin, in particular the C-terminal part thereof (and fragment or epitopes thereof).

Cross-reactivity of the antibodies of the invention may be tested, for example, by assessing binding of said antibodies under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the mutant calreticulin, in particular the C-terminal part thereof, as well as to a number of more or less (structurally and/or functionally) closely related proteins. Only those antibodies that bind to the mutant calreticulin, in particular the C-terminal part thereof, but do not or do not essentially bind to any other related or unrelated protein are considered specific for the mutant calreticulin, in particular the C-terminal part thereof. Such antibodies can be used in accordance with the present invention. These methods may comprise, inter alia, binding studies, blocking and competition studies with structurally and/or functionally closely related molecules. These binding studies also comprise FACS analysis, surface plasmon resonance (SPR, e.g. with BIAcore®), analytical ultracentrifugation, isothermal titration calorimetry, fluorescence anisotropy, fluorescence spectroscopy or by radiolabeled ligand binding assays.

The term “specifically binding” means in accordance with this invention that the antibody/binding molecule is capable of specifically interacting with and/or binding to mutant calreticulin protein as defined herein. Therefore, said term relates to the specificity of the antibody, i.e. to its ability to discriminate between mutant calreticulin and another, non-mutant calreticulin protein. A “non-mutant calreticulin protein” may, for example, be a wild-type calreticulin protein. Generally a “non-mutant calreticulin protein” can be understood as a protein that does not present/comprise the unique C-terminal part of mutant calreticulin protein or a fragment/portion thereof. Specificity can be determined experimentally by methods known in the art. Such methods comprise, but are not limited to Western blots, ELISA-, RIA-, ECL-, IRMA-tests and peptide scans. Such methods also comprise the determination of KD-values.

As used herein, the term “antibody specifically binding to mutant calreticulin protein” therefore refers to an antibody or a functional fragment/derivative thereof that specifically binds to a mutant calreticulin protein (or a fragment or epitope of a mutant calreticulin protein) and that does not specifically bind to other non-mutant calreticulin proteins. Preferably, antibodies (or functional fragments thereof) binding specifically to a mutant calreticulin protein or fragment thereof do not non-specifically cross-react with other antigens (e.g., binding cannot be competed away with a non-mutant calreticulin polypeptide/protein, e.g., BSA in an appropriate immunoassay). Antibodies or functional fragments that specifically (or immunospecifically) bind to a polypeptide/protein can be identified, for example, by immunoassays or other techniques known to those of skill in the art.

In a certain aspect, antibodies provided or to be used in accordance with the present invention, bind to the same epitope(s) as any of the antibodies provided herein, wherein the latter antibodies comprise one or more of the CDR(s) and/or a V_{H}-region and/or a V_{L}-region and/or a heavy chain and/or a light chain as disclosed herein. For example, antibodies provided or to be used in accordance with the present invention, bind to the same epitope(s) as an antibody comprising a variable region of the heavy chain comprising a CDR-H3 region having an amino acid sequence as depicted in SEQ ID NO.: 3, or a CDR sequence having 75% or more amino acid identity to said CDR; or comprise the variable region of the heavy chain of said antibody comprising a CDR-H3 region having an amino acid sequence as depicted in SEQ ID NO.: 6, or a CDR sequence having 75% or more amino acid identity to said CDR.

The antibody, antibody fragment thereof or antibody derivatives of this invention bind selectively or specifically to an epitope of mutant calreticulin protein. The peptide scan (pepspot assay) is routinely employed to map linear epitopes in a polypeptide antigen. The primary sequence of the polypeptide is synthesized successively on activated cellulose with peptides overlapping one another. The recognition of certain peptides by the antibody to be tested for its ability to detect or recognize a specific antigen/epitope is scored by routine colour development (secondary antibody with horseradish peroxidase and 4-chloronaphthol and hydrogenperoxide), by a chemoluminescence reaction or similar means known in the art. In the case of, inter alia, chemoluminescence reactions, the reaction can be quantified. If the antibody reacts with a certain set of overlapping peptides one can deduce the minimum sequence of amino acids that are necessary for reaction. The same assay can reveal two distant clusters of reactive peptides, which indicate the recognition of a discontinuous, i. e. conformational epitope in the antigenic polypeptide (Geysen (1986), Mol. Immunol. 23, 709-715). In addition to the pepspot assay, standard ELISA assay can be carried out. Small hexapeptides may be coupled to a protein and coated to an immunoplate and reacted with antibodies to be tested. The scoring may be carried out by standard colour development (e.g. secondary antibody with horseradish peroxidase and tetramethyl benzidine with hydrogenperoxide). The reaction in certain wells is scored by the optical density, for example at 450 nm. Typical background (=negative reaction) may be 0.1 OD, typical positive reaction may be 1 OD. This means the difference (ratio) positive/negative can be more than 10 fold.

The antibody/antibodies of the present invention is directed against/binds specifically to mutant calreticulin protein, a fragment thereof or an epitope of mutant calreticulin protein, preferably to the C-terminal region of mutant calreticulin protein, for example, to the C-terminal region of mutant calreticulin protein as shown in SEQ ID NOs: 35 to 70. Preferably, the antibody/antibodies of the present invention bind specifically to mutant calreticulin protein that is present on the extracellular side of a plasma membrane. In other words, the antibody/antibodies of the present invention bind specifically to mutant calreticulin protein that is localized at the extracellular side of a plasma membrane. In one aspect, the antibody of this invention binds to or can be generated against a polypeptide having the full length C-terminal part of mutant calreticulin protein (or a fragment thereof).

Subject of the present invention are antibodies having the same or essentially the same biological activity as the herein defined by sequences of CDR(s)/variable regions and/or heavy and/or light chains or obtainable from hybridoma 8B2-H6-10.7 deposited under accession number DSM ACC3249 with the depositary institute DSMZ (Braunschweig, Germany) on Sep. 12, 2014.

The following relates to biological activities of the herein provided antibodies/antibodies to be used in accordance with the present invention.

It is envisaged herein that mutant calreticulin can be secreted. It is envisaged herein that mutant calreticulin can be present in the extracellular matrix. In certain aspects, the antibody/antibodies of the present invention bind(s) specifically to secreted mutant calreticulin protein. In certain aspects, the antibody/antibodies of the present invention bind(s) specifically to shedded mutant calreticulin protein. In certain aspects, the antibody/antibodies of the present invention bind(s) specifically to extracellular mutant calreticulin protein. In certain aspects, the antibody/antibodies of the present invention bind(s) specifically to mutant calreticulin protein that is present in the extracellular matrix. In one aspect, the antibody of this invention binds to or can be generated against a polypeptide having the full length C-terminal part of mutant calreticulin protein. The antibody/antibodies of the present invention can be directed against/bind(s) specifically to mutant calreticulin protein, a fragment thereof or an epitope of mutant calreticulin protein, preferably to the C-terminal region of mutant calreticulin, for example, to the C-terminal region of mutant calreticulin as shown in SEQ ID NOs: 35 to 70. The sample can, for example, be a blood samples, a serum sample or a bone marrow sample. Any technique for protein detection can be used including but not limited to immunologic methodologies, such as immunostaining (e.g. of patient material/histological samples), immunohistochemistry (IHC), immunocytochemistry, Western blot, ELISA immunoassay, gel- or blot-based methods, mass spectrometry, flow cytometry, or fluorescent activated cell sorting (FACS). FACS analysis can also be performed on cells fixed in formaldehyde/paraformaldehyde.

For example, anti-mutant CALR protein polyclonal antibody (e.g. polyclonal antibody from Rabbit) can be used in immunologic methods (e.g. for immunostaining). An exemplary antibody to be used in such immunologic methods is disclosed in Vannucchi (Leukemia. 2014 September; 28(9):1811-8. doi: 10.1038/leu.2014.100. Epub 2014 Mar. 12).

In a certain aspect, the present invention relates to a method for diagnosing a myeloid malignancy, comprising detecting or assaying a mutant calreticulin protein in a biological sample of an individual suspected of suffering from a myeloid malignancy or suspected of being prone to suffering from a myeloid malignancy using the herein provided antibody or an antibody specifically binding to mutant calreticulin protein. The herein provided methods for diagnosing are preferably in vitro methods. Preferably, the antibody specifically binds to the C-terminal part of mutant calreticulin protein or to a part of the C-terminal part of mutant calreticulin protein. Exemplary C-terminal parts of mutant calreticulin protein is shown in any one of SEQ ID NOs: 35 to 70. An exemplary part of the C-terminal part of mutant calreticulin protein is shown in SEQ ID NO: 71. The biological sample can be a blood sample, a bone marrow sample or a serum sample. Mutant calreticulin protein can be detected or assayed by any protein detection methods, including but not limited to immunologic methodologies/techniques, such as immunohistochemistry (IHC), immunocytochemistry, Western blot, or ELISA immunoassay; gel- or blot-based methods; mass spectrometry; flow cytometry; or fluorescent activated cell sorting (FACS). FACS analysis can also be performed on cells fixed in formaldehyde/paraformaldehyde. Immunologic methodologies/techniques, such as immunohistochemistry (IHC), immunocytochemistry, Western blot, or ELISA immunoassay, are preferred in the context of detection/assaying secreted or shedded mutant calreticulin in a sample, e.g. in a serum sample.

The antibody provided herein can have the capacity to specifically bind/recognize mutant calreticulin protein (or an epitope thereof) when the protein is present on the surface of a cell or when the protein is present on the extracellular side of a plasma membrane or when the protein is localized at the extracellular side of a plasma membrane. The cells can express mutant calreticulin protein. The cells can be part of a sample from a patient. The cells can be derived from (e.g. purified from) a sample from a patient). The cells can be intact, living or whole cells or fixed in formaldehyde/paraformaldehyde. The sample can, for example, be a blood samples, a serum sample or a bone marrow sample.

The antibody provided herein can have the capacity to specifically bind/recognize mutant calreticulin protein (or an epitope thereof) when the protein is present on the surface of a cell expressing mutant calreticulin protein or when the protein is present on the extracellular side of a plasma membrane of a cell expressing mutant calreticulin protein or when the protein is localized at the extracellular side of a plasma membrane of a cell expressing mutant calreticulin protein.

In a certain aspect, the present invention relates to a method for diagnosing a myeloid malignancy, comprising detecting or assaying a mutant calreticulin protein in a biological sample of an individual suspected of suffering from a myeloid malignancy or suspected of being prone to suffering from a myeloid malignancy using the antibody of the herein provided or an antibody specifically binding to mutant calreticulin protein. Preferably, the antibody specifically binds to the C-terminal part of mutant calreticulin protein or to a part of the C-terminal part of mutant calreticulin protein. Exemplary C-terminal parts of mutant calreticulin protein is shown in any one of SEQ ID NOs: 35 to 70. An exemplary part of the C-terminal part of mutant calreticulin protein is shown in SEQ ID NO: 71. In a preferred aspect, the mutant calreticulin protein is present on the extracellular side of a plasma membrane of a cell. In a preferred aspect, the mutant calreticulin protein is present on surface of a cell. In a preferred aspect, the mutant calreticulin protein is localized at the extracellular side of a plasma membrane. The cell is preferably a living cell, whole cell or intact cell. In this context, the detection or the assay of mutant calreticulin protein is preferably performed using a flow cytometry technique. Particularly preferred is fluorescent activated cell sorting (FACS). In this context, it is preferred that the biological sample is a blood sample or a bone marrow sample. FACS analysis can also be performed on cells fixed in formaldehyde/paraformaldehyde.

Generally, the antibodies provided and to be used in accordance with the present invention may comprise a CDR sequence having 75% or more (e.g. 80%, more preferably 85%, 90%, most preferably 95%, 96%, 97%, 98%, 99% or more) amino acid identity to one of the specific CDR sequences provided and disclosed herein. It is understood that the identity is assessed/determined over the full length of the CDR sequence.

The term “CDR” as employed herein relates to “complementary determining region”, which is well known in the art. The CDRs are parts of immunoglobulins and T cell receptors that determine the specificity of said molecules and make contact with specific ligand. The CDRs are the most variable part of the molecule and contribute to the diversity of these molecules. There are three CDR regions, CDR1, CDR2 and CDR3, in each V domain. CDR-H depicts a CDR region of a variable heavy chain and CDR-L relates to a CDR region of a variable light chain. H means the variable heavy chain and L means the variable light chain. The CDR regions of an Ig-derived region may be determined as described in Kabat (1991), Sequences of Proteins of Immunological Interest, 5th edit., NIH Publication no. 91-3242 U.S. Department of Health and Human Services; Chothia (1987), J. Mol. Biol. 196, 901-917; and Chothia (1989) Nature, 342, 877-883.

Each CDR region of a variable heavy chain is herein interchangeably designated as CDR-H1 or VH-CDR1, CDR-H2 or VH-CDR2, and CDR-H3 or VH-CDR3, respectively. Likewise, each CDR region of a variable light chain is designated herein CDR-L1 or VL-CDR1, CDR-L2 or VL-CDR2, and CDR-L3 or VL-CDR3, respectively.

In one aspect, the variable region of the heavy chain of the antibody of this invention comprises a CDR-H3 region having an amino acid sequence as depicted in SEQ ID NO.: 3 or SEQ ID NO.: 6. The antibodies may also comprise a CDR sequence having 75% or more (e.g. 80%, more preferably 85%, 90%, most preferably 95%, 96%, 97%, 98%, 99% or more) amino acid identity to one of said CDRs.

In a certain aspect the present invention relates to an antibody that specifically binds to a mutant calreticulin protein,

wherein the variable region of the heavy chain of said antibody comprises a CDR-H3 region having an amino acid sequence as depicted in SEQ ID NO.: 3, or a CDR sequence having 75% or more amino acid identity to said CDR;
or
wherein the variable region of the heavy chain of said antibody comprises a CDR-H3 region having an amino acid sequence as depicted in SEQ ID NO.: 6, or a CDR sequence having 75% or more amino acid identity to said CDR.

The antibody of the present invention can comprise

A variable region of the heavy chain comprising a CDR-H1 region having an amino acid sequence as depicted in SEQ ID NO: 1, or a CDR sequence having 75% or more amino acid identity to said CDR;
or
a variable region of the heavy chain comprising a CDR-H1 region having an amino acid sequence as depicted in SEQ ID NO: 4, or a CDR sequence having 75% or more amino acid identity to said CDR.

The antibody of the present invention can comprise

a variable region of the heavy chain comprising a CDR-H2 region having an amino acid sequence as depicted in SEQ ID NO: 2, or a CDR sequence having 75% or more amino acid identity to said CDR;
or
a variable region of the heavy chain comprising a CDR-H2 region having an amino acid sequence as depicted in SEQ ID NO: 5, or a CDR sequence having 75% or more amino acid identity to said CDR.

In a certain aspect the present invention relates to an antibody that specifically binds to a mutant calreticulin protein,

wherein the variable region of the heavy chain of said antibody comprises a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3, or a CDR sequence having 75% or more amino acid identity to one of said CDRs.

The antibody of the present invention can comprise a variable region of the heavy chain comprising a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3.

In a certain aspect the present invention relates to an antibody that specifically binds to a mutant calreticulin protein,

wherein the variable region of the heavy chain of said antibody comprises a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6, or a CDR sequence having 75% or more amino acid identity to one of said CDRs.

The antibody of the present invention can comprise a variable region of the heavy chain comprising a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6.

The antibody of the present invention can comprise a variable region of the light chain comprising a CDR-L1 region having an amino acid sequence as depicted in SEQ ID NO: 7, or a CDR sequence having 75% or more amino acid identity to said CDR.

The antibody of the present invention can comprise a variable region of the light chain comprising a CDR-L2 region having an amino acid sequence as depicted in SEQ ID NO: 8, or a CDR sequence having 75% or more amino acid identity to said CDR.

The antibody of the present invention can comprise a variable region of the light chain comprising a CDR-L3 region having an amino acid sequence as depicted in SEQ ID NO: 9, or a CDR sequence having 75% or more amino acid identity to said CDR.

The antibody of the present invention can comprise a variable region of the light chain comprising a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

In a certain aspect the present invention relates to an antibody that specifically binds to a mutant calreticulin protein,

wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

The antibody of the present invention can comprise a variable region of the light chain comprising a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

In a certain aspect the present invention relates to an antibody that specifically binds to a mutant calreticulin protein,

wherein the variable region of the heavy chain of said antibody comprises a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3, or a CDR sequence having 75% or more amino acid identity to one of said CDRs;
and
wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.
The antibody of the present invention can comprise
a variable region of the heavy chain comprising a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3;
and
a variable region of the light chain comprising a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

In a certain aspect the present invention relates to an antibody that specifically binds to a mutant calreticulin protein,

wherein the variable region of the heavy chain of said antibody comprises a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6, or a CDR sequence having 75% or more amino acid identity to one of said CDRs;
and
wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

The antibody of the present invention can comprise

a variable region of the heavy chain comprising a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6;
and

a variable region of the light chain comprising a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

The herein provided antibodies can comprise one or more of the heavy or light chain variable sequences above or a sequence at least 75%, 80%, more preferably at least 85%, 90%, even more preferably at least 95%, 96%, 97%, 98%, or most preferably 99% identical thereto.

In one aspect, the variation in the sequences occurs in the framework regions, i.e. outside of the CDR sequences. For example, the antibodies of these aspects contain specific CDR regions above that are not subject to variation. Yet, the framework region of these antibodies can show a variation/identity of 75% or more (or 80%, more preferably at least 85%, 90%, even more preferably at least 95%, 96%, 97%, 98%, or most preferably 99%) to the framework region of the specific variable V_{L}-region(s) and/or variable V_{H}-region(s) as defined above. The framework region(s) can be identified by methods known in the art. As used herein the term “framework region” can refer to the sequence of the variable V_{L}-region(s) and/or the variable V_{H}-region(s) that is outside of the CDR sequences.

In a certain aspect the present invention relates to an antibody that specifically binds to a mutant calreticulin protein,

wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region;
and/or
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region,
said antibody comprising
a variable region of the heavy chain comprising a CDR-H1 region having an amino acid sequence as depicted in SEQ ID NO: 1, a CDR-H2 region having an amino acid sequence as depicted in SEQ ID NO: 2, and/or a CDR-H3 region having an amino acid sequence as depicted in SEQ ID NO.: 3;
and/or
a variable region of the light chain comprising a CDR-L1 region having an amino acid sequence as depicted in SEQ ID NO: 7, a CDR-L2 region having an amino acid sequence as depicted in SEQ ID NO: 8, and/or a CDR-L3 region having an amino acid sequence as depicted in SEQ ID NO: 9.

In a certain aspect the present invention relates to an antibody that specifically binds to a mutant calreticulin protein,

wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region;
and/or
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region; or
a variable V_{L}-region having an amino acid sequence as shown in SEQ ID NO:15, or a variable V_{L}-region having an amino acid sequence which has 75% or more identity to said variable V_{L}-region,
said antibody comprising
a variable region of the heavy chain comprising a CDR-H1 region having an amino acid sequence as depicted in SEQ ID NO: 4, a CDR-H2 region having an amino acid sequence as depicted in SEQ ID NO: 5, and/or a CDR-H3 region having an amino acid sequence as depicted in SEQ ID NO.: 6;
and/or
a variable region of the light chain comprising a CDR-L1 region having an amino acid sequence as depicted in SEQ ID NO: 7, a CDR-L2 region having an amino acid sequence as depicted in SEQ ID NO: 8, and/or a CDR-L3 region having an amino acid sequence as depicted in SEQ ID NO: 9.

In a certain aspect the present invention relates to an antibody that specifically binds to a mutant calreticulin protein,

wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region;
and
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region,
said antibody comprising
a variable region of the heavy chain comprising a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3;
and
a variable region of the light chain comprising a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

In a certain aspect the present invention relates to an antibody that specifically binds to a mutant calreticulin protein,

wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region;
and
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region; or
a variable V_{L}-region having an amino acid sequence as shown in SEQ ID NO:15, or a variable V_{L}-region having an amino acid sequence which has 75% or more identity to said variable V_{L}-region,
said antibody comprising
a variable region of the heavy chain comprising a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6;
and
a variable region of the light chain comprising a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

The antibody of the present invention can comprise

a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region.

In a certain aspect the present invention relates to an antibody that specifically binds to a mutant calreticulin protein,

wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region.

The antibody of the present invention can comprise

a variable V_{H}-region as encoded by a nucleic acid molecule as shown in SEQ ID NO:10; or
a variable V_{H}-region having an amino acid sequence as shown in SEQ ID NO:11.

The antibody of the present invention can comprise

a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region.

In a certain aspect the present invention relates to an antibody that specifically binds to a mutant calreticulin protein,

wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region.

The antibody of the present invention can comprise

a variable V_{H}-region as encoded by a nucleic acid molecule as shown in SEQ ID NO:12; or
a variable V_{H}-region having an amino acid sequence as shown in SEQ ID NO:13.

The antibody of the present invention can comprise

a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region.

In a certain aspect the present invention relates to an antibody that specifically binds to a mutant calreticulin protein,

wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region.

The antibody of the present invention can comprise

a variable V_{L}-region as encoded by a nucleic acid molecule as shown in SEQ ID NO:14, or
a variable V_{L}-region having an amino acid sequence as shown in SEQ ID NO:15.

In a certain aspect the present invention relates to an antibody that specifically binds to a mutant calreticulin protein,

wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region;
and
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region.

The antibody of the present invention can comprise

a variable V_{H}-region as encoded by a nucleic acid molecule as shown in SEQ ID NO:10; or
a variable V_{H}-region having an amino acid sequence as shown in SEQ ID NO:11;
and
wherein said antibody comprises a variable V_{L}-region as encoded by a nucleic acid molecule as shown in SEQ ID NO:14, or
a variable V_{L}-region having an amino acid sequence as shown in SEQ ID NO:15.

In a certain aspect the present invention relates to an antibody that specifically binds to a mutant calreticulin protein,

wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region;
and
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region; or
a variable V_{L}-region having an amino acid sequence as shown in SEQ ID NO:15, or a variable V_{L}-region having an amino acid sequence which has 75% or more identity to said variable V_{L}-region.

The antibody of the present invention can comprise

a variable V_{H}-region as encoded by a nucleic acid molecule as shown in SEQ ID NO:12; or
a variable V_{H}-region having an amino acid sequence as shown in SEQ ID NO:13; a
and
a variable V_{L}-region as encoded by a nucleic acid molecule as shown in SEQ ID NO:14, or
a variable V_{L}-region having an amino acid sequence as shown in SEQ ID NO:15.

The antibody of the present invention can comprise

a heavy chain as encoded by a nucleic acid molecule as shown in SEQ ID NO:16, or a heavy chain as encoded by a nucleic acid molecule having 75% or more identity to said heavy chain; or
a heavy chain having an amino acid sequence as shown in SEQ ID NO:17, or a heavy chain having an amino acid sequence which has 75% or more identity to said heavy chain.

In a certain aspect the present invention relates to an antibody that specifically binds to a mutant calreticulin protein,

wherein said antibody comprises a heavy chain as encoded by a nucleic acid molecule as shown in SEQ ID NO:16, or a heavy chain as encoded by a nucleic acid molecule having 75% or more identity to said heavy chain; or
a heavy chain having an amino acid sequence as shown in SEQ ID NO:17, or a heavy chain having an amino acid sequence which has 75% or more identity to said heavy chain.

The antibody of the present invention can comprise

a heavy chain as encoded by a nucleic acid molecule as shown in SEQ ID NO:16; or
a heavy chain having an amino acid sequence as shown in SEQ ID NO:17.

The antibody of the present invention can comprise

a heavy chain as encoded by a nucleic acid molecule as shown in SEQ ID NO:18, or a heavy chain as encoded by a nucleic acid molecule having 75% or more identity to said heavy chain;
or
a heavy chain having an amino acid sequence as shown in SEQ ID NO:19, or a heavy chain having an amino acid sequence which has 75% or more identity to said heavy chain.

In a certain aspect the present invention relates to an antibody that specifically binds to a mutant calreticulin protein,

wherein said antibody comprises a heavy chain as encoded by a nucleic acid molecule as shown in SEQ ID NO:18, or a heavy chain as encoded by a nucleic acid molecule having 75% or more identity to said heavy chain; or
a heavy chain having an amino acid sequence as shown in SEQ ID NO:19, or a heavy chain having an amino acid sequence which has 75% or more identity to said heavy chain.

The antibody of the present invention can comprise

a heavy chain as encoded by a nucleic acid molecule as shown in SEQ ID NO:18; or
a heavy chain having an amino acid sequence as shown in SEQ ID NO:19.

The antibody of the present invention can comprise a

a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

In a certain aspect the present invention relates to an antibody that specifically binds to a mutant calreticulin protein,

wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

The antibody of the present invention can comprise

a light chain as encoded by a nucleic acid molecule as shown in SEQ ID NO:20; or
a variable V_{H}-region having an amino acid sequence as shown in SEQ ID NO:21.

In a certain aspect the present invention relates to an antibody that specifically binds to a mutant calreticulin protein,

wherein said antibody comprises a heavy chain as encoded by a nucleic acid molecule as shown in SEQ ID NO:16, or a heavy chain as encoded by a nucleic acid molecule having 75% or more identity to said heavy chain; or
a heavy chain having an amino acid sequence as shown in SEQ ID NO:17, or a heavy chain having an amino acid sequence which has 75% or more identity to said heavy chain;
and
wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

The antibody of the present invention can comprise

a heavy chain as encoded by a nucleic acid molecule as shown in SEQ ID NO:16; or
a heavy chain having an amino acid sequence as shown in SEQ ID NO:17;
and
wherein said antibody comprises a light chain as encoded by a nucleic acid molecule as shown in SEQ ID NO:20; or
a variable V_{H}-region having an amino acid sequence as shown in SEQ ID NO:21.

In a certain aspect the present invention relates to an antibody that specifically binds to a mutant calreticulin protein,

wherein said antibody comprises a heavy chain as encoded by a nucleic acid molecule as shown in SEQ ID NO:18, or a heavy chain as encoded by a nucleic acid molecule having 75% or more identity to said heavy chain; or
a heavy chain having an amino acid sequence as shown in SEQ ID NO:19, or a heavy chain having an amino acid sequence which has 75% or more identity to said heavy chain;
and
wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

The antibody of the present invention can comprise

a heavy chain as encoded by a nucleic acid molecule as shown in SEQ ID NO:18; or
a heavy chain having an amino acid sequence as shown in SEQ ID NO:19;
and
wherein said antibody comprises a light chain as encoded by a nucleic acid molecule as shown in SEQ ID NO:20; or
a variable V_{H}-region having an amino acid sequence as shown in SEQ ID NO:21.

The antibodies/binding molecules of the invention include the antibodies having one or more of the CDRs and/or one or more of the variable regions (V_{H}-region and/or V_{L}-region) and/or one or more of the chains (heavy chain and/or light chain) as disclosed herein as well as variants thereof having 75% or more (for example 80%, more preferably 85%, 90%, most preferably 95%, 96%, 97%, 98%, or 99%) sequence identity to said CDR(s), variable region(s) and/or chains.

As used herein, the terms “identity”, “sequence identity”, “homology” or “sequence homology” (the terms are used interchangeably herein) are used to describe the sequence relationships between two or more amino acid sequences, proteins (or fragments thereof), or polypeptides (or fragments thereof), or corresponding nucleic acid sequences, nucleic acids (or fragments thereof), polynucleotides (or fragments thereof). The terms can be understood in the context of and in conjunction with the terms including: (a) reference sequence, (b) comparison window, (c) sequence identity, (d) percentage of sequence identity, and (e) substantial identity or “homologous”.

A “reference sequence” is a defined sequence used as a basis for sequence comparison. A reference sequence may be a subset of or the entirety of a specified sequence.

A “comparison window” includes reference to a contiguous and specified segment of a nucleic acid sequence/polynucleotide sequence or amino acid sequence/polypeptide sequence/protein sequence, wherein the nucleic acid sequence/polynucleotide sequence or amino acid sequence/polypeptide sequence/protein sequence may be compared to a reference sequence. The portion of the nucleic acid sequence/polynucleotide sequence or amino acid sequence/polypeptide sequence/protein sequence in the comparison window may comprise additions, substitutions, or deletions (i.e., gaps) compared to the reference sequence (which does not comprise additions, substitutions, or deletions) for optimal alignment of the two sequences. Generally, the comparison window may be at least about 9 contiguous nucleotides in length (or correspondingly about 3 amino acid residues in length), and optionally can be about 9, 12, 15, 18, 21, 24, 27, 30, 33, 36, 39, 40, 50, or 100, contiguous nucleotides or longer (or correspondingly about 3, 4, 5, 6, 7, 8, 9, 11, 13, 16, or 33 amino acid residues in length or longer). Those of skill in the art understand that to avoid a misleadingly high similarity to a reference sequence due to inclusion of gaps in the polynucleotide or polypeptide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp (1988) Gene 73: 237-244; Corpet et al. (1988) Nucleic Acids Research 16:881-90; Huang, et al. (1992) Computer Applications in the Biosciences, 8:1-6; and Pearson, et al. (1994) Methods in Molecular Biology, 24:7-331. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters. Altschul et al. (1997) Nucleic Acids Res, 2:3389-3402. It is to be understood that default settings of these parameters can be readily changed as needed in the future.

As those ordinary skilled in the art will understand, BLAST searches assume that proteins or nucleic acids can be modeled as random sequences. However, many real proteins and nucleic acids comprise regions of nonrandom sequences which may be homopolymeric tracts, short-period repeats, or regions enriched in one or more amino acids or nucleic acids. Such low-complexity regions may be aligned between unrelated proteins even though other regions of the protein or nucleic acid are entirely dissimilar. A number of low-complexity filter programs can be employed to reduce such low-complexity alignments. For example, the SEG (Wooten et al. (1993) Comput. Chem. 17:149-163) and XNU (Claverie et al. (1993) Comput. Chem. 17:191-1) low-complexity filters can be employed alone or in combination.

“Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Percentage of sequence identity” means the value determined by comparing two optimally aligned sequences over a comparison window, wherein the portion of the polynucleotide or nucleic acid sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 75% sequence identity, preferably at least 80%, more preferably at least 85%, still more preferably at least 90% and even more preferably at least 95%, 96%, 97%, 98% or 99%, compared to a reference sequence using one of the alignment programs described using standard parameters. These values can be appropriately adjusted to determine corresponding identity of proteins encoded by two nucleotide sequences by taking into account codon degeneracy, amino acid similarity, reading frame positioning and the like. Accordingly, the present invention provides for binding molecules/antibodies etc specifically binding to a mutant calreticulin protein which comprise CDRs and/or variable regions and/or heavy/light chains that are encoded by nucleic acid sequences/molecules that have at least 75% sequence identity, more preferably at least 80%, even more preferably at least 85%, still more preferably at least 90% and most preferably at least 95%, 96%, 97%, 98% or 99% sequence identity with the corresponding nucleic acid sequences/molecules encoding the amino acid sequence of an antibody (or variable regions thereof or CDRs thereof or heavy/light chains thereof, respectively) that can be obtained or is obtainable from hybridoma 8B2-H6-10.7 deposited under accession number DSM ACC3249 with the depositary institute DSMZ (Braunschweig, Germany) on Sep. 12, 2014.

Another indication that nucleotide/nucleic acid sequences are substantially identical is if two molecules hybridize to each other under stringent conditions. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 55° C. or 60° C. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

The term “substantial identity” or “homologous” in their various grammatical forms in the context of peptides indicates that a peptide comprises a sequence that has a desired identity, for example, at least 75% sequence identity to a reference sequence, preferably at least 80% sequence identity to a reference sequence, more preferably 85%, even more preferably at least 90% or 95% or even 96%, 97%, 98% or 99% sequence identity to the reference sequence over a specified comparison window. Preferably, optimal alignment is conducted using the homology alignment algorithm of Needleman and Wunsch (1970) J. Mol. Biol., 48:443. An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes. Accordingly, the present invention provides for binding molecules/antibodies etc specifically binding to a mutant calreticulin protein which comprise CDRs and/or variable regions and/or heavy/light chains that have an amino acid sequence having at least 75% sequence identity, more preferably at least 80%, even more preferably at least 85%, still more preferably at least 90% and most preferably at least 95%, 96%, 97%, 98% or 99% sequence identity with the amino acid sequence of an antibody (or variable regions thereof or CDRs thereof or heavy/light chains thereof, respectively) that can be obtained or is obtainable from hybridoma 8B2-H6-10.7 deposited under accession number DSM ACC3249 with the depositary institute DSMZ (Braunschweig, Germany) on Sep. 12, 2014.

Conservative amino acid substitutions are known to those skilled in the art and typically include, but are not limited to, substitutions listed in the following table:

| | | | Exemplary | Typical |
|---|---|---|---|---|
| | | | Conservative | substi- |
| α-Aminoacid | Symbol | Nature | substitutions | tution |
| Alanine | Ala (A) | Aliphatic, hydro- | Val, Ile, Leu, | Val |
| | | phobic, neutral | Gly, Ser | |
| Arginine | Arg (R) | Polar, hydrophil- | Lys, His, Gln, | Lys |
| | | ic, charge (+) | Asn | |
| Asparagine | Asn (N) | Polar, hydrophil- | Lys, His, Gln, | Gln |
| | | ic, neutral | Arg | |
| Cysteine | Cys (C) | Polar, hydro- | Ser, Ala | Ser |
| | | phobic, neutral | | |
| Glutamine | Gln (Q) | Polar, hydro- | Asn | Asn |
| | | phobic, neutral | | |
| Glycine | Gly (G) | Aliphatic, | Pro, Ala | Ala |
| | | neutral | | |
| Histidine | His (H) | Aromatic, polar, | Asn, Gln, Lys, | Arg |
| | | hydrophilic, | Arg | |
| | | charge (+) | | |
| Isoleucine | Ile (I) | Aliphatic, hydro- | Leu, Val, Met, | Leu |
| | | phobic, neutral | Ala, Phe | |
| Leucine | Leu (L) | Aliphatic, hydro- | Ile, Val, Met, | Ile |
| | | phobic, neutral | Phe, Ala | |
| Lysine | Lys (K) | polar, hydrophil- | Arg, Gln, Asn, | Arg |
| | | ic, charge (+) | His | |
| Methionine | Met (M) | hydrophobic, | Leu, Ile, Phe | Leu |
| | | neutral | | |
| Phenylalanine | Phe (F) | Aromatic, hydro- | Leu, Ile, Val, | Leu |
| | | phobic, neutral | Ala, Tyr | |
| Proline | Pro (P) | hydrophobic, | Ala, Gly | Gly |
| | | neutral | | |
| Serine | Ser (S) | Polar, hydrophil- | Thr, Ala, Cys | Thr |
| | | ic, neutral | | |
| Threonine | Thr (T) | Polar, hydrophil- | Ser | Ser |
| | | ic, neutral | | |
| Tryptophan | Trp (W) | Aromatic, hydro- | Tyr, Phe | Tyr |
| | | phobic, neutral | | |
| Tyrosine | Tyr (Y) | Aromatic, polar, | Trp, Phe, Thr, | Phe |
| | | hydrophobic | Ser | |
| Valine | Val (V) | Aliphatic, hydro- | Ile, Met, Leu, | Leu |
| | | phobic, neutral | Phe, Ala, | |
| Glutamic Acid | Glu (E) | Polar, hydrophil- | Asp, Gln | Asp |
| | | ic, charge (−) | | |
| Aspartic Acid | Asp (D) | Polar, hydrophil- | Glu, Asn | Glu |
| | | ic, charge (−) | | |

In a certain aspect, the invention relates to antibodies/binding molecules that that specifically bind to a mutant calreticulin protein wherein said antibodies or binding molecules comprise one or more CDR sequences and/or a variable V_{H}-region and/or a variable V_{L}-region variable regions and/or heavy/light chains as disclosed herein, with the exception that the one or more CDR sequences and/or variable V_{H}-region and/or variable V_{L}-region and/or heavy chain and/or light chain have 1 or more amino acid substitutions, deletions or additions. For example, the antibodies/binding molecules that specifically bind to a mutant calreticulin protein can comprise one or more CDR(s) as disclosed herein with the exception that the CDR(s) have 1 or more, preferably 1, 2 or 3, more preferably 1 or 2 amino acid substitutions, deletions or additions. For example, the antibodies/binding molecules that specifically bind to a mutant calreticulin protein can comprise variable regions as disclosed herein with the exception that the regions have up to 20, preferably up to 15, more preferably up to 10, amino acid substitutions, deletions or additions. For example, the antibodies/binding molecules that specifically bind to a mutant calreticulin protein can comprise a heavy and/or a light chain as disclosed herein with the exception that the heavy and/or a light chain have up to 20, preferably up to 15, more preferably up to 10, amino acid substitutions, deletions or additions.

In the context herein above, and in particular in relation to CDR sequences, amino acid substitutions are preferred. For example, the antibodies/binding molecules that specifically bind to a mutant calreticulin protein can comprise one or more CDR(s) as disclosed herein with the exception that the CDR(s) have 1 or more, preferably 1, 2 or 3, more preferably 1 or 2 amino acid substitutions, preferably conservative amino acid substitutions. For example, the antibodies/binding molecules that specifically bind to a mutant calreticulin protein can comprise variable regions as disclosed herein with the exception that the regions have up to 20, preferably up to 15, more preferably up to 10, amino acid substitutions, preferably conservative amino acid substitutions. For example, the antibodies/binding molecules that specifically bind to a mutant calreticulin protein can comprise a heavy and/or a light chain as disclosed herein with the exception that the heavy and/or a light chain have up to 20, preferably up to 15, more preferably up to 10, amino acid substitutions, preferably conservative amino acid substitutions.

The present invention provides antibodies comprising CDRs and/or variable sequences as described herein, or variants thereof, as disclosed above. Methods are known to those skilled in the art to modify the sequence of an existing antibody (parent antibody) to derive variant antibodies with high sequence homology to the sequence of the existing antibody that retain the capacity to specifically bind to the original target (a mutant calreticulin protein or, in particular, an epitope thereof).

Variant antibodies specifically binding to a mutant calreticulin protein (with similar or improved affinity, with modified selectivity, antigenicity, with modified pharmacokinetic characteristics) can be readily derived from the antibodies specifically binding to a mutant calreticulin protein disclosed herein through variation of the sequence of the disclosed or deposited antibodies, using methods that have been described in the literature.

Mutations can be introduced randomly into the variable regions of antibody genes by error-prone polymerase chain reaction (PCR) or E. coli mutator strains, site-directed mutagenesis, saturation mutagenesis, parsimonious mutagenesis, CDR walking or look-through mutagenesis targeting certain regions like the CDRs, hence generating limited collections of the specific variants of the parent antibody. Shuffling approaches include DNA shuffling, chain shuffling, or CDR shuffling to obtain shuffled variants of the parent antibody.

Introduction of variations can be random (radiation, chemical mutagens, error prone PCR, chain shuffling) or directed (site directed mutagenesis, (partial) gene synthesis using regular phosphoramidite chemistry or triplet synthesis). Random mutation efforts can be combined with in vitro selection procedures (i.e. display methods) to identify binders.

Directed mutagenesis is preferentially performed after in silico modeling of the mutant calreticulin protein−antibody specifically binding to the mutant calreticulin protein using the sequence and structure information of the (extracellular part of) the mutant calreticulin protein and the antibody specifically binding thereto.

Modeling can be done using the experimentally determined 3D crystal structure of the complex formed between the (extracellular domain of) mutant calreticulin protein with the antibodies specifically binding thereto of the invention as a starting point. Alternatively, modeling can also be done by using an in silico docking model of the (extracellular domain of) a mutant calreticulin protein and the antibodies disclosed herein based on published 3D structures of the individual protein.

The 3D structure of the antibody specifically binding to a mutant calreticulin protein can be predicted with one of different algorithms available in the art that are rapidly increasing in accuracy like: Web Antibody Modeling (WAM) (Whitelegg and Rees, Protein Eng. 2000; 14(12):819-824), Prediction of ImmunGlobulin Structure (PIGS) (Marcatili et al., Bioinformatics. 2008; 14(17):1953-1954), or RosettaAntibody (Sivasubramanian et al., Proteins. 2009; 14(2): 497-514.),).

The algorithms cited above can be used to dock the antibodies to the (extracellular domain of the) target protein; and to analyze sequence tolerance to variation with respect to the antibody-target protein binding capacity, i.e. the algorithms can be used by a skilled user to design variant antibodies binding the same epitope (see e.g. Barderas et al. Proc Natl Acad Sci USA. Jul. 1, 2008; 105(26): 9029-9034) and this principle can be applied to the mutant calreticulin protein (extracellular domain) binding antibodies with one or more of the CDRs and/or variable regions and/or the heavy chain and/or light chain as disclosed herein.

Typically, variations in a limited number of amino acids will be evaluated during in silico modeling. The effects of the variation may vary the affinity of the antibody to the mutant calreticulin protein target epitope: Typically it will be desirable that the affinity is similar or higher than that of the mutant calreticulin protein binding antibodies as disclosed herein. Focused libraries containing candidate daughter sequences with the desired variations can then be synthesized or produced by directed mutagenesis into the mutant calreticulin protein antibody sequences disclosed and provided herein. The retention of the a mutant calreticulin protein binding capacity can be verified after expressing the derived protein(s), and competition experiments can be used to demonstrate that the variant a mutant calreticulin protein antibodies derived from the antibodies as disclosed herein specifically bind to the mutant calreticulin protein (or the same original epitope thereof).

This process can be reiterated by submitting successful daughter sequence(s) to a new cycle of modifications, or to introduce stabilizing peripheral mutations. It has been described that the introduction of amino acid changes that increase affinity may reduce overall antibody protein stability, and that this may also lead to reduced expression/production of antibody (fragments) in mammalian cells (Wang et al., Proc Natl Acad Sci USA. Mar. 12, 2013; 110(11): 4261-4266). Stabilizing mutations can be identified by assessing melting curves using thermal scanning or light scattering [aggregation (agg)] of antibodies. Stabilizing mutations have been shown to stabilize antibodies independently of their target binding capacities. Mutations stabilizing the antibodies of the invention can be identified either directly starting from these antibodies, or using antibodies derived from the antibodies disclosed herein that have lost the mutant calreticulin protein binding capacity and then introduced into the antibodies of the invention or from the antibodies with mutant calreticulin protein binding capacity derived from them as described above.

Additional changes may be introduced into the antibodies of the invention to modify potential antigenicity, glycosylation, and antibodies may also be produced in different hosts to modify glycosylation. Said antibodies can contain the mutant calreticulin protein binding region from the antibody sequences as disclosed herein or they will be directly derived from them following established methods as disclosed above and will thus retain the binding capacity to the original epitope, as described above.

In all the aspects described herein, the antibody/binding molecule of the present invention may be a full antibody (immunoglobulin), an antibody fragment such as a F(ab)-fragment, a F(ab)2-fragment or an epitope-binding fragment, as well as a single-chain antibody. The antibodies/binding molecules of the invention may be a monoclonal antibody, a recombinantly produced antibody, a chimeric antibody, a humanized antibody, a human antibody, a fully human antibody, a CDR-grafted antibody, a bivalent antibody-construct, a synthetic antibody or a cross-cloned antibody, a diabody, a triabody, a tetrabody, a single chain antibody, a bispecific single chain antibody, etc. The antibody may also be a multispecific antibody, including a bi-specific antibody. The antibodies of the invention may be multifunctional, i.e. they may exert their effects via more than one mode of action, such as for example by activating ADCC or CDC pathways.

Thus, the antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above.

“Single-chain Fv” or “scFv” antibody fragments have, in the context of the invention, the VH and VL domains of an antibody, wherein these domains are present in a single polypeptide chain. Generally, the scFv polypeptide further comprises a polypeptide linker between the VH and VL domains which enables the scFv to form the desired structure for antigen binding. Techniques described for the production of single chain antibodies are described, e.g., in Plûckthun in The Pharmacology of Monoclonal Antibodies, Rosenburg and Moore eds. Springer-Verlag, N.Y. (1994), 269-315. A “Fab fragment” as used herein is comprised of one light chain and the CH1 and variable regions of one heavy chain. The heavy chain of a Fab molecule cannot form a disulfide bond with another heavy chain molecule. An “Fc” region contains two heavy chain fragments comprising the CH2 and CH3 domains of an antibody. The two heavy chain fragments are held together by two or more disulfide bonds and by hydrophobic interactions of the CH3 domains. A “Fab′ fragment” contains one light chain and a portion of one heavy chain that contains the VH domain and the CH1 domain and also the region between the CH1 and C H2 domains, such that an interchain disulfide bond can be formed between the two heavy chains of two Fab′ fragments to form a F(ab′)2 molecule. A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains. The “Fv region” comprises the variable regions from both the heavy and light chains, but lacks the constant regions.

Techniques for the production of antibodies and the elicitation of an immune response against a specific antigen are well known in the art and described, e.g. in Howard and Bethell (2000) Basic Methods in Antibody Production and Characterization, Crc. Pr. Inc.

In particular, antibodies of the present invention include immunoglobulin molecules and immunologically active portions of immunoglobulin molecules, i.e., molecules that contain an antigen binding site that immunospecifically binds to a mutant calreticulin protein (e.g., one or more complementarity determining regions (CDRs) of an anti-mutant calreticulin protein antibody). In a preferred aspect, the antibodies are humanized or human and/or deimmunized. More preferably, the antibodies are humanized and most preferably the antibodies are fully humanized/human.

Said “fully humanized antibody” are also characterized and described as “completely human” or “fully human” antibodies. All these antibodies can be generated by methods known in the art. For example, by phage display technology, recombinant antibody molecules may be generated due to the use of in vitro maturation which is the usage of a complete human immunoglobulin γ, subclass-1 framework (IgG1) as described by Knappik (2000) J. Mol Biol. 296(1), 57-86, and Rauchenberger (2003) J Biol Chem. 278(40), 38194-205.

The present invention also relates to the production of recombinant antibodies. A wide variety of recombinant antibody formats have been developed in the recent past, e.g. bivalent, trivalent or tetravalent bispecific antibodies. Examples include the fusion of an IgG antibody format and single chain domains (for different formats see e.g. Coloma, M. J., et al., Nature Biotech 15 (1997), 159-163; WO 2001/077342; Morrison, S. L., Nature Biotech 25 (2007), 1233-1234; Holliger, P., et. al, Nature Biotech. 23 (2005), 1126-1136; Fischer, N., and Léger, O., Pathobiology 74 (2007), 3-14; Shen, J., et. al., J. Immunol. Methods 318 (2007), 65-74; Wu, C., et al., Nature Biotech. 25 (2007), 1290-1297). The bispecific antibody or fragment herein also includes bivalent, trivalent or tetravalent bispecific antibodies described in WO 2009/080251; WO 2009/080252; WO 2009/080253; WO 2009/080254; WO 2010/112193; WO 2010/115589; WO 2010/136172; WO 2010/145792; WO 2010/145793 and WO 2011/117330. Thus, the present invention also relates to recombinant human antibodies, heterologous antibodies and heterohybrid antibodies. The term “recombinant antibody” includes all sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human and non-human combinatorial antibody library, or antibodies prepared, expressed, created or isolated by any other means that involves splicing of human immunoglobulin gene sequences to other DNA sequences. Such recombinant antibodies have variable and constant regions (if present) derived from germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to germline VH and VL sequences, may not naturally exist within the antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones, Nature 321 (1986), 522-525; Reichmann Nature 332 (1998), 323-327 and Presta Curr Op Struct Biol 2 (1992), 593-596. A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. No. 5,225,539, U.S. Pat. No. 5,693,761 and U.S. Pat. No. 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084). Inventive antibody molecules can easily be produced in sufficient quantities, inter alia, by recombinant methods known in the art, see, e.g. Bentley, Hybridoma 17 (1998), 559-567; Racher, Appl. Microbiol. Biotechnol. 40 (1994), 851-856; Samuelsson, Eur. J. Immunol. 26 (1996), 3029-3034.

Further methods for the production of antibodies are well known in the art, see, e.g. Harlow and Lane, “Antibodies, A Laboratory Manual”, CSH Press, Cold Spring Harbor, 1988.

As used herein, the term “CDR-grafted”, “humanized” or “humanization” are used interchangeably to refer to a human antibody as defined herein (preferably a IgG1 antibody) comprising in its binding domains at least one complementarity determining region (“CDR”) from a non-human antibody or fragment thereof. Humanization approaches are described for example in WO 91/09968 and U.S. Pat. No. 6,407,213. As non-limiting examples, the term encompasses the case in which a variable region of the binding domain comprises a single CDR region, for example the third CDR region (CDR-H3) of the VH, from another non-human animal, for example a rodent, as well as the case in which a or both variable region/s comprise at each of their respective first, second and third CDRs the CDRs from said non-human animal. In the event that all CDRs of a binding domain of the antibody have been replaced by their corresponding equivalents from, for example, a rodent, one typically speaks of “CDR-grafting”, and this term is to be understood as being encompassed by the term “humanized” as used herein. The term “humanized” also encompasses cases in which, in addition to replacement of one or more CDR regions within a VH and/or VL of the binding domain further mutation/s (e.g. substitutions) of at least one single amino acid residue/s within the framework (“FR”) regions between the CDRs has/have been effected such that the amino acids at that/those positions correspond/s to the amino acid/s at that/those position/s in the animal from which the CDR regions used for replacement is/are derived. As is known in the art, such individual mutations are often made in the framework regions following CDR-grafting in order to restore the original binding affinity of the non-human antibody used as a CDR-donor for its target molecule. The term “humanized” may further encompass (an) amino acid substitution(s) in the CDR regions from a non-human animal to the amino acid(s) of a corresponding CDR region from a human antibody, in addition to the amino acid substitutions in the framework regions as described above.

More specifically, as used herein, “humanized antibodies” or related terms encompass antibodies having the amino acid sequence of a human immunoglobulin with a variable region comprising non-human CDR- and/or framework region-sequences. In contemplating an antibody intended for therapeutic administration to humans, it is highly advantageous that the major part of this antibody is of human origin. Following administration to a human patient, a humanized antibody or a human antibody (or fragment thereof) will most probably not elicit a strong immunogenic response by the patient's immune system, i.e. will not be recognized as being a “foreign”, that is non-human protein. This means that no host, i.e. patient antibodies will be generated against the therapeutic antibody which would otherwise block the therapeutic antibody's activity and/or accelerate the therapeutic antibody's elimination from the body of the patient, thus preventing it from exerting its desired therapeutic effect. An antibody as defined herein may also be regarded as humanized if it consists of (a) sequence(s) that deviate(s) from its (their) closest human germline sequence(s) by no more than would be expected due to the imprint of somatic hypermutation. Preferably, the humanized antibodies as defined herein have a human constant region and one or more of the CDR sequences which may be of, but are not limited to, CDRs of non-human, preferably rodent, origin. However, in context of this invention, also antibodies are provided that comprise not only human constant regions but also CDRs that are of human origin. Accordingly, the present invention also provides for “fully-human” antibodies.

As used herein, the term “chimeric antibody” encompasses antibodies having human constant regions on the light and heavy chains and non-human variable regions on the light and heavy chains. Preferably the non-human regions are from a rodent sequence. For example, the variable regions of the heavy and light chain could be amplified by RT-PCR using RNA extracted from a mouse hybridoma cell which produces the antibody of interest. The amplified sequence could be cloned in frame with the constant heavy-chain or the constant light chain respectively of a human IgG also included in a mammalian expression vector. An expression vector encoding a chimeric IgG could be transfected into the right cell line, like for example CHO or HEK293, for chimeric antibody production.

As used herein, the term “deimmunized” or “deimmunization” denotes modification of the binding domain vis-à-vis an original wild type construct by rendering said wild type construct non-immunogenic or less immunogenic in humans. Deimmunization approaches are shown e.g. in WO 00/34317, WO 98/52976, WO 02/079415 or WO 92/10755. The term “deimmunized” also relates to constructs, which show reduced propensity to generate T cell epitopes. In accordance with this invention, the term “reduced propensity to generate T cell epitopes” relates to the removal of T-cell epitopes leading to specific T-cell activation. Furthermore, “reduced propensity to generate T cell epitopes” means substitution of amino acids contributing to the formation of T cell epitopes, i.e. substitution of amino acids, which are essential for formation of a T cell epitope. In other words, “reduced propensity to generate T cell epitopes” relates to reduced immunogenicity or reduced capacity to induce antigen independent T cell proliferation. The term “T cell epitope” relates to short peptide sequences which can be released during the degradation of peptides, polypeptides or proteins within cells and subsequently be presented by molecules of the major histocompatibility complex (MHC) in order to trigger the activation of T cells; see inter alia WO 02/066514. For peptides presented by MHC class II such activation of T cells can then give rise to an antibody response by direct stimulation of T cells to produce said antibodies. “Reduced propensity to generate T-cell epitopes” and/or “deimmunization” may be measured by techniques known in the art. Preferably, de-immunization of proteins may be tested in vitro by T cell proliferation assay. In this assay PBMCs from donors representing >80% of HLA-DR alleles in the world are screened for proliferation in response to either wild type or de-immunized peptides. Ideally cell proliferation is only detected upon loading of the antigen-presenting cells with wild type peptides. Alternatively, one may test deimmunization by expressing HLA-DR tetramers representing all haplotypes. These tetramers may be tested for peptide binding or loaded with peptides substitute for antigen-presenting cells in proliferation assays. In order to test whether deimmunized peptides are presented on HLA-DR haplotypes, binding of e.g. fluorescence-labeled peptides on PBMCs can be measured. Furthermore, deimmunization can be proven by determining whether antibodies against the deimmunized molecules have been formed after administration in patients. Preferably, antibody derived molecules are deimmunized in the framework regions and most of the CDR regions are not modified in order to generate reduced propensity to induce T cell epitope so that the binding affinity of the CDR regions is not affected. Even elimination of one T cell epitope results in reduced immunogenicity. In summary, the above approaches help to reduce the immunogenicity of the antibodies provided herein when being administered to patients.

The invention also involves one or more of the disclosed CDR sequences above or a CDR sequence at least 75% (at least 80%, at least 90%, at least 95%, at least 96%, at least 97%, at least 98% or at least 99%) identical in their amino acid sequence hereto wherein said CDR sequence is in the context of an antibody framework/framework region. Preferably, the antibody framework is a human antibody framework. Examples for frameworks include an IgG framework, such as a murine IgG framework, like IgG1, IgG4, IgG2a and IgG2b, preferably a human IgG framework such as IgG1, IgG2, IgG3 and IgG4. Accordingly, the antibodies of the invention may also comprise cross-cloned antibodies, i.e. antibodies comprising different antibody regions (e.g. CDR-regions) from one or more parental or affinity-optimized antibody(ies) as described herein. These cross-cloned antibodies may be antibodies in several, different frameworks, e.g. an IgG-framework, e.g. a IgG1-, IgG4, IgG2a or an IgG2b-framework. For example, said antibody framework is a mammalian, e.g. a human framework such as IgG1, IgG2, IgG3 or IgG4. It is of note that not only cross-cloned antibodies described herein may be presented in a preferred (human) antibody framework, but also antibody molecules comprising CDRs from antibodies as described herein, may be introduced in an immunoglobulin framework. Examples for frameworks include IgG frameworks such as IgG1, IgG4, IgG2a and IgG2b. Most preferred are human frameworks, and particularly human IgG1 or IgG4 frameworks. IgG4 acts mainly in monovalent form. IgG4 is a slightly modified version of IgG1.

As used herein, a “human antibody framework” relates to an antibody framework that is substantially identical (about 85% or more, usually 90%, more preferably 95%, 96%, 97%, 98%, 99% or more) to the antibody framework of a naturally occurring human immunoglobulin.

As used herein, a “human framework region” relates to a framework region that is substantially identical (about 85% or more, usually 90%, more preferably 95%, 96%, 97%, 98%, 99% or more) to the framework region of a naturally occurring human immunoglobulin.

In accordance with this invention, a framework region relates, accordingly, to a region in the V domain (VH or VL domain) of immunoglobulins and T-cell receptors that provides a protein scaffold for the hypervariable complementarity determining regions (CDRs) that make contact with the antigen. In each V domain, there are four framework regions designated FR1, FR2, FR3 and FR4. Framework 1 encompasses the region from the N-terminus of the V domain until the beginning of CDR1, framework 2 relates to the region between CDR1 and CDR2, framework 3 encompasses the region between CDR2 and CDR3 and framework 4 means the region from the end of CDR3 until the C-terminus of the V domain; see, inter alia, Janeway, Immunobiology, Garland Publishing, 2001, 5th ed. Thus, the framework regions encompass all the regions outside the CDR regions in VH or VL domains. Furthermore, the term “transition sequence between a framework and a CDR region” relates to a direct junction between the framework and CDR region. In particular, the term “transition sequence between a framework and a CDR region” means the sequence directly located N- and C-terminally of the CDR regions or amino acids surrounding CDR regions. Accordingly, frameworks may also comprise sequences between different CDR regions. The person skilled in the art is readily in a position to deduce from a given sequence the framework regions, the CDRs as well as the corresponding transition sequences; see Kabat (1991) Sequences of Proteins of Immunological Interest, 5th edit., NIH Publication no. 91-3242 U.S. Department of Health and Human Services, Chothia (1987). J. Mol. Biol. 196, 901-917 and Chothia (1989) Nature, 342, 877-883.

In a certain aspect, the antibody is an immunoglobulin, for example a human immunoglobulin selected from the group consisting of IgA, IgD, IgE, IgG or IgM antibody, preferably human IgG. As used herein, an “antibody” may denote immunoglobulin molecules, i.e., molecules that contain an antigen binding site that immunospecifically bind to a mutant calreticulin protein. Such antibodies are constructed in the same way. They form paired heavy and light polypeptide chains, and the generic term immunoglobulin is used for all such proteins. Within this general category, however, five different classes of immunoglobulins—IgM, IgD, IgG, IgA, and IgE—can be distinguished by their C regions. IgG antibodies are large molecules, having a molecular weight of approximately 150 kDa, composed of two different kinds of polypeptide chain. One, of approximately 50 kDa, is termed the heavy or H chain, and the other, of approximately 25 kDa, is termed the light or L chain. Each IgG molecule consists of two heavy chains and two light chains. The two heavy chains are linked to each other by disulfide bonds and each heavy chain is linked to a light chain by a disulfide bond. In any given immunoglobulin molecule, the two heavy chains and the two light chains are identical, giving an antibody molecule two identical antigen-binding sites, and thus the ability to bind simultaneously to two identical structures. Two types of light chain, termed lambda and kappa, are found in antibodies. A given immunoglobulin either has lambda chains or kappa chains, never one of each. No functional difference has been found between antibodies having lambda or kappa light chains, and either type of light chain may be found in antibodies of any of the five major classes. The ratio of the two types of light chain varies from species to species. In mice, the average kappa to lambda ratio is 20:1, whereas in humans it is 2:1 and in cattle it is 1:20. The reason for this variation is unknown. By contrast, the class, and thus the effector function of an antibody, is defined by the structure of its heavy chain. There are five main heavy-chain classes or isotypes, some of which have several subtypes, and these determine the functional activity of an antibody molecule such as, for example, complement-dependent cytotoxicity (CDC) and antibody-dependent cell-mediated cytotoxicity (ADCC). The five major classes of immunoglobulin are immunoglobulin M (IgM), immunoglobulin D (IgD), immunoglobulin G (IgG), immunoglobulin A (IgA), and immunoglobulin E (IgE). Their heavy chains are denoted by the corresponding lower-case Greek letter (mu, delta, gamma, alpha, and epsilon, respectively). IgG is by far the most abundant immunoglobulin and has several subclasses (IgG1, 2, 3, and 4 in humans, IgG1, IgG2a, IgG2b and IgG3 in mice). Their distinctive functional properties are conferred by the carboxy-terminal part of the heavy chain, where it is not associated with the light chain. The general structural features of all the isotypes are similar. The IgG antibody is the most abundant isotype in plasma.

Preferably, the antibodies as defined herein are IgG antibodies. As is well known in the art, an IgG comprises not only the variable antibody regions responsible for the highly discriminative antigen recognition and binding, but also the constant regions of the heavy and light antibody polypeptide chains normally present in endogenously produced antibodies and, in some cases, even decoration at one or more sites with carbohydrates. Such glycosylation is generally a hallmark of the IgG format, and portions of these constant regions make up the so called Fc region of a full antibody which is known to elicit various effector functions in vivo, such as e.g. antibody-dependent cellular cytotoxicity (ADCC). In addition, the Fc region mediates binding of the IgG to an Fc receptor, hence prolonging half life in vivo as well as facilitating homing of the IgG to locations with increased Fc receptor presence. Advantageously, the IgG antibody is an IgG1 or IgG4 antibody specifically binding to a mutant calreticulin protein.

In the following exemplary methods for the generation of variants of the antibodies disclosed herein that specifically bind to a mutant calreticulin protein (like monoclonal, humanized, human antibodies or antibody fragments) are described. These variant antibodies, may, for example, bind to the same epitope as the antibodies disclosed or deposited herein.

### Generation of Antibodies to a Mutant Calreticulin Protein

Antibodies and fragments thereof to a mutant calreticulin protein or an epitope thereof (also referred to as a target protein) for therapeutic and/or diagnostic uses can be obtained in any number of ways known to those of ordinary skill in the art. These antibodies can be used in accordance with the invention and/or as the basis of engineering new antibodies. Phage display techniques can be used to generate or isolate an antibody and/or fragment thereof to a mutant calreticulin protein or an epitope thereof. Standard hybridoma technologies can be used to generate antibodies and fragments thereof to a mutant calreticulin protein or an epitope thereof. For example, the antibody or fragment thereof to a mutant calreticulin protein or an epitope thereof is a monoclonal antibody or a fragment thereof. For example, the antibody or fragment thereof to a mutant calreticulin protein or an epitope thereof is a polyclonal antibody or a fragment thereof. For example, the antibody or fragment thereof to a mutant calreticulin protein or an epitope thereof is a recombinant antibody or a fragment thereof. For example, the antibody or fragment thereof to a mutant calreticulin protein or an epitope thereof is a humanized antibody or a fragment thereof. For example, the antibody or fragment thereof to a mutant calreticulin protein or an epitope thereof is a fully human antibody or a fragment thereof. For example, the antibody or fragment thereof to a mutant calreticulin protein or an epitope thereof is a chimeric antibody or fragment thereof. For example, the antibody or fragment thereof (e.g., CDR(s)) to a mutant calreticulin protein or an epitope thereof is derived from an animal source (e.g., mouse, rat, or rabbit).

### Polyclonal Antibodies

The antibodies specifically binding to a mutant calreticulin protein or an epitope thereof can be polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the mutant calreticulin protein (or fragment or epitope thereof) or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

### Monoclonal Antibodies

The antibodies specifically binding to a mutant calreticulin protein or an epitope thereof may, be monoclonal antibodies and/or fragments thereof. Monoclonal antibodies may be prepared using known hybridoma methods, such as those described by Kohler and Milstein (1975) Nature 256:495. In a hybridoma method, a mouse, hamster, or other appropriate host animal (e.g., rabbit, goat etc.), is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the to a mutant calreticulin protein or an epitope thereof (or fragment thereof or an epitope thereof) or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor (1984) Immunol. 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against target protein. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980) Anal. Biochem. 107:220.

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, HEK293 cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (e.g., U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies and fragments thereof may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

### Human and Humanized Antibodies

The antibodies of the invention that specifically bind to a mutant calreticulin protein may further comprise humanized antibodies or human antibodies (and/or fragments thereof). Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies (and/or fragments thereof) include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies (and/or fragments thereof) may also comprise residues which are found neither in the recipient antibody nor in the imported CDR or framework sequences. In general, the humanized antibody (and/or fragments thereof) will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al. (1986) Nature, 321:522-525; Riechmann et al. (1988) Nature 332:323-329; and Presta (1992) Curr. Op. Struct. Biol. 2:593-596).

Methods for humanizing non-human antibodies (and/or fragments thereof) are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature, 321:522-525; Riechmann et al. (1988) Nature 332:323-327; Verhoeyen et al. (1988) Science 239:1534-1536), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies (and/or fragments thereof) can also be produced using various techniques known in the art, including phage display libraries (Hoogenboom and Winter (1991) J. Mol. Biol. 227:381; Marks et al. (1991) J. Mol. Biol. 222:581). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al. (1991) J. Immunol. 147(1):86-95). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783; Lonberg et al. (1994) Nature 368:856-859; Morrison (1994) Nature 368:812-13; Fishwild et al. (1996) Nature Biotechnology 14:845-51; Neuberger (1996) Nature Biotechnology 14:826; Lonberg and Huszar (1995) Intern. Rev. Immunol. 13 65-93.

The antibodies (and/or fragments thereof) may also be affinity matured using known selection and/or mutagenesis methods as described above. Preferred affinity matured antibodies have an affinity which is 5 times, more preferably 10 times, even more preferably 20 or 30 times greater than the starting antibody (generally murine, humanized or human) from which the matured antibody is prepared.

### Antibody Fragments

Various techniques have been developed for the production of antibody fragments. Traditionally, these fragments were derived via proteolytic digestion of intact antibodies (see Morimoto et al (1992) Journal of Biochemical and Biophysical Methods 24:107-117; and Brennan et al (1985) Science 229:81). Antibody fragments can also be produced directly by recombinant host cells and the antibody phage libraries discussed above. Fab′-SH fragments can be directly recovered from E. coli and chemically coupled to form F(ab′)2 fragments (Carter et al (1992) Bio/Technology 10:163-167). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870, for example. Such linear antibody fragments may be monospecific or bispecific.

### Multispecific and Bispecific Antibodies

Bispecific antibodies with binding specificities for at least two different epitopes (Millstein et al (1983), Nature 305:537-539) may bind to two different epitopes of the mutant calreticulin protein. An anti-mutant calreticulin protein arm may be combined, for example, with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the mutant calreticulin protein-expressing cell. Bispecific antibodies may also be used to localize cytotoxic agents to cells which express mutant calreticulin protein (WO 96/16673; U.S. Pat. No. 5,837,234; WO98/02463; U.S. Pat. No. 5,821,337). Purification methods for bispecific antibodies have been disclosed (WO 93/08829; Traunecker et al (1991) EMBO J. 10:3655-3659; WO 94/04690; Suresh et al (1986) Methods in Enzymology 121:210; U.S. Pat. No. 5,731,168). Bispecific antibodies can be produced using leucine zippers (Kostelny et al (1992) J. Immunol. 148(5):1547-1553), and single-chain Fv (sFv) dimers (Gruber et al (1994) J. Immunol. 152:5368).

Techniques for generating bispecific antibodies from antibody fragments have also been described, such as using chemical linkage wherein intact antibodies are proteolytically cleaved to generate F(ab′)2 fragments (Brennan et al (1985) Science 229:81). Fab′-SH fragments can be recovered from E. coli and chemically coupled to form bispecific antibodies (Shalaby et al (1992) J. Exp. Med. 175:217-225. The “diabody” technology provides an alternative method for making bispecific antibody fragments (Hollinger et al (1993) Proc. Natl. Acad. Sci. USA 90:6444-6448).

Antibodies with more than two valencies are contemplated. Multivalent, “Octopus” antibodies with three or more antigen binding sites and two or more variable domains can be readily produced by recombinant expression of nucleic acid encoding the polypeptide chains of the antibody (US 2002/0004586; WO 01/77342). For example, trispecific antibodies can be prepared (Tuft et al (1991) J. Immunol. 147:60.)

### Conjugated Antibodies

The antibody specifically binding to a mutant calreticulin protein can be conjugated to one or more therapeutic agents. This is particularly envisaged when the antibodies are to be used in medicine, for example, in the therapy/treatment of a myeloid malignany. The therapeutic agent(s), such as toxin(s), are preferably suitable for the treatment of myeloid malignancies.

Antibody conjugates with antibodies to a mutant calreticulin protein can prepared for various types of antibodies (and/or fragments thereof) including chimeric antibodies, humanized antibodies, and fully human antibodies. As used herein, “conjugated” means that the antibody/binding molecule is bound to the therapeutic agent(s) via any type of bonding, and thus includes bonding via fusion proteins (in case the therapeutic agent is of peptidic nature) or any other type of coupling or linkage between the therapeutic agent and the antibody/binding molecule. “Conjugated to a therapeutic agent” is thus to be understood as including fused to, linked to or coupled to a therapeutic agent.

“Therapeutic agent” as used herein refers to any molecule (including small molecules, macromolecules, peptides, polypeptides, proteins (including other therapeutic antibodies), radioactive isotopes, etc) exerting a beneficial effect in the treatment of diseases in humans or other mammals. The term “therapeutic agents” also comprises toxins,

A molecule of antibody may conjugate with more than one molecule of the therapeutic agent (as used herein, “conjugation agent”), depending on the number of sites in the antibody available for conjugation and the experimental conditions employed for performing the conjugation. As it will be apparent to those skilled in the art, while each molecule of antibody is conjugated to an integer number of molecules of the conjugation agent, a preparation of the antibody conjugate may analyze for a non-integer ratio of conjugation agent molecules per molecule of antibody, reflecting a statistical average.

Examples of therapeutic agents that can be conjugated to the antibodies/binding molecules of the invention include, but are not limited to, anticancer agents such as antimetabolites (e.g., methotrexate, azathioprine, 6-mercaptopurine, 6-thioguanine, cytarabine, 5-fluorouracil, capecitabine and decarbazine), alkylating agents (e.g., mechlorethamine, thiotepa chlorambucil, melphalan, carmustine (BCNU), lomustine (CCNU), cyclophosphamide, ifosfamide, busulfan, dibromomannitol, streptozotocin, mitomycin C, cis-dichlorodiamine platinum (II) (DDP) cisplatin, carboplatin, oxaliplatin nedaplatin, satraplatin, triplatin tetranitrate, procarbazine, altretamine, and tetrazines), anthracyclines (e.g., daunorubicin, doxorubicin, valrubicin, idarubicin, epirubicin and mitoxantrone), antibiotics (e.g., actinomycins like dactinomycin, bleomycins, mithramycins, calicheamicins, mitomycins, duocarmycins and anthramycins (AMC)), topoisomerase inhibitors (e.g. irinotecan, topotecan, camptothecin, etoposide and teniposide), and anti-mitotic agents (e.g., vinca alkaloids such as vincristine, vinorelbine, vindesine and vinblastine, taxanes such as paclitaxel (or taxol) and docetaxel, and other tubulin polimeryzation inhibitors such as auristatins like monomethyl auristatin E (MMAE) and monomethyl auristatin F (MMAF) and maytansine derivatives (a.k.a maytansinoids) like mertansine (also known as DM1) and DM4). The term “anticancer agent” as used herein refers to and includes cytotoxic agents.

Other therapeutic agents that can be conjugated to the antibodies of the invention include toxins and inhibitory peptides. As used herein, “inhibitory peptide” means any peptide that inhibits cell proliferation or affects cell viability via any mechanism of action. Non-limiting examples are provided herein below.

Specific examples of anticancer agents that can be conjugated to the antibodies/binding molecule of the invention include, but are not limited, to taxol, cytochalasin B, gramicidin D, ethidium bromide, emetine, mitomycin, etoposide, tenoposide, irinotecan, topotecan, camptothecin, vincristine, vinblastine, colchicin, doxorubicin, daunorubicin, dihydroxy anthracin dione, mitoxantrone, mithramycin, calicheamicin, duocarmycin, actinomycin D, glucocorticoids, monomethyl auristatin E (MMAE), monomethyl auristatin F (MMAF), maytansine derivatives like mertansine (also known as DM1) and DM4, and puromycin and analogs or homologs thereof.

Specific examples of inhibitory peptides that can be conjugated to the antibodies/binding molecule of the invention include but are not limited to the following peptide sequences:
YARAAARQARAGRGYVSTT (wherein Y represents a phosphotyrosine), which is a peptide inhibitor of the transcription factor STAT6 which binds only to the phosphorylated form of STAT6 to prevent its dimerization and activity
PYLKTK (wherein Y represents a phosphotyrosine), which is a phosphopeptide which inhibits the activity of the transcription factor STAT3 in vitro and in vivo
MVRRFLVTLRIRRACGPPRVRV, which is part of the n-terminal sequence of p14ARF and it is able to induce apoptosis.

In one aspect, the therapeutic agent for conjugation is a toxin. The toxin can be an enzyme. Specific examples of toxins that can be conjugated to the antibodies/binding molecules of the invention include, but are not limited to plant toxins such as saporin, Ricin or Gelonin, and bacterial toxins such as Pseudomona exotoxin or diphteria toxin, and derivatives thereof. Also, ribonucleases can be considered as toxins due to their ability to degrade RNA and cause cell death. Some Rnases which are considered to have cytotoxic effects and can be used also as toxins are Binase (from Bacillus intermedius), α-sarcin (from Aspergillus giganteus), Ranpirnase (from amphinian), Onconase (from Rana pipiens), and human RNAses like inhibitor-resistant variant of human pancreatic RNase (HP-DDADD-RNase)

The antibodies/binding molecules of the invention may also be conjugated to nanoparticles comprising human serum albumin (HSA) to optimize preparation and uptake of antibodies in cancer cells, as described, for example, by Steinhauser et al., Biomaterials 2006 October; 27(28):4975-83.

Such antibody conjugates with antibodies/binding molecules to a mutant calreticulin protein can readily be prepared for various types of antibodies (and/or fragments thereof) as described above, including chimeric antibodies, deimmunized antibodies, humanized antibodies, fully humanized/human antibodies, single chain antibodies, diabodies and the like. Techniques for conjugating agents, such as the therapeutic agents described above, to antibodies are well known (see, e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates,” Immunol. Rev., 62:119-58 (1982)). Conjugates can be prepared using a variety of cleavable linkers such as for example disulfide-based linkers, hydrazone linkers or peptide linkers (Alley et al. (2010) Curr Opin Chem Biol 14(4):529-37; Webb (2011) Nat. Biotech, 29(4):297-8) or the TAP linker technology from ImmunoGen. Alternatively, the conjugate may be prepared via fusion proteins, as disclosed below.

The antibodies of the invention may also be a fusion wherein the antibody portion (comprising one or more CDRs) is fused to another protein or polypeptide. For example, an antibody according to the invention can be fused to another protein or polypeptide wherein said protein or polypeptide is an agent which improves the properties of said antibody e.g., enhances therapeutic effect. Such proteins or polypeptides which e.g., can enhance therapeutic effect through a number of mechanisms like attracting or enhancing an immune response or delivering a therapeutic agent such a cytotoxic peptide or inhibitory peptide as defined above. Examples of such proteins or polypeptides are cytokines like IL2 or a IL2 homolog or GM-CSF. A nucleic acid encoding the antibody of the invention operably linked to the desired protein or polypeptide can be prepared and introduced into a suitable expression vector, which is then inserted into a host cell for production of the fusion protein.

The antibodies (and fragments thereof) of the invention can also be conjugated to or have a detectable label to molecules for diagnostic purposes. For example, an antibody to mutant calreticulin protein can be conjugated to a detectable label (e.g., for imaging purposes) for diagnosing or detecting a myeloid malignancy. Suitable detectable markers include, but are not limited to, a radioisotope, a nanoparticle, a fluorescent compound, a bioluminescent compound, chemiluminescent compound, a metal chelator or an enzyme. Techniques for conjugating diagnostic agents to antibodies are well known (Holmes et al. (2001) Curr Protoc Cytom. May; Chapter 4:Unit 4.2; Kumar et al (2008) ACS Nano. March; 2(3):449-56; Rosenthal et al. (2006) Laryngoscope September; 116(9):1636-41). Additionally kits for conjugating agents (such as a detectable label) to diagnostic antibodies are commercially available.

In a certain aspect, the present invention relates to a nucleic acid molecule having a sequence encoding the antibody as defined and provided herein. The nucleic acid molecules of the invention, for example, those encoding anti-mutant calreticulin protein antibodies, and its subsequences/alternative transcripts, can be inserted into a vector, which will facilitate expression of the insert. The nucleic acid molecules and the antibodies they encode can be used directly or indirectly as therapeutic (or diagnostic) agents (directly in the case of the antibody or indirectly in the case of a nucleic acid molecule). Accordingly, the present invention relates to a vector comprising the nucleic acid molecule encoding the anti-mutant calreticulin protein antibodies. The vector may further comprise a nucleic acid molecule having a regulatory sequence which is operably linked to the nucleic acid molecule encoding the anti-mutant calreticulin protein antibodies. The vector may be an expression vector. Further, the present invention relates to a host, host cell or host cell line transformed or transfected with the vector as defined above. In other words, the host, host cell or host cell line expresses the antibody as provided herein. Said host, host cell or host cell line can be prokaryotic or eukaryotic. The host is preferably a eukaryotic host cell like COS, CHO, HEK293 or a multiple myeloma host cell.

In one embodiment, the present invention provides a hybridoma 8B2-H6-10.7 deposited under accession number DSM ACC3249 with the depositary institute DSMZ (Braunschweig, Germany) on Sep. 12, 2014. The term “8B2-H6-10.7” refers to the herein used designation of the hybridoma. In one aspect, the present invention provides a hybridoma deposited under accession number DSM ACC3249 with the depositary institute DSMZ (Braunschweig, Germany) on Sep. 12, 2014.

The antibody of the invention can be made by any number of methods. For example, the antibody can be synthesized in a cell line harboring a nucleic acid encoding the antibody as described above and culturing said cell line under conditions sufficient to allow expression of said antibody. Accordingly, the present invention relates in one embodiment to a process for the production of the antibody as defined herein, said process comprising culturing a host as defined herein under conditions allowing the expression of the antibody and recovering the produced antibody from the culture. The antibody thus obtained can then be conjugated to a therapeutic agent or to a detectable label for diagnostic purposes, as described above. In the event the antibody is conjugated to a protein (for example a marker or label protein or a therapeutic or a toxic protein) via a fusion protein, a vector encoding the sequence for the fusion protein would be incorporated into the host cell line, which would then be cultured as described above. Techniques for producing and purifying antibodies are well known (see e.g. Liu et al. (2010) MAbs. 2(5):480-99; Shukla et al. (2010) Trends Biotechnol. 28(5):253-61; and Backliwal et al. (2008) Nucleic Acids Res. 36(15):e96).

A “recombinant host” may be any prokaryotic or eukaryotic cell that contains a cloning vector, expression vector, or other heterologous nucleic acid sequences. This term also includes those prokaryotic or eukaryotic cells that have been genetically engineered to contain the cloned gene(s) in the chromosome or genome of the host cell

A “host cell” is a transformed cell or a transfected cell that contains an expression vector and supports the replication or expression of the expression vector. Host cells may be cultured cells, explants, cells in vivo, and the like. Host cells may be prokaryotic cells, for example, E. coli, or eukaryotic cells, for example, yeast, insect, amphibian, or mammalian cells, for example, Vero, CHO, HEK293, HeLa, and others.

As used herein, the term “transformed (host) cell” or “transfected (host) cell” (and the like) means a cell into which (or into predecessor or an ancestor of which) a nucleic acid molecule encoding an antibody (or a fragment thereof) of the invention has been introduced, by means of, for example, recombinant DNA techniques or viruses.

An “isolated DNA molecule” is a fragment of DNA that has been separated from the chromosomal or genomic DNA of an organism. Isolation also is defined to connote a degree of separation from original source or surroundings.

“Complementary DNA” (cDNA), often referred to as ““copy DNA”, is a single-stranded DNA molecule that is formed from an mRNA template by the enzyme reverse transcriptase. Those skilled in the art also use the term “cDNA” to refer to a double-stranded DNA molecule that comprises such a single-stranded DNA molecule and its complement DNA strand.

The term “expression” refers to the biosynthesis of a gene product, such as a protein or an mRNA molecule.

An “expression vector” is a nucleic acid construct, generated recombinant or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene or coding region is said to be “operably linked to” or “operatively linked to” or “operably associated with” the regulatory elements, meaning that the gene or coding region is controlled or influenced by the regulatory element.

The terms “isolated”, “purified” or “biologically pure” refer to material that is free to varying degrees from components which normally accompany it as found in its native state. The antibodies provided herein (as well as the nucleic acids encoding them, the herein provided vectors and hosts) are preferably “isolated”, “purified” or “biologically pure” as defined herein. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or antibody of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or antibody gives rise to essentially one band in an electrophoretic gel. For an antibody that can be subjected to modifications, for example, phosphorylation or glycosylation, different modifications may give rise to different isolated proteins, which can be separately purified. Various levels of purity may be applied as needed according to this invention in the different methodologies set forth herein. The customary purity standards known in the art may be used if no standard is otherwise specified.

An “isolated nucleic acid molecule” can refer to a nucleic acid molecule, depending upon the circumstance, which is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

“Nucleic acid” can refer to deoxyribonucleotides or ribonucleotides and polymers thereof in either single- or double-stranded form. The term can encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively, modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al. (1991) Nucleic Acid Res, 19:081; Ohtsuka et al., J. Biol. Chem. (1985) 260:2600-2608 Rossolini et al. (1994) Mol. Cell Probes, 8:91-98). The term nucleic acid can be used interchangeably with gene, cDNA, mRNA, oligonucleotide, and polynucleotide.

In a certain aspect, the present invention relates to an antibody that specifically binds to a mutant calreticulin protein, wherein said antibody is obtained or obtainable from hybridoma 8B2-H6-10.7 deposited under accession number DSM ACC3249 with the depositary institute DSMZ on Sep. 12, 2014.

In a certain aspect, the present invention provides an antibody, prepared by a process comprising

culturing hybridoma 8B2-H6-10.7 deposited under accession number DSM ACC3249 with the depositary institute DSMZ on Sep. 12, 2014, under conditions that provide for the production of the antibody by the hybridoma; and
recovering of the antibody from the culture.

As discussed herein above, the present invention also relates to anti-mutant calreticulin protein binding molecules/antibodies that comprise CDRs and/or variable regions and/or chains that are at least 75% identical (e.g. 80%, more preferably 85%, 90%, most preferably 95%, 96%, 97%, 98%, 99% or more) to the amino acid sequence of these (individual) CDRs or said variable regions or said chains disclosed herein or as obtainable from hybridoma 8B2-H6-10.7 deposited under accession number DSM ACC3249 with the depositary institute DSMZ on Sep. 12, 2014. Accordingly, the methods of preparation of these binding molecules/antibodies are also provided herein and as laid down herein above.

It is evident that the present invention also relates to antibody/binding molecules that show in their amino acid sequences of their individual CDRs and/or their variable regions and/or chains at least 75% identity (e.g. 80%, more preferably 85%, 90%, most preferably 95%, 96%, 97%, 98%, 99% or more) to the antibody molecules/binding molecules defined herein by their sequences as obtainable from hybridoma 8B2-H6-10.7 deposited under accession number DSM ACC3249 with the depositary institute DSMZ on Sep. 12, 2014. Therefore, the present invention also relates to antibodies/binding molecules that bind to and/or recognize the same epitope on the mutant calreticulin protein and/or that have the same functional properties as the antibodies/binding molecules obtainable from the hybridoma 8B2-H6-10.7 deposited under accession number DSM ACC3249 with the depositary institute DSMZ on Sep. 12, 2014.

The ability of an antibody or binding molecule to bind specifically to mutant calreticulin protein can be determined using well known assays. Affinity or specificity can be determined experimentally by methods known in the art such as Flow Cytometry (FC), Western blots, ELISA-, RIA-, ECL-, IRMA-tests and peptide scans.

The sequences of an antibody provided and to be used in accordance with the present invention, wherein the antibody specifically binds to a mutant calreticulin protein, can be retrieved from hybridoma 8B2-H6-10.7 deposited under accession number DSM ACC3249 with the depositary institute DSMZ on Sep. 12, 2014. The person skilled in the art is readily in a position to isolate the coding nucleic acid molecules from Hybridoma 8B2-H6-10.7 deposited under accession number DSM ACC3249 with the depositary institute DSMZ on Sep. 12, 2014. Routine methods that can be used are known in the art, e.g. in Sambrook “Molecular Cloning: A Laboratory Manual”, Cold Spring Harbor Laboratory.

The following exemplary protocol can be applied to retrieve the nucleic acid sequences of the heavy and light chains of the antibody. A ‘blast analysis’ can be performed with the nucleic acid sequences obtained against appropriate and known databases e.g. the IMGT database. The IMGT database can provide the corresponding amino acid sequence in the appropriate reading frame from the germ line antibody sequences. This database can also provide information regarding the framework region and the CDR (complementarity determining region) of the corresponding antibody, for both heavy and light chains.

The RNA from hybridoma 8B2-H6-10.7 deposited under accession number DSM ACC3249 with the depositary institute DSMZ on Sep. 12, 2014 can be extracted and cDNA can be prepared.

Primers from the Mouse IgG Library primer set (Progen) can be used to amplify the variable regions of the specific immunoglobulin heavy chain and light chain produced by this clone.

The amplification can be performed using primers from the Mouse IgG Library primer set (Progen). Corresponding primers are provided in the table below.

| | |
|---|---|
| H2 | Forward primer A-GAT GTG AAG CTT CAG GAG TC (SEQ ID NO: 22) |
| | Forward primer B-CAG GTG CAG CTG AAG GAG TC (SEQ ID NO: 23) |
| | Reverse primer M-GGC CAG TGG ATA GTC AGA TGG GGG TGT CGT TTT |
| | GGC (SEQ ID NO: 24) |
| H1 | Forward primer C-CAG GTG CAG CTG AAG CAG TC (SEQ ID NO: 25) |
| | Forward primer E-GAG GTG CAG CTG CAA CAA TCT (SEQ ID NO: 26) |
| | Forward primer F-GAG GTC CAG CTG CAG CAG TC (SEQ ID NO: 27) |
| | Forward primer G-CAG GTC CAA CTG CAG CAG CCT (SEQ ID NO: 28) |
| | Forward primer L-GAG GTG CAG CTG GAG GAG TC (SEQ ID NO: 29) |
| | Reverse primer M-GGC CAG TGG ATA GTC AGA TGG GGG TGT CGT TTT |
| | GGC (SEQ ID NO: 30) |
| L1 | Forward primer N-GAT GTT TTG ATG ACC CAA ACT (SEQ ID NO: 31) |
| | Forward primer R-GAC ATT GTG ATG ACC CAG TCT (SEQ ID NO: 32) |
| | Forward primer T-GAT ATC CAG ATG ACA CAG ACT (SEQ ID NO: 33) |
| | Reverse primer X-GGA TAC AGT TGG TGC AGC ATC (SEQ ID NO: 34) |

Specifically, primer pairs A/B+M can be used to generate the H2 heavy chain; primer pairs C/E/F/G/L+M can be used to generate the H1 heavy chain; and primer pairs N/R/T+X can be used to generate the L1 light chain. The forward primers can be used separately in individual reactions together with one respective reverse primer.

The amplification can be performed on cDNA using the AmpliTaq Gold 360 Master Mix (annealing temperature 55° C.) according to the manufacturer's recommendation.

The PCR product can be sequenced. Sequencing can be done with the same set of primers that can be used for PCR amplification.

In a certain aspect, the present invention relates to an antibody that specifically binds to a mutant calreticulin protein,

wherein the antibody is encoded by a nucleic acid molecule that can be obtained by
extraction of RNA from hybridoma 8B2-H6-10.7 deposited under accession number DSM ACC3249 with the depositary institute DSMZ on Sep. 12, 2014;
generation of cDNA using said RNA as a template;
PCR amplification of said cDNA using
   a. a primer pair comprising a 5′-primer as shown in SEQ ID NO: 22 or 23 and a 3′-primer as shown in SEQ ID NO: 24 for amplification of a nucleic acid molecule encoding a heavy chain; and/or
   b. a primer pair comprising a 5′-primer as shown in SEQ ID NO: 31, 32 or 33 and a 3′-primer as shown in SEQ ID NO: 34 for amplification of a light chain.

In a certain aspect, the present invention relates to an antibody that specifically binds to a mutant calreticulin protein,

wherein the antibody is encoded by a nucleic acid molecule that can be obtained by
extraction of RNA from hybridoma 8B2-H6-10.7 deposited under accession number DSM ACC3249 with the depositary institute DSMZ on Sep. 12, 2014;
generation of cDNA using said RNA as a template;
PCR amplification of said cDNA using
   a. a primer pair comprising a 5′-primer as shown in SEQ ID NO: 25, 26, 27, 28, or 29 and a 3′-primer as shown in SEQ ID NO: 24 for amplification of a nucleic acid molecule encoding a heavy chain; and/or
   b. a primer pair comprising a 5′-primer as shown in SEQ ID NO: 31, 32 or 33 and a 3′-primer as shown in SEQ ID NO: 34 for amplification of a light chain.

There herein provided antibodies that specifically bind to mutant calreticulin protein bind to a specific epitope. The antibody provided herein and/or obtainable from the hybridoma 8B2-H6-10.7 deposited under accession number DSM ACC3249 with the depositary institute DSMZ on Sep. 12, 2014 has been generated using the mutant sequence RRKMSPARPRTSCREACLQGWTEA. Accordingly, the antibodies of the present invention bind to RRKMSPARPRTSCREACLQGWTEA or a fragment thereof or an epitope thereof.

The term “binding to an epitope”, does not only relate to a linear epitope but may also relate to a conformational epitope, a structural epitope or a discontinuous epitope consisting of two regions of the mutant calreticulin protein, in particular the C-terminal part thereof, or a fragment thereof. In the context of this invention, a conformational epitope is defined by two or more discrete parts separated in the mutant calreticulin protein, in particular the C-terminal part thereof. Accordingly, specificity can be determined experimentally by methods known in the art and methods as described herein. Such methods comprise, but are not limited to Western Blots, ELISA-, RIA-, ECL-, IRMA-tests and peptide scans.

In a certain aspect, the invention provides compositions comprising an antibody/binding molecule as disclosed herein or having essentially the same biological activity (like binding to the same epitope) of an antibody/binding molecule obtained or obtainable from hybridoma 8B2-H6-10.7 deposited under accession number DSM ACC3249 with the depositary institute DSMZ on Sep. 12, 2014. In a certain aspect, the present invention relates to a composition comprising the antibody/binding molecule directed against/specifically binding to a mutant calreticulin protein as defined herein or as produced by the above described process, a nucleic acid molecule as described herein, a vector as described herein, a host as described herein and/or the deposited hybridoma as disclosed herein.

There herein provided antibodies can be used in the diagnosis of a myeloid malignancy or in the therapy of a myeloid malignancy. A myeloid malignancy is, for example, a myeloproliferative neoplasm or a myelodysplastic syndrome. The myeloproliferative neoplasm can be primary myelofibrosis (PMF) or essential thrombocythemia (ET). The myelodysplastic syndrome can be refractory anemia with ringed sideroblasts and thrombocythemia (RARS-T).

In a certain aspect, the invention provides diagnostic compositions comprising an antibody/binding molecule as disclosed herein or having essentially the same biological activity (like binding to the same epitope) of an antibody/binding molecule obtained or obtainable from hybridoma 8B2-H6-10.7 deposited under accession number DSM ACC3249 with the depositary institute DSMZ on Sep. 12, 2014. In a certain aspect, the present invention relates to a diagnostic composition comprising the antibody/binding molecule directed against/specifically binding to a mutant calreticulin protein as defined herein or as produced by the above described process, a nucleic acid molecule as described herein, a vector as described herein, a host as described herein and/or the deposited hybridoma as disclosed herein.

The diagnostic composition can further comprise, optionally, means and methods for detection. In accordance with the present invention, suitable detectable labels or markers include, but are not limited to, a radioisotope, a nanoparticle, a fluorescent compound, a bioluminescent compound, chemiluminescent compound, a metal chelator or an enzyme. In general, a “label” or a “detectable moiety” is a compound that when linked with the antibody of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

The usefulness of the antibodies/binding molecules that specifically bind to a mutant calreticulin protein in the diagnosis of a myeloid malignancy and/or increased risk for developing a myeloid malignancy can be confirmed as follows:

A cohort of subjects is identified and a sample collected from each subject. The sample is tested for levels of mutant calreticulin protein using the antibodies or fragments thereof mutant calreticulin protein. All subjects may be further tested for the presence of a myeloid malignancy using techniques standard in the art. All subjects may be followed and periodically tested using the inventive antibodies/binding molecules or fragments thereof and further tested for the presence of a myeloid malignancy using techniques standard in the art. After each round of testing, the levels of mutant calreticulin protein are correlated with the presence of a myeloid malignancy and/or increased risk for developing a myeloid malignancy

In a certain aspect, the present invention relates to the use of the antibody as defined and provided herein, the antibody as produced by the herein above described process, the nucleic acid molecule as described above, the vector as described herein, the host and/or the hybridoma as described herein for the preparation of a diagnostic composition, that is, in particular, useful for the diagnosis of a myeloid malignancy. Preferably, the present invention relates to the use of the antibody as defined and provided herein for the preparation of a diagnostic composition that is, in particular, useful for the diagnosis of a myeloid malignancy.

In a certain aspect, the present invention relates to a method for diagnosing a myeloid malignancy, comprising detecting or assaying a mutant calreticulin antibody in a biological sample of an individual suspected of suffering from a myeloid malignancy or suspected of being prone to suffering from a myeloid malignancy using the antibody provided herein, in particular the antibody conjugated with a detectable label as described above. Preferably, the method is an in vitro method. The terms “diagnosing a myeloid malignancy” and “assessing whether a patient/subject suffers from a myeloid malignancy or whether a patient/subject is prone to suffering from a myeloid malignancy” can be used interchangeably herein.

In a certain aspect, the present invention relates to the antibody/binding molecule as defined and provided herein, the antibody as produced by the herein described process, the nucleic acid molecule as described herein, the vector as described herein, the host, the hybridoma and/or the composition as described herein for use in the diagnosis of a myeloid malignancy. Preferably, the present invention relates to the antibody as defined and provided herein for use in the diagnosis of a myeloid malignancy.

In one aspect, the present invention relates to the use of the antibody/binding molecule as defined and provided herein, the antibody/binding molecule as produced by the herein described process, the nucleic acid molecule as described herein, the vector as described herein, the host, the hybridoma and/or the composition as described herein for the preparation of a diagnostic kit for the diagnosis of a myeloid malignancy.

The phrase “detecting a myeloid malignancy” or “diagnosing a myeloid malignancy” refers to determining the presence or absence of a myeloid malignancy in an subject, preferably in a human. “detecting a myeloid malignancy” or diagnosing a myeloid malignancy” also can refer to obtaining indirect evidence regarding the likelihood of the presence of a myeloid malignancy in the subject or assessing the predisposition of a subject to the development of diagnosing a myeloid malignancy”. Detecting a myeloid malignancy can be accomplished using the methods of this invention alone, in combination with other methods, or in light of other information regarding the state of health of the subject.

In a further aspect, the present invention relates to a kit comprising the antibody as provided and described herein, the antibody as produced by the herein described process, the nucleic acid molecule as described herein, the vector as described herein, the host, the hybridoma and/or the composition as described herein. Preferably, the kit comprises the antibody as provided and described herein. Preferably, the kit is used for the diagnosis of a myeloid malignancy.

The kit (to be prepared in context) of this invention or the methods and uses of the invention may further comprise or be provided with (an) instruction manual(s). For example, said instruction manual(s) may guide the skilled person (how) to diagnose of a myeloid malignancy in accordance with the present invention. Particularly, said instruction manual(s) may comprise guidance to use or apply the herein provided methods or uses. The kit (to be prepared in context) of this invention may further comprise substances/chemicals and/or equipment suitable/required for carrying out the methods and uses of this invention. For example, such substances/chemicals and/or equipment are solvents, diluents and/or buffers for stabilizing and/or storing (a) compound(s) required for specifically determining the protein expression level mutant calreticulin as defined herein.

The determination of the presence of mutant calreticulin, in particular of the C-terminus thereof, as described herein can be performed as a stand-alone analysis. Alternatively, this analysis can be followed or preceded by the analysis of other markers for myeloid malignancies, such as JAK2 and MPL mutations. Also simultaneous determination of such markers is envisaged, like the simultaneous test for JAK2 mutation(s) and mutant calreticulin protein (and, optionally, further markers), or the simultaneous test of JAK2 mutation(s), mutant calreticulin and MPL mutation(s) (and, optionally, further markers).

Accordingly (a) kit(s) (or uses of such kits) is/are envisaged herein that provide means for such subsequent or simultaneous tests. For example, said kit may comprise, in addition to (a) compound(s) required for specifically determining the presence (or amount) of one or more mutant calreticulin proteins (or of a gene product thereof), (a) compound(s) required for specifically determining the presence as JAK2 and/or MPL mutations (and optionally further markers), e.g. (a) antibody(ies), (a) (nucleotide) probe(s), (a) primer(s) (pair(s)), (an) antibody(ies) and/or (an) aptamer(s) specific that allow the specific detection of JAK2 and MPL mutations (and optionally further markers).

The CALR mutations cause a frameshift of the translated polypeptide, a characteristic C-terminal amino acid sequence is present in the mutated calreticulin proteins as described and provided herein. This characteristic amino acid sequence alters the overall charge of the protein. It also alters the migration of the mutated calreticulin during protein electrophoresis. One can take advantage of this difference in charge and/or in migration behaviour in order to determine the presence of a mutated calreticulin protein. For example, antibodies specific to mutant calreticulin protein can be used to identify said mutant protein e.g. by Western immunoblotting. Optionally, also antibodies specific to the wild type calreticulin protein can be used (in addition) as a control.

Mutant calreticulin proteins using the herein provided antibodies can be analyzed by methods that include immunologic methodologies, such as immunohistochemistry (IHC), immunocytochemistry, Western blot, ELISA immunoassay, gel- or blot-based methods, mass spectrometry, flow cytometry, or fluorescent activated cell sorting (FACS). Many methods monitor the binding of an antibody or set of antibodies to a protein of interest that detect differences between a wild type and mutant forms. Mass spectrometry detects differences in the size of a protein and its fragments that reveal information about the underlying sequence. Samples that can be assayed/used can be a bone marrow sample, a blood sample or a saliva sample. The sample is preferably a blood sample. The blood sample preferably comprises peripheral granulocytes. The sample can be obtained from a patient by routine techniques, for example, by biopsy.

In the present application it was surprisingly shown that the herein provided antibody was able to specifically bind to mutant calreticulin in an FACS assay using mutant calreticulin expressing cells; see Example 1 and FIG. 8. This indicates that mutant calreticulin protein is localized on the cell surface/present on the extracellular side of the plasma membrane. Due to its presence on the cellular surface, mutant calreticulin can be used as a cell surface marker using e.g. cells expressing mutant calreticulin and/or patient samples containing whole/living/intact cells (like blood samples or bone marrow samples). For example, patient samples containing whole/living cells can be used in the diagnosis of myeloid malignancies, like for example in the diagnosis of meyloproliferative neoplasms like primary myelofibrosis (PMF) or essential thrombocythemia (ET) or in the diagnosis of a myelodysplastic syndrome, like refractory anemia with ringed sideroblasts and thrombocythemia (RARS-T) using the herein provided antibodies. Any assays that allow the analysis of such samples (e.g. patient samples containing whole/living/intact cells) can be used herein. Preferably, Flow cytometry can be used in this analysis. Most preferably, FACS assays can be used herein. The use of the herein provided antibodies in such assays allows are more convenient or quicker analysis compared to Western Blot or ELISA techniques.

Flow cytometry uses a laser light source to analyse the size, complexity and physical properties of fresh viable cells in suspension after labelling with fluorescent monoclonal antibodies provided herein. One to two thousand cells can be analysed per second. The advantages of flow cytometry include the ability to rapidly and simultaneously analyse multiple cell parameters. It is recommended that a smear of the specimen should be stained and reviewed microscopically in correlation with flow cytometry to ensure analysis of the correct cell population, to assess cell viability and to guide the selection of antibodies to be used. Flow cytometric analysis may be severely compromised if the samples contain insufficient material or too many dead cells.

Although the acquisition of data can be automated, the interpretation of the results and their clinical significance requires substantial input and critical judgement from trained hematologists or pathologists. Results should be analysed in conjunction with the clinical presentation, cellular morphology and cytogenetics when appropriate.

Flow cytometry/FACS can be used to assess abnormal cell populations. Generally this analysis is requested by hematologists or pathologists to further investigate aberrant cell populations found during microscopy of blood, marrow, lymph nodes or other tissues. FACS can be used to monitor for minimal residual disease. Flow cytometry is one of several methods used to detect minimal residual disease in patients with no clinical or morphological evidence of disease. In patients with a known malignancy, flow cytometry may be useful to detect low levels of persistent disease following therapy. Flow cytometry can be used to quantify cell populations. The use of highthroughput flow cytometry is, for example, disclosed in Gedye (Plos One August 2014|Volume 9|Issue 8|e105602). Such methods can be used to assess/analyze multiple populations within complex samples simultaneously e.g. by co-staining of cells with lineage-specific antibodies, allowing unprecedented depth of analysis of heterogeneous cell populations. Flow cytometry/FACS (and in particular highthroughput Flow cytometry/FACS) combines the advantages of a high-throughput screen with a detection method that is sensitive, quantitative, highly reproducible, and allows in-depth analysis of heterogeneous samples.

A key technique in molecular biology is the electrophoretic separation of molecules, like e.g. proteins, nucleic acids, lipids or carbohydrates with the help of carrier matrices like agarose or polyacrylamide. The most frequently adopted method for the separation of proteins is the so called SDS polyacrylamide gel electrophoresis (SDS-PAGE), by which proteins are separated depending on/according to their molecular weight. To determine or at least estimate the molecular weight of a given protein, it is necessary to compare the migration distance of the protein of unknown molecular weight with the migration distance of proteins of known molecular weights. These proteins are so called protein molecular weight markers or standards and are electrophoretically separated together with the proteins to be analysed. A non-stained protein size marker ladder is, e.g., described in U.S. Pat. No. 5,449,758. Moreover, in DE 102 44 502 B4 molecular weight markers and methods for producing such markers are described while it is mentioned that said protein markers can be transferred onto a membrane and be detected by antibodies against the protein marker. To be able to monitor the migration of the molecular weight markers during electrophoresis, these proteins are commonly covalently coupled to the blue dye Remazol Brilliant Blue R or the vinyl sulfone derivative of Remazol Brilliant Blue R, i.e., Uniblue A (Sigma). These dyes are recognized by the human eye as colour (or as black or as white) upon illumination with visible light which ranges from approximately 380 to 800 nm. Less often, these proteins are commonly covalently coupled to other different-coloured Remazol derivatives like e.g. Remazol Turquoise, Brilliant Red F3B, Brilliant Orange 3R, or Golden Yellow RNL. As an example, a protein marker and a ladder that contains a series of different markers is described in WO 2006/138366 A2 wherein the described protein marker is a product of a protein covalently bound to dye(s). Antibodies or antisera, which are specifically directed against a particular protein, are used to analyze this protein in a protein mixture (e.g. a whole cell lysate), which has been electrophoretically separated. For this purpose, the SDS-PAGE separated proteins are electro-transferred to a carrier membrane (e.g. nitrocellulose or polyvinylidene fluoride [PVDF]), where they can be detected with a specific antibody. This technique is called Western blot or immunoblotting. Immunoblotting is not always required if an in-gel Western blot is carried out. A particular protein is made visible by incubation of the membrane with a primary antibody (in most cases a mouse, rat, goat or a rabbit antibody), which in turn is detected by a secondary antibody, which is directed against mouse, rat, goat or rabbit antibodies and which is coupled to the enzyme horseradish peroxidase (HRP) (or, alternatively, to a fluorescence dye). This enzyme catalyzes the oxidation of luminol leading to the emission of light (chemoluminescence), which then can be detected on X-ray films or with the help of CCD camera-based systems. However, the blue prestained molecular weight markers do not emit any light and are therefore not displayed on the X-ray films. To determine/estimate the molecular weight of the protein recognized by the antibody, it is necessary afterwards (after the emitted light has been detected on the X-ray film) to manually mark the marker protein bands on the X-ray film. This is done by placing the film on the membrane and requires the perfect positioning of the two components. This carries the difficulty that the contours of the membrane are mostly not apparent on the film and thus reference points are lacking. Another source of error is the experimenter and his/her accuracy in mapping the shape of the molecular weight markers on the film. Recently, the company Abcam has put on the market a so called luminol pen (Optiblot Luminol Membrane Pen), with which the marker protein bands can be manually marked on the membrane and subsequently be detected on an X-ray film. The disadvantage of this is again the fact that it requires to manually mark the molecular weight markers, which—as described above—is one of the most common sources of error. Thermo Fisher Scientific on the other hand offers molecular weight markers (Thermo Scientific PageRuler Prestained NIR Protein Ladder), which are marked with a blue dye as well as a fluorescence dye and which can therefore be directly detected by a Western blot analysis. To do so, however, one needs a scanner (e.g. LiCOR, Odyssey, or GE Healthcare Life Sciences, Typhoon).

Herein provided are compositions, in particular pharmaceutical compositions, comprising an antibody/binding molecule as disclosed herein or having essentially the same biological activity (like binding to the same epitope) of an antibody/binding molecule obtained or obtainable from hybridoma 8B2-H6-10.7 deposited under accession number DSM ACC3249 with the depositary institute DSMZ on Sep. 12, 2014. These pharmaceutical compositions can optionally further comprise one or more pharmaceutically acceptable excipient(s). These pharmaceutical compositions can be used in medicine or as a medicament. Preferably, the pharmaceutical compositions are for use in the treatment of a myeloid malignancy.

In a certain aspect, the present invention relates to a composition comprising the antibody/binding molecule directed against/specifically binding to a mutant calreticulin protein as defined herein or as produced by the above described process, a nucleic acid molecule as described herein, a vector as described herein, a host as described herein and/or the deposited hybridoma as disclosed herein. Preferably, the composition comprises the antibody/binding molecule as defined and provided herein. The composition may further comprise (a) secondary antibody/antibodies that is/are specifically binding to the primary antibody (i.e. the antibody specifically binding to a mutant calreticulin protein) as defined and provided in the present invention. The secondary antibody/antibodies can be conjugated to a therapeutic agent as defined above (in particular an anticancer/cytotoxic agent or a toxin) or a diagnostic agent as defined and explained herein above. The primary antibody is preferably an IgG antibody, such as a human or murine IgG antibody. The secondary antibody may be a goat anti-human IgG secondary antibody. The secondary antibody may also be any of the antibody types as described herein above in context of the anti-mutant calreticulin protein antibodies provided herein.

The herein above described composition can be a pharmaceutical composition, optionally further comprising one or more pharmaceutically acceptable excipient(s) like, inter alia, stabilizers or carriers. Corresponding excipients are also provided herein below as non-limiting examples. In accordance with this aspect, the antibody as provided herein, or the antibody as produced by the herein above described process, the nucleic acid molecule described herein, the vector described herein, the host as described herein and/or the composition (in particular the pharmaceutical composition) can be for use in medicine. Preferably, the antibody as provided herein (optionally contained in the composition as defined above) is for use in medicine. In one aspect, the antibody is conjugated to a therapeutic agent.

In a certain aspect, the present invention relates to the use of the antibody as defined or provided herein, the antibody as produced by the herein described process, the nucleic acid molecule as described herein, the vector as described herein, the host and/or the hybridoma as described herein for the preparation of a pharmaceutical composition for the treatment of a myeloid malignancy. Preferably, the present invention relates to the use of the antibody as defined or provided herein for the preparation of a pharmaceutical composition for the treatment of a myeloid malignancy.

It is shown herein that mutant calreticulin is present on the cell surface or extracellular side of the plasma membrane. Therefore, it provides a therapeutic target for the herein provided antibodies. The following non-limiting therapeutic applications are envisaged:

The antibody can be conjugated to cytotoxic agents and the antibody can be internalized by the cells leading to cell death. The antibody can be used to generate an immune response against the mutant CALR protein, so that the endogenous immune system would recognize it as ‘non-self’. The mutant CALR expressing cells can then be killed by the complement system and/or by antibody dependent cellular cytotoxicity (ADCC).

In a certain aspect, the present invention relates to the antibody as defined and provided herein, the antibody as produced by the herein described process, the nucleic acid molecule as described herein, the vector as described herein, the host, the hybridoma and/or the composition as described herein for use in the treatment a myeloid malignancy.

In a certain aspect, the present invention relates a method for the treatment of a myeloid malignancy, said method comprising the administration of the antibody/binding molecule as defined and provided herein, the antibody as produced by the herein described process, the nucleic acid molecule as described herein, the vector as described herein, the host, the hybridoma and/or the composition as described herein to a subject in need of such a treatment. A “patient” or “subject” for the purposes of the present invention includes both humans and other animals, particularly mammals, and other organisms. Thus, the methods are applicable to both human therapy and veterinary applications. In the preferred embodiment the subject is a mammal, and in the most preferred embodiment the subject is a human.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an insufficient immune response from occurring in a subject which may be predisposed to the disease; (b) inhibiting the disease, i.e. arresting its development; or (c) relieving the disease, i.e. causing regression of the disease.

“Treating” or “treatment” does not necessarily require a complete cure. It means that the symptoms of the underlying disease are at least reduced, and/or that one or more of the underlying cellular, physiological, or biochemical causes or mechanisms causing the symptoms are reduced and/or eliminated. It is understood that reduced, as used in this context, means relative to the state of the disease, including the molecular state of the disease, not just the physiological state of the disease.

In one aspect, the treatment of the myeloid malignancy comprises administering to the subject or patient a therapeutically effective amount of the herein disclosed and provided antibody that specifically binds to a mutant calreticulin protein (or a fragment of the antibody etc.). In one aspect, the antibody that specifically binds to a mutant calreticulin protein can reduce expression levels of mutant calreticulin. In one aspect, the antibody that specifically binds to a mutant calreticulin protein can reduce levels of activity of mutant calreticulin protein. In one aspect, the antibody that specifically binds to a mutant calreticulin protein inhibits or reduces proliferation; causes cytotoxicity; inhibits or reduces metastasis; modulates, inhibits or reduces cell adhesion; modulates, inhibits or reduces migration; or modulates, inhibits or reduces invasion of myeloid malignancy cells expressing mutant calreticulin protein. In one aspect, the antibody that specifically binds to a mutant calreticulin protein inhibits or reduces proliferation of myeloid malignancy cells expressing mutant calreticulin protein. In one aspect, the antibody antibody that specifically binds to a mutant calreticulin protein causes cytotoxicity to myeloid malignancy cells expressing mutant calreticulin protein. In one aspect, the antibody that specifically binds to a mutant calreticulin protein reduces or inhibits migration of myeloid malignancy cells expressing mutant calreticulin protein.

Confirming the anti-myeloid malignancy properties of the herein provided anti-mutant calreticulin protein antibodies can be done using standard assays. For example, a myeloid malignancy cell line is grown and propagated in culture according to methods well known to one of ordinary skill in the art. Various dosages of potentially therapeutic antibodies or fragments thereof or conjugates thereof according to the invention are applied to various cultures of the cell line. The treated cultures and control cultures (treated with a sham antibody or fragment) are then followed over time and scored for reduction in proliferation; reduction in cellular growth; reduction in colony formation; appearance of cytotoxicity; reduction in cell-adhesion; reduction of cell invasion; reduction of degradation of the extracellular matrix; or reduction in cell migration or reduction in cell action through different extracellular matrix proteins. In vivo, the antibodies/binding molecules of the invention or conjugates thereof can be tested in animal models of myeloid malignancy. Routes of antibody administration into animal models like mice, rats etc. include intravenous or intraperitoneal administration. Various dosages of potentially therapeutic antibodies or fragments thereof according to the invention (or combinations of a mix of antibodies or combination of the antibodies with chemotherapy) can be tested in in vivo models. The treated animals and control animals (treated with a sham antibody or fragment or no antibody at all) are then followed over time and scored for reduction pathological symptoms, like appearance of cytotoxicity; reduction in tumor cell-adhesion; reduction in tumor cell migration or increase in survival.

In one aspect, the antibody that specifically binds to mutant calreticulin protein induces, enhances, or mediates ADCC (antibody dependent cellular cytotoxicity) against cells to which it binds. ADCC is one of the mechanism by which an antibody can have a therapeutic effect. ADCC is a cell mechanism where an effector cell of the immune system, mainly Natural Killer cells (NK), lyses a target cell which has been previously bound by specific antibodies. NK cells have specific receptors such as FcγRIIIa which recognize the Fc fragment of immunoglobulins and are responsible for the ADCC response. To test if the antibodies of the invention have a therapeutic effect through a ADCC mechanism, an in vitro assay can be performed in which target cells will be incubated with different antibodies and natural killer cells from human or mouse origin. The effect of the antibodies on the cells can be measured by the occurred lyses.

In one aspect, the antibody that specifically binds to mutant calreticulin protein induces, enhances, or mediates CDC (complement dependent cytotoxicity) against cells to which it binds. CDC is another immune mechanism to exert cytotoxicity on tumor cells. CDC is a cytolytic cascade mediated by complement proteins in the serum. CDC is initiated by the binding of C1q to the constant region of cell bound antibody molecule.

The antibody that specifically binds to mutant calreticulin protein can be conjugated to another molecule. In a more specific aspect, the antibody is conjugated to a therapeutic agent, such as a toxin, a radioactive agent, inhibitory peptide, or an anti-tumor drug as described herein. The antibody (or fragment thereof) of this aspect can be provided as a pharmaceutical composition comprising the antibody (or fragment thereof) conjugated to the therapeutic agent and a pharmaceutically acceptable excipient.

Pharmaceutical compositions of this invention also can be administered in combination therapy (“cotherapy”), i.e., combined with other agents. For example, the combination therapy can include an antibody specifically binding to a mutant calreticulin protein of the present invention combined with at least one other therapeutic agent (e.g. anti-myeloid malignancy agent) or other therapeutic intervention. If the at least one other therapeutic agent is used in such a “cotherapy” the therapeutic agent is not conjugated (as defined above) to the herein provided antibody. It is envisaged that the antibody used in cotherapy with one or more other therapeutic agents may, in itself, be conjugated to one or more of the therapeutic agents as defined herein above.

The administration of the other therapeutic agent can be prior to, concurrent to or after the administration of the antibody of the invention. The antibody of the invention and the one or more other therapeutic agents may also be combined into a single dosage unit. Furthermore, the invention includes a pharmaceutical composition comprising two or more antibodies to mutant calreticulin protein. Examples of therapeutic agents that can be used in combination therapy are described in greater detail below.

In one aspect, the therapy can comprise identifying a patient having a risk factor for myeloid malignancy or being suspected of suffering from a myeloid malignancy. In one aspect, the risk factor for a myeloid malignancy can be age, ethnicity, family history of myeloid malignancy, or a genetic predisposing gene or variant thereof. Risk factors for a myeloid malignancy are known to the skilled artisan. Mutant calreticulin protein itself can be a risk factor. For example, the presence of mutant calreticulin protein (or a fragment thereof) (or corresponding nucleic acid encoding same or a part thereof) in a sample of a patient being suspected of suffering from a myeloid malignancy or having a risk factor for myeloid malignancy (like age, ethnicity, family history of myeloid malignancy, or a genetic predisposing gene or variant thereof) can be determined. A patient with a detectable level of mutant calreticulin protein can be treated with the herein provided antibody/antibodies.

In one embodiment of the invention the subject or patient to be treated was previously treated or is currently being treated with radiation therapy. In a more specific embodiment, the invention provides a method of treatment of a myeloid malignancy in a patient wherein said patient was previously treated or is currently being treated with radiation therapy. In one aspect of this embodiment, the treatment comprises identifying a patient previously treated or is currently being treated with radiation therapy and administering to said patient a therapeutic antibody as defined herein. Radiation therapy for a myeloid malignancy is generally classified as external or internal. External radiation therapy usually involves the focusing of high energy beams of energy (e.g., x-rays) on the affected area. Internal radiation therapy involves implanting a radioactive substance or composition comprising a radioactive substance near or inside the myeloid malgi (also referred to as brachytherapy, internal radiation therapy, and/or radiation brachytherapy).

In a certain aspect, the subject or patient will be treated or is currently being treated with a chemotherapy or a radiotherapy.

A patient suffering from a myeloid malignancy can be treated in accordance with the present invention, wherein said patient had discontinued a prior treatment due to disease progression. In one aspect, disease progression occurred due to the developed chemoresistance to the prior treatment. In one aspect, said chemoresistance was or is correlated to (increased) expression or activation of mutant calreticulin. In a specific aspect the antibodies to mutant calreticulin protein confer chemosensitivity to chemoresistant cells, or increase chemosensitivity of the cells.

The ability of an antibody of the invention to confer or increase chemosensitivity to chemoresistant cells can be tested as follows. Chemoresistant target cells (e.g, expressing mutant calreticulin or overexpressing mutant calreticulin) are plated on 96 well plates and incubated with the herein provided antibodies to be tested with and without a chemotherapeutic agent under conditions sufficient for cell growth and proliferation. The effect of the treatments on cell proliferation will be measured by an Alamar Blue assay or similar assays as described herein e.g., cytotoxicity.

As mentioned previously, in a certain aspect the invention relates to a pharmaceutical composition comprising, inter alia, an antibody or binding molecule of the invention, as described herein, optionally further comprising one or more pharmaceutically acceptable excipient(s).

As used herein, “pharmaceutically acceptable excipient” relates to any component of a pharmaceutical composition other than the active ingredient and includes any and all carriers, solvents, dispersion media, coatings, antibacterial and antifungal agents, isotonic and absorption delaying agents, and the like that are physiologically compatible. Preferably, the excipient is suitable for intravenous, intramuscular, subcutaneous, parenteral, spinal or epidermal administration (e.g., by injection or infusion). Depending on the route of administration, the active compound, i.e., antibody, may be coated in a material to protect the compound from the action of acids and other natural conditions that may inactivate the compound. The pharmaceutical compounds of this invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N.N′-dibenzylethylenediamine, N-methylglucamine, chloroprocaine, choline, diethanolamine, ethylenediamine, procaine and the like.

A pharmaceutical composition of this disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA)₅ butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of this disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient (i.e. the herein provided antibody, nucleic acid molecules etc.) which can be combined with a excipient to produce a single dosage form will vary depending upon the subject being treated, and the particular mode of administration. The amount of active ingredient which can be combined with a excipient to produce a single dosage form will generally be that amount of the composition which produces a therapeutic effect. Generally, this amount will range from about 0.01 percent to about ninety-nine percent of active ingredient, preferably from about 0.1 percent to about 70 percent, most preferably from about 1 percent to about 30 percent of active ingredient in combination with (a) pharmaceutically acceptable excipient(s).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical excipient. The specification for the dosage unit forms of this disclosure are dictated by and directly dependent on (a) the unique characteristics of the active compound and the particular therapeutic effect to be achieved, and (b) the limitations inherent in the art of compounding such an active compound for the treatment of sensitivity in individuals. For administration of the antibody, the dosage typically ranges from about 0.0001 to 100 mg/kg, and more usually 0.01 to 10 mg/kg, of the host body weight. Typically, when the antibody is administered as an ADC, the ADC will be administered at a dose of less than 1 mg/kg.

Antibody/binding molecules etc. can also be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage”, “therapeutically effective amount” or “effective amount” of an anti-mutant calreticulin antibody of this invention preferably results in a decrease in severity of disease symptoms, an increase in frequency and duration of disease symptom-free periods, or a prevention of impairment or disability due to the disease affliction.

A composition of the present disclosure can be administered via one or more routes of administration using one or more of a variety of methods known in the art. As will be appreciated by the skilled artisan, the route and/or mode of administration will vary depending upon the desired results. Preferred routes of administration for antibodies of this disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of this disclosure can be administered via a non-parenteral route, such as a topical, epidermal or mucosal route of administration, for example, intranasally, orally, vaginally, rectally, sublingually or topically.

The active compounds can be prepared with excipients that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, eds, Marcel Dekker, Inc., New York, 1978. Therapeutic compositions can be administered with medical devices known in the art. For example, in a preferred embodiment, a therapeutic composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art. In certain embodiments, therapeutic antibodies of this disclosure can be formulated to ensure proper distribution in vivo. For example, the blood-brain barrier (BBB) excludes many highly hydrophilic compounds. To ensure that the therapeutic compounds of this disclosure cross the BBB (if desired), they can be formulated, for example, in liposomes. For methods of manufacturing liposomes, see, e.g., U.S. Pat. Nos. 4,522,81 1; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); marmosides (Umezawa et al. (1988) Biochem. Biophys. Res. Commun. 153:1038); antibodies (P. G. Bloeman et al. (1995) FEBS Lett. 357: 140; M. Owais et al. (1995) Antimicrob. Agents Chemother. 39: 180); surfactant protein A receptor (Briscoe et al. (1995) Am. J. Physiol. 1233:134); pi 20 (Schreier et al. (1994) J. Biol. Chem. 269:9090); see also K. Keinanen; M. L. Laukkanen (1994) FEBS Lett. 346: 123; JJ. Killion; IJ. Fidler (1994) Immunomethods 4:273.

When used in the therapy of myeloid malignancies, examples of chemotherapeutic agents that may be used in combination with the antibodies of the invention include, but are not limited to, antimetabolites (e.g., methotrexate, azathioprine, 6-mercaptopurine, 6-thioguanine, cytarabine, 5-fluorouracil, decarbazine, capecitabine), alkylating agents (e.g., mechlorethamine, thiotepa, chlorambucil, melphalan, carmustine (BCNU), lomustine (CCNU), cyclophosphamide, ifosfamide, busulfan, dibromomannitol, streptozotocin, mitomycin C, cis-dichlorodiamine platinum (II) (DDP), cisplatin, carboplatin, oxaliplatin, nedaplatin, satraplatin, triplatin tetranitrate, procarbazine, altretamine and tetrazines), anthracyclines (e.g., daunorubicin, doxorubicin, valrubicin, idarubicin, epirubicin, and mitoxantrone), antibiotics (e.g., dactinomycin, bleomycin, mithramycin, and anthramycin (AMC)), topoisomerase inhibitors (e.g. irinotecan, topotecan and camptothecin), anti-mitotic agents (e.g., vinca alkaloids such as vincristine and vinblastine, taxanes such as paclitaxel (also known as taxol), cabazitaxel and docetaxel, and other tubulin polimeryzation inhibitors such as monomethyl auristatin E (MMAE), maytansine derivatives like mertansine (also known as DM1) and DM4), and protein kinase inhibitors such as imatinib (gleevec), nilotinib and dasatinib.

For other co-therapeutic approaches for example for the use of the inventive antibodies/binding molecules in anti-inflammatory therapy, the following drugs/agents may be employed: steroids such as Glucocorticoids, Non-Steroidal anti-inflammatory drugs such as aspirin. ibuprofen, naproxen or Immune Selective Anti-Inflammatory Derivatives (ImSAIDs) such as the peptide phenylalanine-glutamine-glycine (FEG). For the treatment of atherosclerosis the antibodies of the invention can be combined with e.g. statins or niacin.

The following relates to antibody dependent and complement dependent cytotoxicity. In one embodiment, the invention relates to an antibody specifically binding to mutant calreticulin protein that induces, enhances, or mediates antibody-dependent cellular cytotoxicity (ADCC). ADCC as described above is a type of immune reaction in which a target cell is coated with antibodies and killed by certain types of white blood cells, particularly NK cells. The white blood cells bind to the antibodies and release substances that kill the target cells or microbes. Not all antibodies produce ADCC. Thus, in one aspect, the invention relates to an antibody specifically binding to mutant calreticulin protein that can induce, enhance or mediate ADCC. Furthermore, antibodies of the invention specifically binding to mutant calreticulin protein can be engineered to have improved, increased or enhanced ADCC. For example an antibody of the invention that does not induce, enhance, or mediate ADCC can be engineered, e.g., by making certain amino acid modifications to the antibody or by producing the antibody in certain strains of cells, to induce, enhance or mediate ADCC or have improved/enhanced ADCC properties.

In one aspect, an antibody specifically binding to mutant calreticulin protein has antibody-dependent cellular cytotoxicity when used in a human subject. One example of an antibody with increased or improved ADCC activity is an antibody specifically binding to mutant calreticulin protein that is defucosylated. The antibody specifically binding to mutant calreticulin protein and having ADCC or increased ADCC can be generated by producing the antibody in a cell-line that lacks or has decreased alpha-1,6-fucosyltransferase activity. The antibody specifically binding to mutant calreticulin protein and having ADCC or increased ADCC can be generated by producing the antibody in a cell-line that has reduced or lacks GDP-fucose transporter activity. The antibody specifically binding to mutant calreticulin protein having ADCC or increased ADCC can be generated by producing the antibody in a cell-line that has reduced or lacks GDP-mannose 4,6-dehydratase activity. The antibody specifically binding to mutant calreticulin protein and having ADCC or increased ADCC is generated by producing the antibody in a cell-line that has reduced or lacks both alpha-1,6-fucosyltransferase activity and GDP-mannose 4,6-dehydratase activity; see e.g., Yamane-Ohnuki et al. (2004) Biotechnol Bioeng. 87(5):614-22; Imai-Nishiya et al. (2007) BMC Biotechnology 7:84. ADCC can be enhanced or improved by increasing the levels of interleukin-21 (IL-21) in a patient or by treating the patient with IL-21 in combination with the antibody of the invention. See e.g., Watanabe et al. Br J Cancer. 2010, 102(3), 520-9.

The antibody specifically binding to mutant calreticulin protein can enhance, induce or mediate complement dependent cytotoxicty (CDC). Antibodies of the invention can be engineered to have improved, increased or enhanced CDC. For example, an antibody of the invention that does not induce or mediate CDC can be engineered, e.g., by making certain modifications to the antibody like amino acid mutations in Fc or the hinge region thereby improving or enhancing CDC. Another method of producing CDC or enhancing an antibody's CDC is by shuffling IgG1 and IgG3 sequences within the heavy chain constant region. See e.g., Natsume et al. (2008) Cancer Res. 68:3863-3872.

The following relates to conventional therapy of exemplary myeloid malignancies. These therapies can be used e.g. after positive diagnosis of the herein provided anti-mutant calreticulin antibodies or in combination therapy with the herein provided anti-mutant calreticulin antibodies. The therapeutic compounds mentioned below may, for example, also be conjugated to the herein provided antibodies for the herein disclosed therapeutic applications of the antibody, like treatment of a myeloid malignancy.

The purpose of treatment for polycythemia vera is to reduce the number of extra blood cells. Treatment of polycythemia vera may include, phlebotomy, chemotherapy with or without phlebotomy, biologic therapy using interferon alfa or pegylated interferon alpha and low-dose aspirin.

The treatment of primary myelofibrosis in patients without signs or symptoms is usually watchful waiting. Patients with primary myelofibrosis may have signs or symptoms of anemia. Anemia is usually treated with transfusion of red blood cells to relieve symptoms and improve quality of life. In addition, anemia may be treated with erythropoietic growth factors, prednisone, danazol, thalidomide, lenalidomide, or pomalidomide. Treatment of primary myelofibrosis in patients with other signs or symptoms may include targeted therapy with ruxolitinib (a JAK1 and JAK2 inhibitor), chemotherapy, donor stem cell transplant, thalidomide, lenalidomide, or pomalidomide, splenectomy, radiation therapy to the spleen, lymph nodes, or other areas outside the bone marrow where blood cells are forming, biologic therapy using interferon alfa or erythropoietic growth factors, or the inclusion in a clinical trial of other targeted therapy drugs.

Treatment of essential thrombocythemia in patients younger than 60 years who have no signs or symptoms and an acceptable platelet count is usually watchful waiting. In some cases, the patient can take aspirin to help prevent blood clots. Treatment of other patients may include Chemotherapy, hydroxyurea, Anagrelide therapy, biologic therapy using interferon alfa or pegylated interferon alpha, platelet apheresis.

The JAK-binding inhibitor ruxolitinib shows promise for curative and supportive treatment. Ruxolitinib has been approved by the Food and Drug Administration) for use in the treatment of high and intermediate risk myelofibrosis in 2011; see Tefferi Mar. 22, 2012; Blood: 119 (12) Also Ostojic reports that ruxolitinib is used in the therapy of myelofibrosis; see Ostojic Therapeutics and Clinical Risk Management 2012:8 95-103.

JAK inhibitors that are currently used in clinical trials for myeloproliferative neoplasms include, besides ruxolitinib, SAR302503, CYT387, lestaurtinib, SB1518, AZD1480, BMS911543, LY2784544, NS-018, and XL019; see Tefferi Mar. 22, 2012; Blood: 119 (12).

An exemplary formula of ruxolitinib ((3R)-3-cyclopentyl-3-[4-(7H-pyrrolo[2,3-d]pyrimidin-4-yl)pyrazol-1-yl]propanenitrile; trade name Jakafi, Jakavi) is shown below:

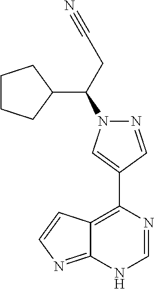 [SMILES: N#CC[C@H](C1CCCC1)n1cc(-c2nc[nH]c3nccc2-3)cn1]

Refractory anemia with ringed sideroblast and thrombocytosis may require blood transfusions and other supportive therapy to remedy anemia, including high doses of pyrodoxine (Vitamin B6). Bone marrow transplant is also an option. RARS-T may also progress to leukemia.

The use of above therapies is contemplated for patients diagnosed positive or negative for the presence of mutant calreticulin in accordance with the present invention, either alone or in combination with therapies (e.g. antibodies) specifically targeting the mutant calreticulin. Accordingly, therapies (e.g. antibodies) that target mutant CALR, can likewise be useful in treatment if used as monotherapy or in combination with other therapies. For example, interferon alfa therapy can be used to treat patients with MPN (like essential thrombocythemia patients) diagnosed positive for the presence of mutant calreticulin in accordance with the present invention.

If, for example, the patient is tested positive for the presence of mutant calreticulin and (a) JAK2 mutation(s), the use of JAK inhibitor(s) (like ruxolitinib) is contemplated herein. Depending on clinical parameters, (e.g age, prognosis of the patient) also further therapies, like stem cell transplantation can be used to treat e.g. a patient tested positive for the presence of mutant calreticulin.

As used herein, the terms “comprising”/“including”/“having” or grammatical variants thereof are to be taken as specifying the stated features, integers, steps or components but do not preclude the addition of one or more additional features, integers, steps, components or groups thereof. This term encompasses the terms “consisting of” and “consisting essentially of” Thus, the terms “comprising”/“including”/“having” mean that any further component (or likewise features, integers, steps and the like) can be present. The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed antibody, composition or method.

Thus, the term “consisting essentially of” means that specific further components (or likewise features, integers, steps and the like) can be present, namely those not materially affecting the essential characteristics of the antibody, composition or method. In other words, the term “consisting essentially of” (which can be interchangeably used herein with the term “comprising substantially”), allows the presence of other components in the antibody, composition or method in addition to the mandatory components (or likewise features, integers, steps and the like), provided that the essential characteristics of the antibody, composition or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

As used herein the term “about” refers to ±10%.

The present invention is further described by reference to the following non-limiting figures and examples.

Unless otherwise indicated, established methods of recombinant gene technology were used as described, for example, in Sambrook, Russell “Molecular Cloning, A Laboratory Manual”, Cold Spring Harbor Laboratory, N.Y. (2001)) which is incorporated herein by reference in its entirety.

The Figures show:

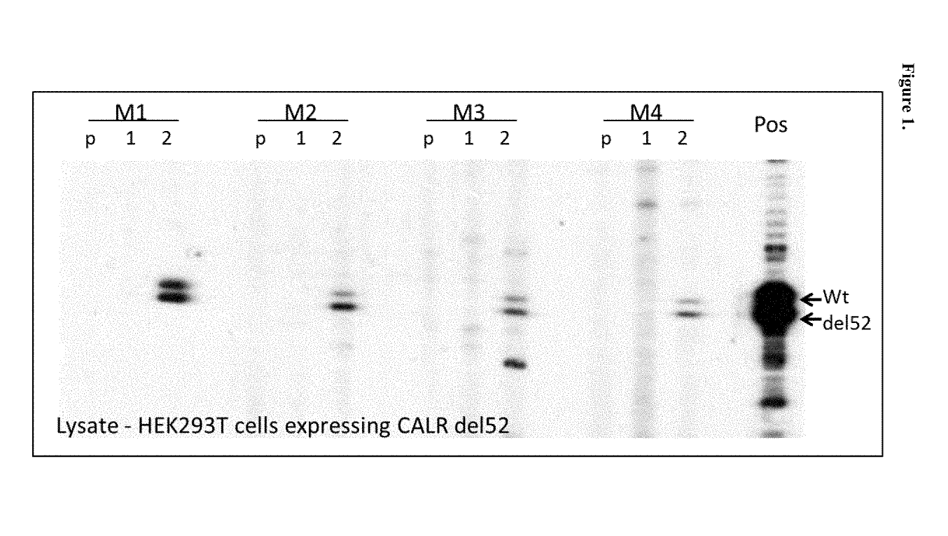

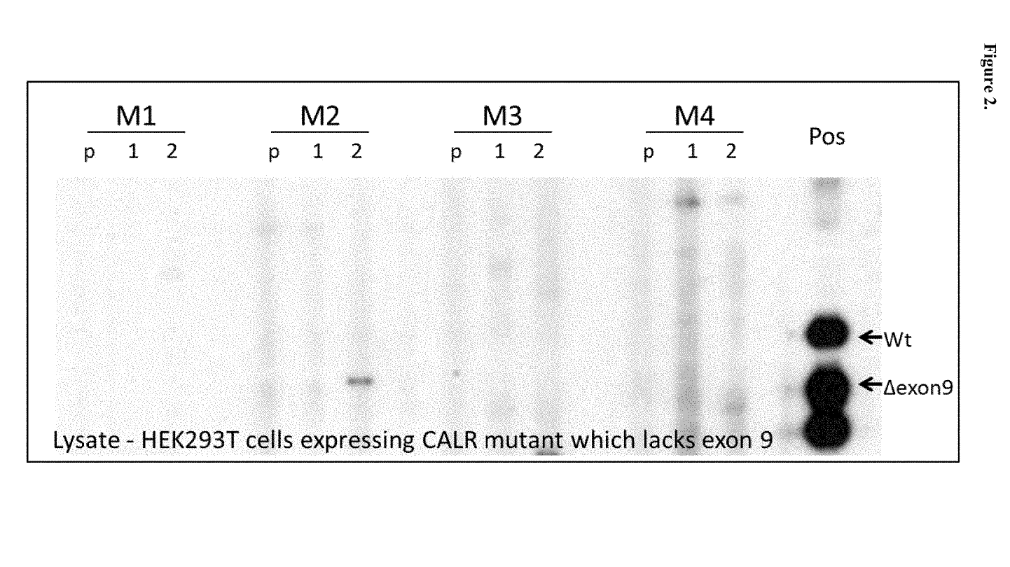

FIG. 1.

Western blot analysis was performed probing the sera of the four immunized mice, against lysates from HEK293T cells expressing the CALR mutant del52. The figure shows that the sera from all four immunized mice did not have any antibodies against the mutant calreticulin peptide before immunization—p (pre-immunized lanes). They generated specific antibody after 2 booster doses.

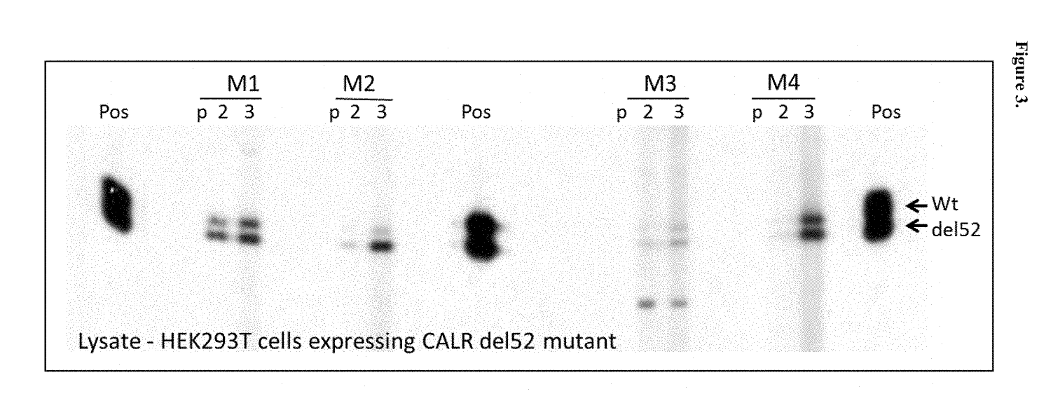

FIG. 2.

Western blot analysis was performed probing the sera of the four immunized mice, against lysates from HEK293T cells expressing the CALR Δexon9. The figure shows that the sera from all four immunized mice did not have any antibodies against the CALR Δexon9 in both the pre-immunized sera (p) or after booster doses.

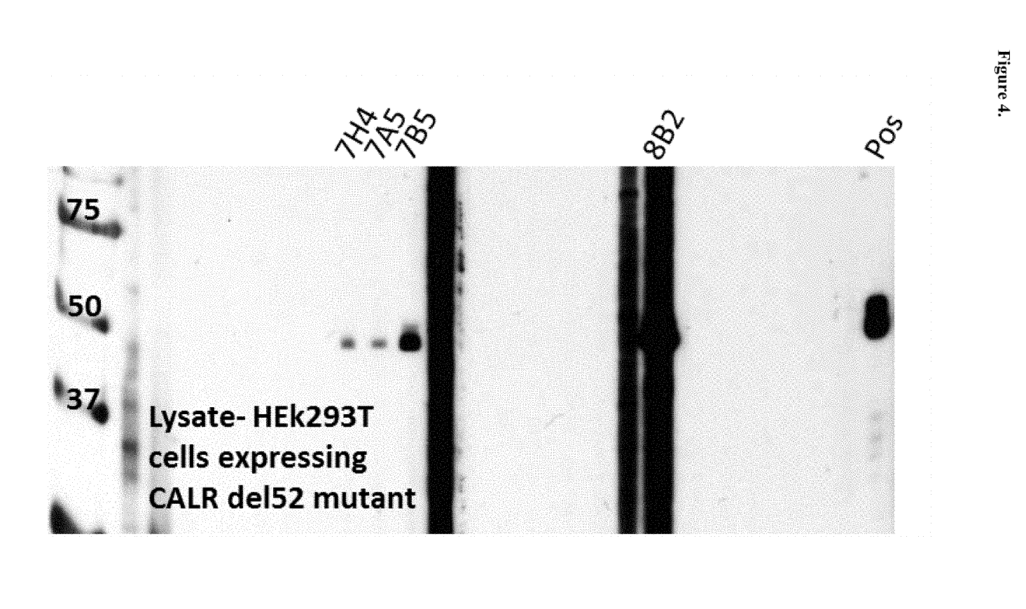

FIG. 3.

Western blot analysis was performed probing the sera of the four immunized mice, against lysates from HEK293T cells expressing the CALR mutant del52. The figure shows that the sera of all four immunized mice had more specific antibody against calreticulin mutant after the third booster dose.

FIG. 4.

Western blot analysis was performed probing the supernatant of hybridoma colonies against lysates from HEK293T cells expressing the CALR mutant del52. The figure shows that four clones produced antibody specifically binding the mutant CALR.

FIG. 5.

Western blot analysis was performed probing the supernatant of 8B2-H6 hybridoma clone, against lysates from HEK293T cells expressing the CALR mutant del52. The figure shows that this clone produced antibody specifically binding the mutant CALR and could detect the mutant CALR even at a dilution of 1:27.

FIG. 6.

The figure shows agarose gel image of PCR products obtained by amplification of variable regions of the heavy and light chains of the immunoglobulin(s) produced by the 8B2-H6 clone.

FIG. 7.

Western blot analysis was performed probing the different eluted fractions of antibody purified from 8B2-H6-10.7 clone, against lysates from HEK293T cells expressing the CALR mutant del52. The figure shows that fraction 4 clones contained the highest amount of mutant CALR specific antibody.

FIG. 8.

The antibody from the above mentioned fraction 4 (1 μg/sample) was used to stain Ba/F3-MPL cells over-expressing different CALR constructs. Anti-mouse APC (ebiosciences #17-4010-82) was used as secondary antibody. FACS analysis was performed. The antibody specifically recognizes the mutant CALR present on the surface of the respective cells.

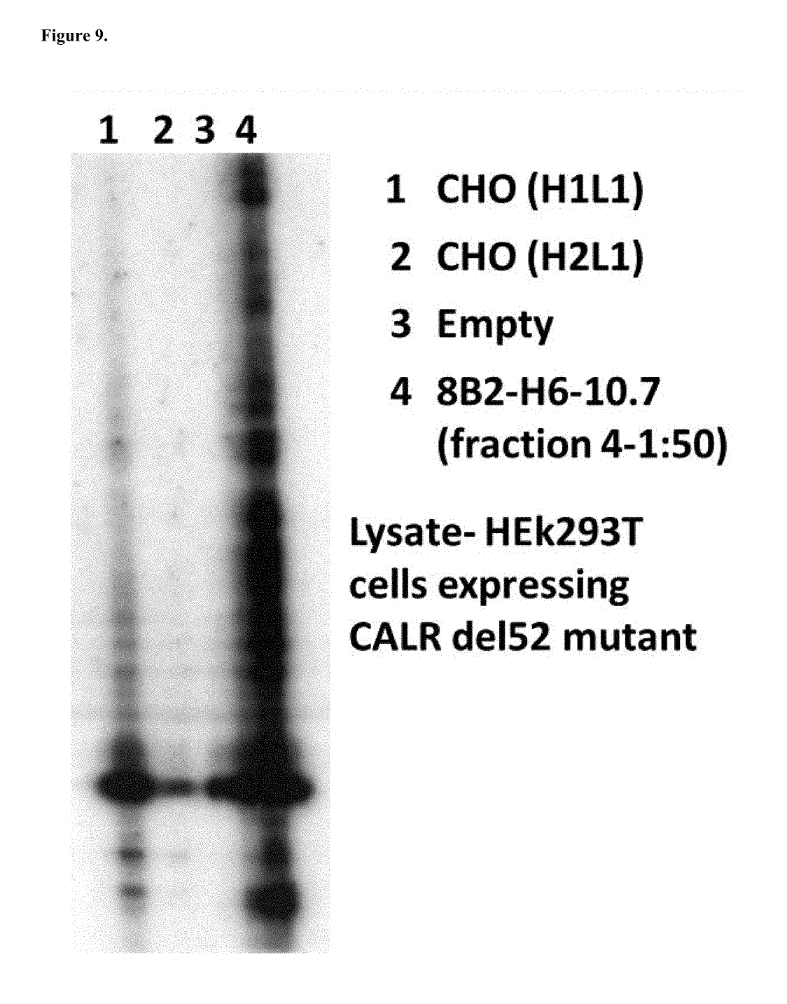

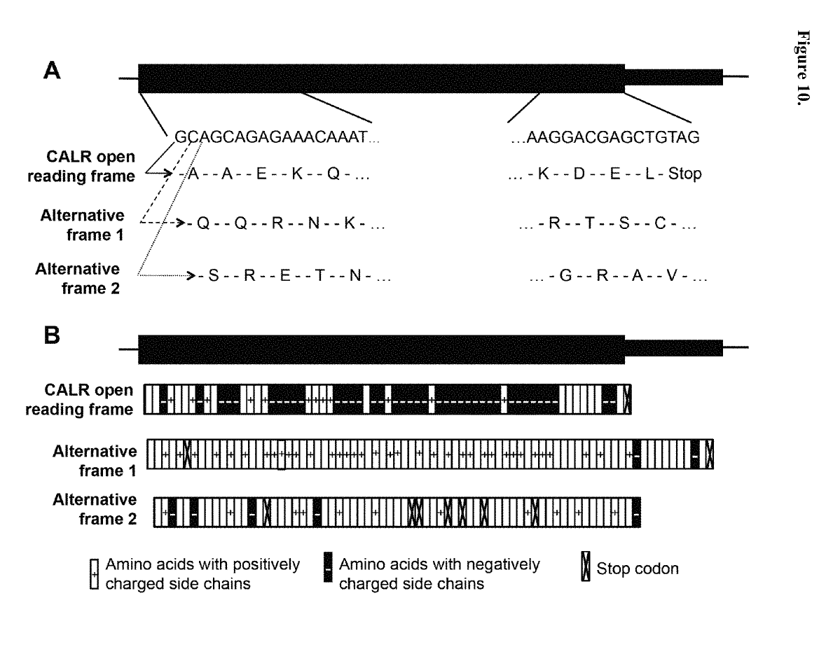

FIG. 9.

Western blot analysis was performed probing the supernatant of CHO cells transfected with H1L1 or H2L1 antibody, against lysates from HEK293T cells expressing the CALR mutant del52. The figure shows that although both the H1L1 and not H2L1 antibodies recognize the mutant CALR, the H2L1 antibody is more specific and does not give a background.

FIG. 10.

Mutational pattern of CALR mutations in MPN patients.

The wide black bar represents exon 9 of CALR, the narrow bar the 3′ UTR of the gene, the thin line intronic and intergenic regions.

A: indicated are the cDNA sequence in the beginning and end of exon 9. Below the cDNA sequence are the amino acid sequences derived from the three alternative reading frames. B: The three reading frames result in different peptide compositions, especially with respect to the charge of amino acids. C: Summary of all mutations detected in MPN patients and their position within CALR exon 9. Bars indicate deletion events, letters inserted sequences. Independent insertions and deletions are depicted above the exon 9 scheme, combined insertion/deletion events below. D: The specific peptide makeup of wild type CALR and of the two most frequently detected types of mutations. B, D: Each box represents an amino acid. Black boxes with ‘-’ sign are negatively charged amino acids, boxes with ‘+’ sign are positively charged amino acids. Crossed boxes represent stop codons. E: Relative frequencies of all 36 mutation types observed in CALR.

The Example illustrates the invention.

### EXAMPLE 1: GENERATION OF CALR MUTANT SPECIFIC ANTIBODIES IN MICE

The CALR mutations associated with MPN occur exclusively in the last exon of the gene (exon 9). These mutations are insertions and/or deletions that result in a ‘frameshift’ mutation to a very specific alternative reading frame, leading to synthesis of a novel C-terminal peptide in the mutant. As all the mutations result in generation of the same alternative reading frame, the C-terminal peptide has the same sequence in all the CALR mutants (Klampfl et al., 2013 (loc. cit.)).

A synthetic peptide with the c-terminal end sequence of the mutant calreticulin protein (Sequence—RRKMSPARPRTSCREACLQGWTEA-), conjugated to the Keyhole Limpet Hemocyanin (KLH) was used to immunize four wild type C57Bl/6 mice.

The mice received 3 booster doses after the primary immunization. The sera of the mice was tested (pre-immune and after boosters) for the presence of mutant calreticulin specific antibodies by western blot analysis of lysates from HEK cells that over-expressed the CALR del52 and the artificially generated CALR mutant that lacks the exon 9 (Δexon9, which lacks the mutant peptide). The lysates were run on 8% polyacrylamide gels and probed with the mouse serum. Anti-mouse antibody conjugated to HRP (GE NA931) was used as secondary antibody. After the second booster, the sera from all four mice had CALR mutant specific antibodies that detected the CALR del52 mutant (FIG. 1), but not the control exon 9 deleted CALR (FIG. 2). FIG. 10 shows the CALR del52 mutation. The exon 9 deleted CALR is a truncated version of wild-type CALR(1-1056 base pairs). Anti-calreticulin antibody (Millipore MABT145) was used as positive control (Pos), which recognizes all three forms of calreticulin—wild type, mutant del 52 and deleted exon 9. The upper band in the Western Blots using the sera from the immunized mice (FIG. 1) represents the unprocessed mutant CALR which has a 17 amino acid leader peptide. The unprocessed mutant CALR with the 17 amino acid leader peptide is not the wild type CALR. Thus, FIG. 1 shows that the antibodies specifically bind to mutant calreticulin protein (or, particularly, the specific, C-terminus of the mutant calreticulin). FIG. 2 confirms that the sera from the immunized mice do not cross-react with the N-terminus of mutant calreticulin. Here it is shown that the sera from the mice do not recognize the deleted exon9 version of CALR.

The signal, from the sera of all four mice, was stronger after the third booster was applied (FIG. 3). The C-terminal peptide of the mutant calreticulin (mentioned above) is immunogenic and can successfully be used to generate specific antibodies, in particular monoclonal antibodies against the mutant calreticulin.

To generate mutant CALR specific monoclonal antibodies, the splenocytes from the mouse M4 were harvested and fused with myeloma cell line to produce hybridoma cells. The hybridomas were screened for production of mutant CALR specific monoclonal antibody by Western blotting, using the supernatant as probe. Four clones, producing mutant CALR specific monoclonal antibody, were identified—7H4, 7A5, 7B5 and 8B2 (FIG. 4). As the 8B2 clone showed the strongest mutant CALR specific band, cells from this clone were plated in serial dilution (one cell per well, in a 96 well plate), to isolate a single cell clone producing the mutant CALR specific monoclonal antibody. The screening was again performed by using the supernatant as probe in Western blotting. The clone 8B2-H6 was identified as the single cell clone producing mutant CALR specific monoclonal antibody (FIG. 5).

The RNA from clone 8B2-H6 was extracted and cDNA was prepared. Primers from the Mouse IgG Library primer set (Progen) were used to amplify the variable regions of the specific immunoglobulin heavy chain and light chain produced by this clone (FIG. 6) and the PCR product was sequenced. The amplification was performed using primers from the Mouse IgG Library primer set (Progen). Specifically, the primer pairs A/B+M generate the H2 heavy chain, C/E/F/G/L+M generate the H1 heavy chain and N/R/T+X generate the L1 light chain.

| | |
|---|---|
| H2 | Forward primer A-GAT GTG AAG CTT CAG GAG TC |
| | Forward primer B-CAG GTG CAG CTG AAG GAG TC |
| | Reverse primer M-GGC CAG TGG ATA GTC AGA TGG GGG TGT CGT TTT |
| | GGC |
| H1 | Forward primer C-CAG GTG CAG CTG AAG CAG TC |
| | Forward primer E-GAG GTG CAG CTG CAA CAA TCT |
| | Forward primer F-GAG GTC CAG CTG CAG CAG TC |
| | Forward primer G-CAG GTC CAA CTG CAG CAG CCT |
| | Forward primer L-GAG GTG CAG CTG GAG GAG TC |
| | Reverse primer M-GGC CAG TGG ATA GTC AGA TGG GGG TGT CGT TTT |
| | GGC |
| L1 | Forward primer N-GAT GTT TTG ATG ACC CAA ACT |
| | Forward primer R-GAC ATT GTG ATG ACC CAG TCT |
| | Forward primer T-GAT ATC CAG ATG ACA CAG ACT |
| | Reverse primer X-GGA TAC AGT TGG TGC AGC ATC |

The amplification was performed on cDNA using the AmpliTaq Gold 360 Master Mix (annealing temperature 55° C.) according to the manufacturer's recommendation.

One light chain sequence (L1) and two unique heavy chain sequences (H1 and H2) were obtained. A ‘blast analysis’ was performed with the nucleic acid sequences obtained against the IMGT database. This database provided the corresponding amino acid sequence in the appropriate reading frame, from the germ line antibody sequences. This database also provided information regarding the framework region and the CDR (complementarity determining region) of the corresponding antibody, for both heavy and light chains. The complementarity determining regions (CDRs) are highlighted in bold letters.

H1:
>DNA
ATATCCTGCAAGGCTTCTGGTTACTCTTTCACTGGTTACTACATACACTG
GGTCAAGCAGAGCCATGGAAAGAGCCTTGAGTGGATTGGATATATTAGTT
GTTACAATGGTGCTTCTAGCTACAACCAGAAGTTCAAGGGCAAGGCCACA
TTTACTGTAGACACATCCTCCAGCACAGCCTACATGCAGTTCAACAGCCT
GACATCTGGAGACTCTGCGGTCTATTACTGTGCAAGTTCTATGGACTACT
GGGGTCAAGGAACCTCAGTCACCGTCTCCTCAGCCAAAACGACACCCCCA
TCTGACTA
>Protein
ISCKASGYSFTGYYIHWVKQSHGKSLEWIGYISCYNGASSYNQKFKGKAT
FTVDTSSSTAYMQFNSLTSGDSAVYYCASSMDYWGQGTSVTVSSAKTTPP
SD
H2:
>DNA
TTGGCCCCAGTAGTCAAAGTAGTACCATTACTACCGTAGTAATAGGGGGG
GTCTCTTGCACAGTAATATGTGGCTGTGTCCTCAGGAGTCACAGAATTCA
ACTGCAGGAAGAACTGGTTCTTGGATGTGTCTCGAGTGATAGAGATTCGA
CTTTTGAGAGATGGGTTGTAGCTAGTGCTACCACTGTAGCTTATGTAGCC
CATCCACTCCAGTTTGTTTCCTGGAAACTGCCGGATCCAGTTCCAGGCAT
AATCACTGGTGATTGAGTAGCCAGTGACAGTGCAGGTGAGGGACAGAGAC
TGAGAATTTTTCACCAGGCCAGGTCCCGACTCCTGAAGCTTTCACATCA
>Protein
KLQESGPGLVKNSQSLSLTCTVTGYSITSDYAWNWIRQFPGNKLEWMGYI
SYSGSTSYNPSLKSRISITRDTSKNQFFLQLNSVTPEDTATYYCARDPPY
YYGSNGTTLTTGA
L1:
>DNA
CAGCCTCCATCTCTTGCAAGTCAAGTCAGAGCCTCTTAGATAGTGATGGA
AAGACATATTTGAATTGGTTGTTACAGAGGCCAGGCCAGTCTCCAAAGCG
CCTAATCTATCTGGTGTCTAAACTGGACTCTGGAGTCCCTGACAGGTTCA
CTGGCAGTGGATCAGGGACAGATTTCACACTGAAAATCAGCAGAGTGGAG
GCTGAGGATTTGGGAGTTTATCATTGCTGGCAAGGTACACATTTTCCGTA
CACGTTCGGAGGGGGGACCAAGCTGGAAATAAAACGGGCTGATGCTGCAC
CAACTGTATCCN
>Protein
ASISCKSSQSLLDSDGKTYLNWLLQRPGQSPKRLIYLVSKLDSGVPDRFT
GSGSGTDFTLKISRVEAEDLGVYHCWQGTHFPYTFGGGTKLEIKRADAAP
TVSX

This suggested that the 8B2-H6 clone might not be derived from a single cell. Therefore, the cells from this clone were re-plated in serial dilution (one cell per well, in a 96 well plate), to isolate a single cell clone producing the mutant CALR specific monoclonal antibody. The clone 8B2-H6-10.7 was used to extract RNA, prepare cDNA and amplify the variable regions of the immunoglobulin heavy chain and light chain. The exact same light chain sequence and two heavy chain sequences were obtained, suggesting that the clone is derived from a single cell, but produces two functional antibodies composed of unique heavy chains, but the same light chain. The antibody from the supernatant of the clone 8B2-H6-10.7 was purified and concentrated by binding to HiTrap™ Protein G HP column and the antibody was eluted into different fractions. Western blot analysis showed that the Fraction 4 of the eluted fractions contained the most concentrated levels of the antibody (FIG. 7). However, the signal is not very specific due to presence of the two heavy chains.

The 8B2-H6-10.7 (fraction 4) was used to stain Ba/F3-MPL cells expressing the different CALR constructs for detection of the surface CALR by FACS analysis. Anti-mouse PE antibody was used as secondary antibody. FIG. 8 shows specific detection of mutant CALR proteins, both del52 (Type1) and ins5 (Type2), on the surface of the respective Ba/F3 cells. Ba/F3-MPL cells expressing mutant CALR del52 (Type1) and ins5 (Type2) proteins showed a mild shift upon treatment with the antibody obtained from hybridoma 8B2-H6-10.7 and the secondary anti-mouse APC antibody compared to the non-treated control (“MPL”) and compared to the Ba/F3-MPL cells expressing wild-type CALR that were also treated with the antibody obtained from hybridoma 8B2-H6-10.7 and the secondary APC antibody. It is common in FACS that adding the secondary antibody creates a mild shift even if the primary antibody is highly specific for the antigen. This experiment shows that the antibody obtained from hybridoma 8B2-H6-10.7 binds indeed specifically to mutant calreticulin, but not to wild-type calreticulin.

The hybridoma clone 8B2-H6-10.7 has been deposited to DSMZ under the accession number DSM ACC3249.

To dissect the antibody specific to mutant CALR, the entire coding region of the light chain (with constant region of mouse kappa) and of the two heavy chain sequences (with constant region of mouse IgG2a) were synthesized (by Genscript) into pEE12.4 and pEE6.4, respectively. The complementarity determining regions (CDRs) are shown in bold letters.

IgG2a_H1:
DNA sequence-
AAGCTTGCCGCCACCATGGGATGGTCTTGTATTATTCTGTTTCTGGTCGC
CACCGCCACAGGAGTGCATTCCGAAGTCCAGCTGAAGCAGTCCGGCCCCG
AACTGGTCAAGACTGGCGCCAGTGTGAAAATCTCATGCAAGGCTAGCGGG
TACTCTTTCACCGGTTACTATATTCACTGGGTGAAACAGTCCCATGGCAA
GAGCCTGGAATGGATCGGATACATTTCTTGTTATAACGGGGCATCCAGCT
ACAATCAGAAGTTCAAAGGCAAGGCCACCTTTACAGTGGACACCTCTAGT
TCAACAGCTTATATGCAGTTTAACAGTCTGACATCAGGCGACTCCGCTGT
GTACTATTGCGCATCCAGCATGGATTACTGGGGGCAGGGTACATCCGTCA
CTGTGTCTAGTGCAAAGACCACAGCCCCCAGCGTCTATCCTCTGGCTCCA
GTGTGCGGCGATACTACCGGATCATCCGTCACTCTGGGCTGTCTGGTGAA
GGGATACTTCCCTGAGCCAGTGACTCTGACCTGGAACTCCGGGAGCCTGA
GCTCTGGTGTCCACACCTTTCCTGCCGTGCTGCAGTCTGACCTGTATACA
CTGAGTTCATCCGTCACAGTGACTAGCTCTACATGGCCTTCTCAGAGTAT
CACTTGCAACGTGGCCCATCCAGCTAGTTCAACAAAGGTGGATAAGAAAA
TCGAACCCCGGGGCCCTACCATCAAGCCATGTCCCCCTTGCAAGTGTCCC
GCTCCTAATCTGCTGGGCGGACCCTCCGTGTTCATCTTTCCACCCAAAAT
TAAGGACGTGCTGATGATCTCACTGTCCCCCATTGTCACCTGTGTGGTCG
TGGACGTGTCTGAGGACGATCCTGATGTCCAGATCTCCTGGTTCGTGAAC
AATGTCGAAGTGCACACCGCTCAGACCCAGACACATAGGGAGGATTACAA
CTCCACACTGCGGGTCGTGAGCGCACTGCCAATTCAGCACCAGGACTGGA
TGTCCGGAAAAGAGTTCAAGTGCAAGGTGAACAATAAGGATCTGCCAGCA
CCCATCGAGCGAACCATTTCTAAACCAAAGGGGAGTGTGCGTGCCCCCCA
GGTCTATGTGCTGCCTCCACCCGAGGAAGAGATGACTAAGAAACAGGTCA
CTCTGACCTGTATGGTGACCGACTTCATGCCTGAAGATATCTACGTGGAG
TGGACTAACAATGGAAAAACCGAACTGAACTATAAGAATACCGAGCCAGT
GCTGGACAGCGATGGGTCTTACTTTATGTATAGCAAGCTGAGAGTCGAAA
AGAAAAACTGGGTGGAGCGCAATAGCTACTCTTGCAGTGTCGTGCACGAG
GGTCTGCATAATCACCATACAACTAAATCATTCTCCCGCACACCCGGCAA
GTAATGAGAATTC
Protein sequence-
MGWSCIILFLVATATGVHSEVQLKQSGPELVKTGASVKISCKAS [symbol]
HWVKQSHGKSLEWIGY [symbol] SYNQKFKGKAT
FTVDTSSSTAYMQFNSLTSGDSAVYYC [symbol] [symbol] WGQGTSVTV
SSAKTTAPSVYPLAPVCGDTTGSSVTLGCLVKGYFPEPVTLTWN
SGSLSSGVHTFPAVLQSDLYTLSSSVTVTSSTWPSQSITCNVAH
PASSTKVDKKIEPRGPTIKPCPPCKCPAPNLLGGPSVFIFPPKI
KDVLMISLSPIVTCVVVDVSEDDPDVQISWFVNNVEVHTAQTQT
HREDYNSTLRVVSALPIQHQDWMSGKEFKCKVNNKDLPAPIERT
ISKPKGSVRAPQVYVLPPPEEEMTKKQVTLTCMVTDFMPEDIYV
EWTNNGKTELNYKNTEPVLDSDGSYFMYSKLRVEKKNWVERNSY
SCSVVHEGLHNHHTTKSFSRTPGK
IgG2a_H2:
DNA sequence-
AAGCTTGCCGCCACCATGGGTTGGTCTTGTATCATTCTGTTTCTGGTCGC
TACCGCTACTGGGGTCCATTCCGATGTGCAGCTGAAACTGCAGGAGTCTG
GGCCAGGGCTGGTGAAGAACAGTCAGTCACTGTCCCTGACCTGCACAGTG
ACTGGTTATAGCATCACTTCTGACTACGCCTGGAACTGGATTAGACAGTT
CCCCGGCAATAAGCTGGAATGGATGGGGTATATCAGCTACTCTGGTAGTA
CCTCATATAACCCTAGTCTGAAGTCAAGGATCTCCATTACCCGGGATACA
TCTAAAAACCAGTTCTTTCTGCAGCTGAACTCCGTGACACCTGAGGACAC
CGCTACATACTATTGTGCACGCGATCCCCCTTACTATTACGGGAGCAATG
GTACTCTGACCGTGTCCAGCGCAAAGACCACAGCCCCATCTGTCTATCCC
CTGGCTCCTGTGTGCGGCGACACTACCGGATCTAGTGTCACCCTGGGGTG
TCTGGTGAAGGGTTACTTCCCCGAGCCTGTGACACTGACTTGGAACTCCG
GCAGCCTGTCATCCGGAGTCCACACCTTTCCCGCAGTGCTGCAGTCCGAC
CTGTACACACTGAGCTCTAGTGTCACCGTGACATCATCCACATGGCCCTC
TCAGAGTATTACTTGCAACGTCGCCCATCCTGCTAGCTCTACAAAGGTGG
ATAAGAAAATCGAACCACGAGGCCCCACTATTAAGCCTTGTCCACCCTGC
AAATGTCCAGCTCCCAATCTGCTGGGCGGACCAAGCGTGTTCATCTTTCC
TCCAAAGATCAAGGACGTGCTGATGATCTCACTGTCCCCAATTGTCACCT
GCGTGGTCGTGGACGTGTCTGAGGACGATCCCGATGTCCAGATCAGTTGG
TTCGTGAACAATGTCGAAGTGCACACCGCACAGACTCAGACCCATAGAGA
GGATTATAACTCCACACTGCGAGTCGTGAGCGCACTGCCTATTCAGCACC
AGGACTGGATGTCTGGGAAGGAGTTCAAGTGCAAAGTGAACAACAAGGAT
CTGCCTGCCCCAATCGAGAGGACCATTAGTAAGCCTAAAGGATCAGTGCG
GGCTCCACAGGTCTACGTGCTGCCACCTCCAGAGGAAGAGATGACTAAGA
AACAGGTCACACTGACTTGTATGGTGACCGACTTCATGCCAGAAGATATC
TATGTGGAGTGGACTAACAATGGCAAGACCGAACTGAACTACAAAAATAC
AGAGCCCGTGCTGGACAGCGATGGATCTTATTTTATGTACAGCAAGCTGC
GAGTCGAAAAGAAAAACTGGGTGGAGCGTAATAGCTACTCTTGTAGTGTC
GTGCACGAGGGCCTGCATAATCACCATACAACTAAGTCATTTTCCCGGAC
TCCCGGAAAATAATGAGAATTC
Protein sequence-
MGWSCIILFLVATATGVHSDVQLKLQESGPGLVKNSQSLSLTCTVT [symbol]
AWNWIRQFPGNKLEWMGY [symbol] SYNPSLKSRISITRD
TSKNQFFLQLNSVTPEDTATYYCA [symbol] [symbol] LTVSSAKTTAPS
VYPLAPVCGDTTGSSVTLGCLVKGYFPEPVTLTWNSGSLSSGVHTFPAVL
QSDLYTLSSSVTVTSSTWPSQSITCNVAHPASSTKVDKKIEPRGPTIKPC
PPCKCPAPNLLGGPSVFIFPPKIKDVLMISLSPIVTCVVVDVSEDDPDVQ
ISWFVNNVEVHTAQTQTHREDYNSTLRVVSALPIQHQDWMSGKEFKCKVN
NKDLPAPIERTISKPKGSVRAPQVYVLPPPEEEMTKKQVTLTCMVTDFMP
EDIYVEWTNNGKTELNYKNTEPVLDSDGSYFMYSKLRVEKKNWVERNSYS
CSVVHEGLHNHHTTKSFSRTPGK
K_L1:
DNA sequence-
AAGCTTGCCGCCACCATGGGCTGGTCCTGTATTATCCTGTTCCTGGTCGC
TACTGCTACTGGGGTCCATTCCGATGTCGTGATGACTCAGACTCCACTGA
CTCTGTCCGTGACAATCGGGCAGCCCGCCAGCATTTCTTGCAAGTCCAGC
CAGTCCCTGCTGGACAGCGATGGCAAAACCTACCTGAACTGGCTGCTGCA
GAGGCCAGGACAGAGCCCCAAGCGGCTGATCTATCTGGTGTCTAAACTGG
ACAGTGGCGTCCCTGATAGATTCACCGGAAGTGGGTCAGGTACTGACTTT
ACCCTGAAGATTTCTCGCGTGGAGGCTGAAGATCTGGGGGTCTACCACTG
CTGGCAGGGTACCCATTTCCCTTATACATTTGGCGGAGGGACTAAGCTGG
AGATCAAACGGGCTGACGCCGCTCCAACTGTGTCCATTTTCCCCCCTTCT
AGTGAACAGCTGACCTCAGGTGGCGCATCCGTGGTCTGTTTCCTGAACAA
TTTTTACCCAAAGGACATCAACGTGAAGTGGAAAATTGATGGCAGCGAGC
GCCAGAACGGAGTGCTGAACTCCTGGACCGACCAGGATTCTAAGGACAGT
ACATATTCAATGTCATCCACCCTGACACTGACTAAAGATGAGTACGAACG
ACACAATAGTTATACATGTGAAGCAACTCATAAGACCTCCACAAGCCCCA
TCGTGAAATCCTTTAACCGTAATGCCTAATGAGAATTC
Protein sequence-
MGWSCIILFLVATATGVHSDVVMTQTPLTLSVTIGQPASISCKSS [symbol]
LNWLLQRPGQSPKRLIY [symbol] KLDSGVPDRFTGSGSGTD
FTLKISRVEAEDLGVYHC [symbol] [symbol] FGGGTKLEIKRADAAPTVSIF
PPSSEQLTSGGASVVCFLNNFYPKDINVKWKIDGSERQNGVLNSWTDQD
SKDSTYSMSSTLTLTKDEYERHNSYTCEATHKTSTSPIVKSFNRNA

CHO cells were transiently transfected with individual heavy and light chain expressing constructs by electroporation and the supernatant of the cells was used as probe for Western blotting. Interestingly, both H1L1 and H2L1 antibody recognized the mutant CALR specifically. However, the H2L1 antibody is very specific and does not show any low background (FIG. 9).

We have successfully generated a monoclonal antibody, specific to mutant CALR. This antibody can specifically bind to the mutant CALR, both in Western blot and FACS analysis. This antibody can be used as research reagent as well as for diagnostic purposes as disclosed herein.

The present invention refers to the following nucleotide and amino acid sequences:

Some sequences provided herein are available in the NCBI database and can be retrieved from world wide web at ncbi.nlm.nih.gov/sites/entrez?db=gene; Theses sequences also relate to annotated and modified sequences. The present invention also provides techniques and methods wherein homologous sequences, and variants of the concise sequences provided herein are used. Preferably, such “variants” are genetic variants.

SEQ ID No. 1:
Amino acid sequence of CDR-H1 of heavy chain H1
GYSFTGYY
SEQ ID No. 2:
Amino acid sequence of CDR-H2 of heavy chain H1
ISCYNGAS
SEQ ID No. 3:
Amino acid sequence of CDR-H3 of heavy chain H1
ASSMDY
SEQ ID No. 4:
Amino acid sequence of CDR-H1 of heavy chain H2
GYSITSDYA
SEQ ID No. 5:
Amino acid sequence of CDR-H2 of heavy chain H2
ISYSGST
SEQ ID No. 6:
Amino acid sequence of CDR-H3 of heavy chain H2
ARDPPYYYGSNGT
SEQ ID No. 7:
Amino acid sequence of CDR-L1 of light chain L1
QSLLDSDGKTY
SEQ ID No. 8:
Amino acid sequence of CDR-L2 of light chain L1
LVS
SEQ ID No. 9:
Amino acid sequence of CDR-L3 of light chain L1
WQGTHFPYT
SEQ ID No. 10:
Nucleic acid sequence encoding the variable V_{H}-region of heavy chain H1
ATATCCTGCAAGGCTTCTGGTTACTCTTTCACTGGTTACTACATACACTGGGTCAAG
CAGAGCCATGGAAAGAGCCTTGAGTGGATTGGATATATTAGTTGTTACAATGGTGC
TTCTAGCTACAACCAGAAGTTCAAGGGCAAGGCCACATTTACTGTAGACACATCCT
CCAGCACAGCCTACATGCAGTTCAACAGCCTGACATCTGGAGACTCTGCGGTCTAT
TACTGTGCAAGTTCTATGGACTACTGGGGTCAAGGAACCTCAGTCACCGTCTCCTC
AGCCAAAACGACACCCCCATCTGACTA
SEQ ID No. 11:
Amino acid sequence of the variable V_{H}-region of heavy chain H1. The complementarity
determining regions (CDRs) are highlighted in bold letters.
ISCKASGYSFTGYYIHWVKQSHGKSLEWIGYISCYNGASSYNQKFKGKATFTVDTSSS
TAYMQFNSLTSGDSAVYYCASSMDYWGQGTSVTVSSAKTTPPSD
SEQ ID No. 12:
Nucleic acid sequence encoding the variable V_{H}-region of heavy chain H2
TTGGCCCCAGTAGTCAAAGTAGTACCATTACTACCGTAGTAATAGGGGGGGTCTCT
TGCACAGTAATATGTGGCTGTGTCCTCAGGAGTCACAGAATTCAACTGCAGGAAG
AACTGGTTCTTGGATGTGTCTCGAGTGATAGAGATTCGACTTTTGAGAGATGGGTT
GTAGCTAGTGCTACCACTGTAGCTTATGTAGCCCATCCACTCCAGTTTGTTTCCTGG
AAACTGCCGGATCCAGTTCCAGGCATAATCACTGGTGATTGAGTAGCCAGTGACA
GTGCAGGTGAGGGACAGAGACTGAGAATTTTTCACCAGGCCAGGTCCCGACTCCT
GAAGCTTTCACATCA
SEQ ID No. 13:
Amino acid sequence of the variable V_{H}-region of heavy chain H2. The complementarity
determining regions (CDRs) are highlighted in bold letters.
KLQESGPGLVKNSQSLSLTCTVTGYSITSDYAWNWIRQFPGNKLEWMGYISYSGSTSY
NPSLKSRISITRDTSKNQFFLQLNSVTPEDTATYYCARDPPYYYGSNGTTLTTGA
SEQ ID No. 14:
Nucleic acid sequence encoding the variable V_{L}-region of light chain Ll
CAGCCTCCATCTCTTGCAAGTCAAGTCAGAGCCTCTTAGATAGTGATGGAAAGACA
TATTTGAATTGGTTGTTACAGAGGCCAGGCCAGTCTCCAAAGCGCCTAATCTATCT
GGTGTCTAAACTGGACTCTGGAGTCCCTGACAGGTTCACTGGCAGTGGATCAGGGA
CAGATTTCACACTGAAAATCAGCAGAGTGGAGGCTGAGGATTTGGGAGTTTATCAT
TGCTGGCAAGGTACACATTTTCCGTACACGTTCGGAGGGGGGACCAAGCTGGAAA
TAAAACGGGCTGATGCTGCACCAACTGTATCCN
SEQ ID No. 15:
Amino acid sequence of the variable V_{L}-region of light chain L1. The complementarity
determining regions (CDRs) are highlighted in bold letters.
ASISCKSSQSLLDSDGKTYLNWLLQRPGQSPKRLIYLVSKLDSGVPDRFTGSGSGTDFT
LKISRVEAEDLGVYHCWQGTHFPYTFGGGTKLEIKRADAAPTVSX
SEQ ID No. 16:
Nucleic acid sequence encoding heavy chain H1 (isotype IgG2a)
AAGCTTGCCGCCACCATGGGATGGTCTTGTATTATTCTGTTTCTGGTCGCCACCGCC
ACAGGAGTGCATTCCGAAGTCCAGCTGAAGCAGTCCGGCCCCGAACTGGTCAAGA
CTGGCGCCAGTGTGAAAATCTCATGCAAGGCTAGCGGGTACTCTTTCACCGGTTAC
TATATTCACTGGGTGAAACAGTCCCATGGCAAGAGCCTGGAATGGATCGGATACA
TTTCTTGTTATAACGGGGCATCCAGCTACAATCAGAAGTTCAAAGGCAAGGCCACC
TTTACAGTGGACACCTCTAGTTCAACAGCTTATATGCAGTTTAACAGTCTGACATC
AGGCGACTCCGCTGTGTACTATTGCGCATCCAGCATGGATTACTGGGGGCAGGGTA
CATCCGTCACTGTGTCTAGTGCAAAGACCACAGCCCCCAGCGTCTATCCTCTGGCT
CCAGTGTGCGGCGATACTACCGGATCATCCGTCACTCTGGGCTGTCTGGTGAAGGG
ATACTTCCCTGAGCCAGTGACTCTGACCTGGAACTCCGGGAGCCTGAGCTCTGGTG
TCCACACCTTTCCTGCCGTGCTGCAGTCTGACCTGTATACACTGAGTTCATCCGTCA
CAGTGACTAGCTCTACATGGCCTTCTCAGAGTATCACTTGCAACGTGGCCCATCCA
GCTAGTTCAACAAAGGTGGATAAGAAAATCGAACCCCGGGGCCCTACCATCAAGC
CATGTCCCCCTTGCAAGTGTCCCGCTCCTAATCTGCTGGGCGGACCCTCCGTGTTCA
TCTTTCCACCCAAAATTAAGGACGTGCTGATGATCTCACTGTCCCCCATTGTCACCT
GTGTGGTCGTGGACGTGTCTGAGGACGATCCTGATGTCCAGATCTCCTGGTTCGTG
AACAATGTCGAAGTGCACACCGCTCAGACCCAGACACATAGGGAGGATTACAACT
CCACACTGCGGGTCGTGAGCGCACTGCCAATTCAGCACCAGGACTGGATGTCCGG
AAAAGAGTTCAAGTGCAAGGTGAACAATAAGGATCTGCCAGCACCCATCGAGCGA
ACCATTTCTAAACCAAAGGGGAGTGTGCGTGCCCCCCAGGTCTATGTGCTGCCTCC
ACCCGAGGAAGAGATGACTAAGAAACAGGTCACTCTGACCTGTATGGTGACCGAC
TTCATGCCTGAAGATATCTACGTGGAGTGGACTAACAATGGAAAAACCGAACTGA
ACTATAAGAATACCGAGCCAGTGCTGGACAGCGATGGGTCTTACTTTATGTATAGC
AAGCTGAGAGTCGAAAAGAAAAACTGGGTGGAGCGCAATAGCTACTCTTGCAGTG
TCGTGCACGAGGGTCTGCATAATCACCATACAACTAAATCATTCTCCCGCACACCC
GGCAAGTAATGAGAATTC
SEQ ID No. 17:
Amino acid sequence of heavy chain H1 (isotype IgG2a). The complementarity determining
regions (CDRs) are highlighted in bold letters. The constant region is underlined.
MGWSCIILFLVATATGVHSEVQLKQSGPELVKTGASVKISCKAS [symbol] IHWVKQ
SHGKSLEWIGY [symbol] SYNQKFKGKATFTVDTSSSTAYMQFNSLTSGDSAVYYC [symbol] [symbol]
WGQGTSVTVSSAKTTAPSVYPLAPVCGDTTGSSVTLGCLVKGYFPEPVTLTW
NSGSLSSGVHTFPAVLQSDLYTLSSSVTVTSSTWPSQSITCNVAHPASSTKVDKKIEPRG
PTIKPCPPCKCPAPNLLGGPSVFIFPPKIKDVLMISLSPIVTCVVVDVSEDDPDVQISWFV
NNVEVHTAQTQTHREDYNSTLRVVSALPIQHQDWMSGKEFKCKVNNKDLPAPIERTIS
KPKGSVRAPQVYVLPPPEEEMTKKQVTLTCMVTDFMPEDIYVEWTNNGKTELNYKNT
EPVLDSDGSYFMYSKLRVEKKNWVERNSYSCSVVHEGLHNHHTTKSFSRTPGK
SEQ ID No. 18:
Nucleic acid sequence encoding heavy chain H2 (isotype IgG2a)
AAGCTTGCCGCCACCATGGGTTGGTCTTGTATCATTCTGTTTCTGGTCGCTACCGCT
ACTGGGGTCCATTCCGATGTGCAGCTGAAACTGCAGGAGTCTGGGCCAGGGCTGG
TGAAGAACAGTCAGTCACTGTCCCTGACCTGCACAGTGACTGGTTATAGCATCACT
TCTGACTACGCCTGGAACTGGATTAGACAGTTCCCCGGCAATAAGCTGGAATGGAT
GGGGTATATCAGCTACTCTGGTAGTACCTCATATAACCCTAGTCTGAAGTCAAGGA
TCTCCATTACCCGGGATACATCTAAAAACCAGTTCTTTCTGCAGCTGAACTCCGTG
ACACCTGAGGACACCGCTACATACTATTGTGCACGCGATCCCCCTTACTATTACGG
GAGCAATGGTACTCTGACCGTGTCCAGCGCAAAGACCACAGCCCCATCTGTCTATC
CCCTGGCTCCTGTGTGCGGCGACACTACCGGATCTAGTGTCACCCTGGGGTGTCTG
GTGAAGGGTTACTTCCCCGAGCCTGTGACACTGACTTGGAACTCCGGCAGCCTGTC
ATCCGGAGTCCACACCTTTCCCGCAGTGCTGCAGTCCGACCTGTACACACTGAGCT
CTAGTGTCACCGTGACATCATCCACATGGCCCTCTCAGAGTATTACTTGCAACGTC
GCCCATCCTGCTAGCTCTACAAAGGTGGATAAGAAAATCGAACCACGAGGCCCCA
CTATTAAGCCTTGTCCACCCTGCAAATGTCCAGCTCCCAATCTGCTGGGCGGACCA
AGCGTGTTCATCTTTCCTCCAAAGATCAAGGACGTGCTGATGATCTCACTGTCCCC
AATTGTCACCTGCGTGGTCGTGGACGTGTCTGAGGACGATCCCGATGTCCAGATCA
GTTGGTTCGTGAACAATGTCGAAGTGCACACCGCACAGACTCAGACCCATAGAGA
GGATTATAACTCCACACTGCGAGTCGTGAGCGCACTGCCTATTCAGCACCAGGACT
GGATGTCTGGGAAGGAGTTCAAGTGCAAAGTGAACAACAAGGATCTGCCTGCCCC
AATCGAGAGGACCATTAGTAAGCCTAAAGGATCAGTGCGGGCTCCACAGGTCTAC
GTGCTGCCACCTCCAGAGGAAGAGATGACTAAGAAACAGGTCACACTGACTTGTA
TGGTGACCGACTTCATGCCAGAAGATATCTATGTGGAGTGGACTAACAATGGCAA
GACCGAACTGAACTACAAAAATACAGAGCCCGTGCTGGACAGCGATGGATCTTAT
TTTATGTACAGCAAGCTGCGAGTCGAAAAGAAAAACTGGGTGGAGCGTAATAGCT
ACTCTTGTAGTGTCGTGCACGAGGGCCTGCATAATCACCATACAACTAAGTCATTT
TCCCGGACTCCCGGAAAATAATGAGAATTC
SEQ ID No. 19:
Amino acid sequence of heavy chain H2 (isotype IgG2a). The complementarity determining
regions (CDRs) are highlighted in bold letters. The constant region is underlined.
MGWSCIILFLVATATGVHSDVQLKLQESGPGLVKNSQSLSLTCTVT [symbol] WNWI
RQFPGNKLEWMGY [symbol] SYNPSLKSRISITRDTSKNQFFLQLNSVTPEDTATYYC [symbol] [symbol]
LTVSSAKTTAPSVYPLAPVCGDTTGSSVTLGCLVKGYFPEPVTLTWNS
GSLSSGVHTFPAVLQSDLYTLSSSVTVTSSTWPSQSITCNVAHPASSTKVDKKIEPRGPT
IKPCPPCKCPAPNLLGGPSVFIFPPKIKDVLMISLSPIVTCVVVDVSEDDPDVQISWFVNN
VEVHTAQTQTHREDYNSTLRVVSALPIQHQDWMSGKEFKCKVNNKDLPAPIERTISKP
KGSVRAPQVYVLPPPEEEMTKKQVTLTCMVTDFMPEDIYVEWTNNGKTELNYKNTEP
VLDSDGSYFMYSKLRVEKKNWVERNSYSCSVVHEGLHNHHTTKSFSRTPGK
SEQ ID No. 20:
Nucleic acid sequence encoding light chain L1
AAGCTTGCCGCCACCATGGGCTGGTCCTGTATTATCCTGTTCCTGGTCGCTACTGCT
ACTGGGGTCCATTCCGATGTCGTGATGACTCAGACTCCACTGACTCTGTCCGTGAC
AATCGGGCAGCCCGCCAGCATTTCTTGCAAGTCCAGCCAGTCCCTGCTGGACAGCG
ATGGCAAAACCTACCTGAACTGGCTGCTGCAGAGGCCAGGACAGAGCCCCAAGCG
GCTGATCTATCTGGTGTCTAAACTGGACAGTGGCGTCCCTGATAGATTCACCGGAA
GTGGGTCAGGTACTGACTTTACCCTGAAGATTTCTCGCGTGGAGGCTGAAGATCTG
GGGGTCTACCACTGCTGGCAGGGTACCCATTTCCCTTATACATTTGGCGGAGGGAC
TAAGCTGGAGATCAAACGGGCTGACGCCGCTCCAACTGTGTCCATTTTCCCCCCTT
CTAGTGAACAGCTGACCTCAGGTGGCGCATCCGTGGTCTGTTTCCTGAACAATTTT
TACCCAAAGGACATCAACGTGAAGTGGAAAATTGATGGCAGCGAGCGCCAGAACG
GAGTGCTGAACTCCTGGACCGACCAGGATTCTAAGGACAGTACATATTCAATGTCA
TCCACCCTGACACTGACTAAAGATGAGTACGAACGACACAATAGTTATACATGTG
AAGCAACTCATAAGACCTCCACAAGCCCCATCGTGAAATCCTTTAACCGTAATGCC
TAATGAGAATTC
SEQ ID No. 21:
Amino acid sequence of light chain L1. The complementarity determining regions
(CDRs) are highlighted in bold letters. The constant region is underlined.
MGWSCIILFLVATATGVHSDVVMTQTPLTLSVTIGQPASISCKSS [symbol] LNW
LLQRPGQSPKRLIY [symbol] LDSGVPDRFTGSGSGTDFTLKISRVEAEDLGVYHC [symbol] [symbol]
FGGGTKLEIKRADAAPTVSIFPPSSEQLTSGGASVVCFLNNFYPKDINVKWKIDGSE
RQNGVLNSWTDQDSKDSTYSMSSTLTLTKDEYERHNSYTCEATHKTSTSPIVKSFNRN
A

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. An antibody that specifically binds to a mutant calreticulin protein,
wherein the variable region of the heavy chain of said antibody comprises a CDR-H3 region having an amino acid sequence as depicted in SEQ ID NO.: 3, or a CDR sequence having 75% or more amino acid identity to said CDR;
or
wherein the variable region of the heavy chain of said antibody comprises a CDR-H3 region having an amino acid sequence as depicted in SEQ ID NO.: 6, or a CDR sequence having 75% or more amino acid identity to said CDR.

2. The antibody of claim 1,
wherein the variable region of the heavy chain of said antibody comprises a CDR-H1 region having an amino acid sequence as depicted in SEQ ID NO: 1, or a CDR sequence having 75% or more amino acid identity to said CDR;
or
wherein the variable region of the heavy chain of said antibody comprises a CDR-H1 region having an amino acid sequence as depicted in SEQ ID NO: 4, or a CDR sequence having 75% or more amino acid identity to said CDR.

3. The antibody of claim 1 or 2, wherein the variable region of the heavy chain of said antibody comprises a CDR-H2 region having an amino acid sequence as depicted in SEQ ID NO: 2, or a CDR sequence having 75% or more amino acid identity to said CDR;
or
wherein the variable region of the heavy chain of said antibody comprises a CDR-H2 region having an amino acid sequence as depicted in SEQ ID NO: 5, or a CDR sequence having 75% or more amino acid identity to said CDR.

4. An antibody that specifically binds to a mutant calreticulin protein, wherein the variable region of the heavy chain of said antibody comprises a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3, or a CDR sequence having 75% or more amino acid identity to one of said CDRs.

5. The antibody of claim 4, wherein the variable region of the heavy chain of said antibody comprises a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3.

6. An antibody that specifically binds to a mutant calreticulin protein, wherein the variable region of the heavy chain of said antibody comprises a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6, or a CDR sequence having 75% or more amino acid identity to one of said CDRs.

7. The antibody of claim 6, wherein the variable region of the heavy chain of said antibody comprises a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6.

8. The antibody of any one of claims 1 to 7, wherein the variable region of the light chain of said antibody comprises a CDR-L1 region having an amino acid sequence as depicted in SEQ ID NO: 7, or a CDR sequence having 75% or more amino acid identity to said CDR.

9. The antibody of any one of claims 1 to 8, wherein the variable region of the light chain of said antibody comprises a CDR-L2 region having an amino acid sequence as depicted in SEQ ID NO: 8, or a CDR sequence having 75% or more amino acid identity to said CDR.

10. The antibody of any one of claims 1 to 9, wherein the variable region of the light chain of said antibody comprises a CDR-L3 region having an amino acid sequence as depicted in SEQ ID NO: 9, or a CDR sequence having 75% or more amino acid identity to said CDR.

11. The antibody of any one of claims 1 to 7, wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

12. An antibody that specifically binds to a mutant calreticulin protein, wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

13. The antibody of claim 11 or 12, wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

14. An antibody that specifically binds to a mutant calreticulin protein,
wherein the variable region of the heavy chain of said antibody comprises a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3, or a CDR sequence having 75% or more amino acid identity to one of said CDRs;
and
wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

15. The antibody of claim 14,
wherein the variable region of the heavy chain of said antibody comprises a CDR-H1 region having an amino acid sequence as depicted in SEQ ID NO: 1, a CDR-H2 region having an amino acid sequence as depicted in SEQ ID NO: 2, and a CDR-H3 region having an amino acid sequence as depicted in SEQ ID NO.: 3;
and
wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

16. An antibody that specifically binds to a mutant calreticulin protein,
wherein the variable region of the heavy chain of said antibody comprises a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6, or a CDR sequence having 75% or more amino acid identity to one of said CDRs;
and
wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9, or a CDR sequence having 75% or more amino acid identity to one of said CDRs.

17. The antibody of claim 16,
wherein the variable region of the heavy chain of said antibody comprises a CDR-H1 region having an amino acid sequence as depicted in SEQ ID NO: 4, a CDR-H2 region having an amino acid sequence as depicted in SEQ ID NO: 5, and a CDR-H3 region having an amino acid sequence as depicted in SEQ ID NO.: 6;
and
wherein the variable region of the light chain of said antibody comprises a CDR-L1 region having an amino acid sequence as depicted in SEQ ID NO: 7, a CDR-L2 region having an amino acid sequence as depicted in SEQ ID NO: 8, and a CDR-L3 region having an amino acid sequence as depicted in SEQ ID NO: 9.

18. The antibody of any one of claims 1 to 17,
wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region

19. An antibody that specifically binds to a mutant calreticulin protein,
wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region.

20. The antibody of claim 18 or 19,
wherein said antibody comprises a variable V_{H}-region as encoded by a nucleic acid molecule as shown in SEQ ID NO:10; or
a variable V_{H}-region having an amino acid sequence as shown in SEQ ID NO:11.

21. The antibody of any one of claims 1 to 17,
wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region.

22. An antibody that specifically binds to a mutant calreticulin protein,
wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region.

23. The antibody of claim 21 or 22,
wherein said antibody comprises a variable V_{H}-region as encoded by a nucleic acid molecule as shown in SEQ ID NO:12; or
a variable V_{H}-region having an amino acid sequence as shown in SEQ ID NO:13.

24. The antibody of any one of claims 1 to 23,
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region.

25. An antibody that specifically binds to a mutant calreticulin protein,
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region.

26. The antibody of claim 24 or 25,
wherein said antibody comprises a variable V_{L}-region as encoded by a nucleic acid molecule as shown in SEQ ID NO:14, or
a variable V_{L}-region having an amino acid sequence as shown in SEQ ID NO:15.

27. An antibody that specifically binds to a mutant calreticulin protein,
wherein said antibody comprises a variable V_{H}-region as encoded by a nucleic acid molecule as shown in SEQ ID NO:10, or a variable V_{H}-region as encoded by a nucleic acid molecule having 75% or more identity to said variable V_{H}-region; or
a variable V_{H}-region having an amino acid sequence as shown in SEQ ID NO:11, or a variable V_{H}-region having an amino acid sequence which has 75% or more identity to said variable V_{H}-region;
and
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region or
a variable V_{L}-region having an amino acid sequence as shown in SEQ ID NO:15, or a variable V_{L}-region having an amino acid sequence which has 75% or more identity to said variable V_{L}-region.

28. The antibody of claim 27,
wherein said antibody comprises a variable V_{H}-region as encoded by a nucleic acid molecule as shown in SEQ ID NO:10; or
a variable V_{H}-region having an amino acid sequence as shown in SEQ ID NO:11;
and
wherein said antibody comprises a variable V_{L}-region as encoded by a nucleic acid molecule as shown in SEQ ID NO:14, or
a variable V_{L}-region having an amino acid sequence as shown in SEQ ID NO:15.

29. An antibody that specifically binds to a mutant calreticulin protein,
wherein said antibody comprises a variable V_{H}-region as encoded by a nucleic acid molecule as shown in SEQ ID NO:12, or a variable V_{H}-region as encoded by a nucleic acid molecule having 75% or more identity to said variable V_{H}-region; or
a variable V_{H}-region having an amino acid sequence as shown in SEQ ID NO:13, or a variable V_{H}-region having an amino acid sequence which has 75% or more identity to said variable V_{H}-region;
and
wherein said antibody comprises a variable V_{L}-region as encoded by a nucleic acid molecule as shown in SEQ ID NO:14, or a variable V_{L}-region as encoded by a nucleic acid molecule having 75% or more identity to said variable V_{L}-region; or
a variable V_{L}-region having an amino acid sequence as shown in SEQ ID NO:15, or a variable V_{L}-region having an amino acid sequence which has 75% or more identity to said variable V_{L}-region.

30. The antibody of claim 29,
wherein said antibody comprises a variable V_{H}-region as encoded by a nucleic acid molecule as shown in SEQ ID NO:12; or
a variable V_{H}-region having an amino acid sequence as shown in SEQ ID NO:13; a
and
wherein said antibody comprises a variable V_{L}-region as encoded by a nucleic acid molecule as shown in SEQ ID NO:14, or
a variable V_{L}-region having an amino acid sequence as shown in SEQ ID NO:15.

31. The antibody of any one of claims 1 to 30,
wherein said antibody comprises a heavy chain as encoded by a nucleic acid molecule as shown in SEQ ID NO:16, or a heavy chain as encoded by a nucleic acid molecule having 75% or more identity to said heavy chain; or
a heavy chain having an amino acid sequence as shown in SEQ ID NO:17, or a heavy chain having an amino acid sequence which has 75% or more identity to said heavy chain.

32. An antibody that specifically binds to a mutant calreticulin protein,
wherein said antibody comprises a heavy chain as encoded by a nucleic acid molecule as shown in SEQ ID NO:16, or a heavy chain as encoded by a nucleic acid molecule having 75% or more identity to said heavy chain; or
a heavy chain having an amino acid sequence as shown in SEQ ID NO:17, or a heavy chain having an amino acid sequence which has 75% or more identity to said heavy chain.

33. The antibody of claim 31 or 32,
wherein said antibody comprises a heavy chain as encoded by a nucleic acid molecule as shown in SEQ ID NO:16; or
a heavy chain having an amino acid sequence as shown in SEQ ID NO:17.

34. The antibody of any one of claims 1 to 30,
wherein said antibody comprises a heavy chain as encoded by a nucleic acid molecule as shown in SEQ ID NO:18, or a heavy chain as encoded by a nucleic acid molecule having 75% or more identity to said heavy chain; or
a heavy chain having an amino acid sequence as shown in SEQ ID NO:19, or a heavy chain having an amino acid sequence which has 75% or more identity to said heavy chain.

35. An antibody that specifically binds to a mutant calreticulin protein,
wherein said antibody comprises a heavy chain as encoded by a nucleic acid molecule as shown in SEQ ID NO:18, or a heavy chain as encoded by a nucleic acid molecule having 75% or more identity to said heavy chain; or
a heavy chain having an amino acid sequence as shown in SEQ ID NO:19, or a heavy chain having an amino acid sequence which has 75% or more identity to said heavy chain.

36. The antibody of claim 34 or 35,
wherein said antibody comprises a heavy chain as encoded by a nucleic acid molecule as shown in SEQ ID NO:18; or
a heavy chain having an amino acid sequence as shown in SEQ ID NO:19.

37. The antibody of any one of claims 1 to 36,
wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

38. An antibody that specifically binds to a mutant calreticulin protein,
wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

39. The antibody of claim 37 or 38,
wherein said antibody comprises a light chain as encoded by a nucleic acid molecule as shown in SEQ ID NO:20; or
a variable V_{H}-region having an amino acid sequence as shown in SEQ ID NO:21.

40. An antibody that specifically binds to a mutant calreticulin protein,
wherein said antibody comprises a heavy chain as encoded by a nucleic acid molecule as shown in SEQ ID NO:16, or a heavy chain as encoded by a nucleic acid molecule having 75% or more identity to said heavy chain; or
a heavy chain having an amino acid sequence as shown in SEQ ID NO:17, or a heavy chain having an amino acid sequence which has 75% or more identity to said heavy chain;
and
wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

41. The antibody of claim 40,
wherein said antibody comprises a heavy chain as encoded by a nucleic acid molecule as shown in SEQ ID NO:16; or
a heavy chain having an amino acid sequence as shown in SEQ ID NO:17;
and
wherein said antibody comprises a light chain as encoded by a nucleic acid molecule as shown in SEQ ID NO:20; or
a variable V_{H}-region having an amino acid sequence as shown in SEQ ID NO:21.

42. An antibody that specifically binds to a mutant calreticulin protein,
wherein said antibody comprises a heavy chain as encoded by a nucleic acid molecule as shown in SEQ ID NO:18, or a heavy chain as encoded by a nucleic acid molecule having 75% or more identity to said heavy chain; or
a heavy chain having an amino acid sequence as shown in SEQ ID NO:19, or a heavy chain having an amino acid sequence which has 75% or more identity to said heavy chain;
and
wherein said antibody comprises a light chain as encoded by a nucleic acid molecule as shown in SEQ ID NO:20, or a variable V_{H}-region as encoded by a nucleic acid molecule having 75% or more identity to said variable V_{H}-region; or
a variable V_{H}-region having an amino acid sequence as shown in SEQ ID NO:21, or a light chain having an amino acid sequence which has 75% or more identity to said variable V_{H}-region.

43. The antibody of claim 42,
wherein said antibody comprises a heavy chain as encoded by a nucleic acid molecule as shown in SEQ ID NO:18; or
a heavy chain having an amino acid sequence as shown in SEQ ID NO:19;
and
wherein said antibody comprises a light chain as encoded by a nucleic acid molecule as shown in SEQ ID NO:20; or
a variable V_{H}-region having an amino acid sequence as shown in SEQ ID NO:21.

44. An antibody that specifically binds to a mutant calreticulin protein,
wherein said antibody is obtained or obtainable from hybridoma 8B2-H6-10.7 deposited under accession number DSM ACC3249 with the depositary institute DSMZ on Sep. 12, 2014.

45. An antibody that binds to the same epitope as the antibody of any one of claims 1 to 44;
or an antibody having the same biological activity as the antibody of any one of claims 1 to 44.

46. The antibody of any one of claims 1 to 45, wherein said antibody is a murine antibody.

47. The antibody of claim 46, wherein said murine antibody is an IgG2a immunoglobulin.

48. The antibody of any one of claims 1 to 30 and 45, wherein said antibody is a full antibody (immunoglobulin), an antibody fragment such as a F(ab)-fragment or a F(ab)²-fragment, a single-chain antibody, a murine antibody, a chimeric antibody, a humanized antibody, a human antibody, a fully human antibody, a CDR-grafted antibody, a bivalent antibody-construct, a bispecific single-chain antibody, a synthetic antibody or a cross-cloned antibody.

49. The antibody of any one of claims 1 to 30 and 45, wherein said antibody is a humanized antibody or a human antibody.

50. The antibody of claim 49, wherein said antibody is an immunoglobulin selected from the group consisting of IgA, IgD, IgE, IgG or IgM.

51. The antibody that specifically binds to a mutant calreticulin protein of any one of claims 1 to 50, wherein the antibody specifically binds to the C-terminal part of mutant calreticulin protein or to a part of the C-terminal part of mutant calreticulin protein.

52. The antibody of claim 51, wherein the C-terminal part of mutant calreticulin protein is shown in any one of SEQ ID NOs: 35 to 70.

53. The antibody of claim 51, wherein the part of the C-terminal part of mutant calreticulin protein is shown in SEQ ID NO: 71.

54. A nucleic acid molecule having a sequence encoding the antibody as defined in any one of claims 1 to 53.

55. A vector comprising a nucleic acid molecule according to claim 53.

56. The vector of claim 55, which further comprises a nucleic acid molecule having a regulatory sequence which is operably linked to said nucleic acid molecule according to claim 54.

57. The vector of claim 55 or 56, wherein the vector is an expression vector.

58. A host transformed or transfected with a vector according to any of claims 55 to 57.

59. The host of claim 58, wherein said host is a eukaryotic host cell like COS, CHO, HEK293 or a multiple myeloma host cell.

60. Hybridoma 8B2-H6-10.7 deposited under accession number DSM ACC3249 with the depositary institute DSMZ on Sep. 12, 2014.

61. A process for the production of the antibody as defined in any one of items 1 to 50, said process comprising culturing a host of claim 58 or 59 or the hybridoma of claim 60 under conditions allowing the expression of the antibody and recovering the produced antibody from the culture.

62. A composition comprising the antibody as defined in any one of items 1 to 53 or as produced by the process of claim 61, a nucleic acid molecule of claim 54, a vector of any one of claims 55 to 57, a host of claim 58 or 59 and/or the hybridoma of claim 60.

63. The composition of claim 62, further comprising a secondary antibody that is specifically binding to the primary antibody as defined in any one of claims 1 to 53.

64. The composition of claim 62 or 63, which is a diagnostic composition further comprising, optionally, means and methods for detection.

65. A method for diagnosing a myeloid malignancy, comprising detecting or assaying a mutant calreticulin protein in a biological sample of an individual suspected of suffering from a myeloid malignancy or suspected of being prone to suffering from a myeloid malignancy using the antibody of any one of claims 1 to 53 or an antibody specifically binding to mutant calreticulin protein.

66. The method of claim 65, wherein the antibody specifically binds to the C-terminal part of mutant calreticulin protein or to a part of the C-terminal part of mutant calreticulin protein.

67. The method of claim 66, wherein the C-terminal part of mutant calreticulin protein is shown in any one of SEQ ID NOs: 35 to 70.

68. The method of claim 67, wherein the part of the C-terminal part of mutant calreticulin protein is shown in SEQ ID NO: 71.

69. The method of any one of claims 65 to 68, wherein the biological sample is a blood sample, a bone marrow sample or a serum sample.

70. The method of any one of claims 65 to 69, wherein the detection or the assay of mutant calreticulin protein is performed using immunologic methodologies, such as immunohistochemistry (IHC), immunocytochemistry, Western blot, or ELISA immunoassay; gel- or blot-based methods; mass spectrometry; flow cytometry; or fluorescent activated cell sorting (FACS).

71. The method of any one of claims 65 to 68, wherein said mutant calreticulin protein is present on the extracellular side of a plasma membrane of a cell.

72. The method of any one of claims 65 to 68, wherein said mutant calreticulin protein is present on surface of a cell.

73. The method of any one of claims 65 to 68, wherein said mutant calreticulin protein is localized at the extracellular side of a plasma membrane.

74. The method of any one of claims 65 to 68 and 71 to 73, wherein the cell is a living cell, whole cell or intact cell.

75. The method of any one of claims 65 to 68 and 71 to 73, wherein the detection or the assay of mutant calreticulin protein is performed using a flow cytometry technique.

76. The method of claim 75, wherein said flow cytometry technique is fluorescent activated cell sorting (FACS).

77. The method of any one of 65 to 68 and 71 to 76, wherein the biological sample is a blood sample or a bone marrow sample.

78. Use of the antibody of any one of claims 1 to 53 or as produced by the process of claim 61, the use of the nucleic acid molecule of claim 54, the use of the vector of any one of claims 55 to 57, the use of the host of claim 58 or 61 and/or the use of the hybridoma of claim 60 for the preparation of a diagnostic composition for the diagnosis of a myeloid malignancy.

79. Use of the antibody of any one of claims 1 to 53 or as produced by the process of claim 61, use of the nucleic acid molecule of claim 54, the use of the vector of any one of claims 55 to 57, the use of the host of claim 58 or 59, the use of the hybridoma of claim 60 and/or the use of the composition of any one of claims 61 to 64 for the preparation of a diagnostic kit for the diagnosis of a myeloid malignancy.

80. The antibody of any one of claims 1 to 53 or as produced by the process of claim 61, the nucleic acid molecule of claim 54, the vector of any one of claims 55 to 57, the host of claim 58 or 59, the hybridoma of claim 60 and/or the composition of any one of claims 62 to 64 for use in the diagnosis of a myeloid malignancy.

81. Kit comprising the antibody of any one of claims 1 to 53 or as produced by the process of claim 61, the nucleic acid molecule of claim 54, the vector of any one of claims 55 to 57, the host of claim 58 or 59, the hybridoma of claim 60 and/or the composition of any one of claims 62 to 64.

82. Use of the kit of claim 81 in the diagnosis of a myeloid malignancy.

83. The composition of claim 62 or 63, which is a pharmaceutical composition, optionally further comprising one or more pharmaceutically acceptable excipient(s).

84. The antibody of any one of claims 1 to 53 or as produced by the process of claim 61, the nucleic acid molecule of claim 54, the vector of any one of claims 55 to 57, the host of claim 58 or 59, the hybridoma of claim 60 and/or the composition of any one of claims 62, 63 and 83 for use in medicine.

85. Use of the antibody of any one of claims 1 to 53 or as produced by the process of claim 61, the nucleic acid molecule of claim 54, the vector of any one of claims 55 to 57, the host of claim 58 or 59, the hybridoma of claim 60 and/or the composition of claim 61 or 63 for the preparation of a pharmaceutical composition for the treatment of a myeloid malignancy.

86. The antibody of any one of claims 1 to 53 or as produced by the process of claim 61, the nucleic acid molecule of claim 54, the vector of any one of claims 55 to 57, the host of claim 58 or 59, the hybridoma of claim 60 and/or the composition of any one of claims 62, 63 and 83 for use in the treatment of a myeloid malignancy.

87. A method for the treatment of a myeloid malignancy comprising the administration of the antibody of any one of claims 1 to 53 or as produced by the process of claim 61, the nucleic acid molecule of claim 54, the vector of any one of claims 55 to 57, the host of claim 58 or 59, the hybridoma of claim 60 and/or the composition of any one of claims 62, 63 and 83 to a subject in need of such a treatment.

88. The method of claim 87, wherein said subject is a human.

89. The method of any one of claims 65 to 77, 87 and 88, the use of any one of claims 78, 79 and 85, the antibody of claim 80 or 86, the nucleic acid molecule of claim 80 or 86, the vector of claim 80 or 86, the host of claim 80 or 86 and/or the composition of claim 80 or 86, wherein said myeloid malignancy is a myeloproliferative neoplasm or a myelodysplastic syndrome.

90. The method of claim 89, the use of claim 89, the antibody of claim 89, the nucleic acid molecule of claim 89, the vector of claim 89, the host of claim 89 and/or the composition of claim 89, wherein said myeloproliferative neoplasm is selected from the group consisting of primary myelofibrosis (PMF) and essential thrombocythemia (ET).

91. The method of claim 89, the use of claim 89, the antibody of claim 89, the nucleic acid molecule of claim 89, the vector of claim 89, the host of claim 89 and/or the composition of claim 89, wherein said myelodysplastic syndrome is refractory anemia with ringed sideroblasts and thrombocythemia (RARS-T).


SEQUENCE LISTING (86 sequences)

SEQ ID NO: 1 - protein, 8 aa, Artificial Sequence
  CDR-H1 of heavy chain H1
GYSFTGYY

SEQ ID NO: 2 - protein, 8 aa, Artificial Sequence
  CDR-H2 of heavy chain H1
ISCYNGAS

SEQ ID NO: 3 - protein, 6 aa, Artificial Sequence
  CDR-H3 of heavy chain H1
ASSMDY

SEQ ID NO: 4 - protein, 9 aa, Artificial Sequence
  CDR-H1 of heavy chain H2
GYSITSDYA

SEQ ID NO: 5 - protein, 7 aa, Artificial Sequence
  CDR-H2 of heavy chain H2
ISYSGST

SEQ ID NO: 6 - protein, 13 aa, Artificial Sequence
  CDR-H3 of heavy chain H2
ARDPPYYYGSNGT

SEQ ID NO: 7 - protein, 11 aa, Artificial Sequence
  CDR-L1 of light chain L1
QSLLDSDGKTY

SEQ ID NO: 8 - protein, 3 aa, Artificial Sequence
  CDR-L2 of light chain L1
LVS

SEQ ID NO: 9 - protein, 9 aa, Artificial Sequence
  CDR-L3 of light chain L1
WQGTHFPYT

SEQ ID NO: 10 - DNA, 308 bp, Artificial Sequence
  variable VH-region of heavy chain H1
atatcctgcaaggcttctggttactctttcactggttactacatacactgggtcaagcagagccatggaaagagccttgagtggattggatatattagttgttacaatggtgcttctagctacaaccagaagttcaagggcaaggccacatttactgtagacacatcctccagcacagcctacatgcagttcaacagcctgacatctggagactctgcggtctattactgtgcaagttctatggactactggggtcaaggaacctcagtcaccgtctcctcagccaaaacgacacccccatctgacta

SEQ ID NO: 11 - protein, 102 aa, Artificial Sequence
  Variable VH-region of heavy chain H1
ISCKASGYSFTGYYIHWVKQSHGKSLEWIGYISCYNGASSYNQKFKGKATFTVDTSSSTAYMQFNSLTSGDSAVYYCASSMDYWGQGTSVTVSSAKTTPPSD

SEQ ID NO: 12 - DNA, 349 bp, Artificial Sequence
  Variable VH-region of heavy chain H2
ttggccccagtagtcaaagtagtaccattactaccgtagtaatagggggggtctcttgcacagtaatatgtggctgtgtcctcaggagtcacagaattcaactgcaggaagaactggttcttggatgtgtctcgagtgatagagattcgacttttgagagatgggttgtagctagtgctaccactgtagcttatgtagcccatccactccagtttgtttcctggaaactgccggatccagttccaggcataatcactggtgattgagtagccagtgacagtgcaggtgagggacagagactgagaatttttcaccaggccaggtcccgactcctgaagctttcacatca

SEQ ID NO: 13 - protein, 113 aa, Artificial Sequence
  Variable VH-region of heavy chain H2
KLQESGPGLVKNSQSLSLTCTVTGYSITSDYAWNWIRQFPGNKLEWMGYISYSGSTSYNPSLKSRISITRDTSKNQFFLQLNSVTPEDTATYYCARDPPYYYGSNGTTLTTGA

SEQ ID NO: 14 - DNA, 312 bp, Artificial Sequence
  Variable VL-region of light chain L1
  misc_feature 312: /note=“n = a, c, t or g”
cagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttatcattgctggcaaggtacacattttccgtacacgttcggaggggggaccaagctggaaataaaacgggctgatgctgcaccaactgtatccn

SEQ ID NO: 15 - protein, 104 aa, Artificial Sequence
  Variable VL-region of light chain L1
  UNSURE 104: Xaa = any amino acid
ASISCKSSQSLLDSDGKTYLNWLLQRPGQSPKRLIYLVSKLDSGVPDRFTGSGSGTDFTLKISRVEAEDLGVYHCWQGTHFPYTFGGGTKLEIKRADAAPTVSX

SEQ ID NO: 16 - DNA, 1413 bp, Artificial Sequence
  Heavy chain H1 (isotype IgG2a)
aagcttgccgccaccatgggatggtcttgtattattctgtttctggtcgccaccgccacaggagtgcattccgaagtccagctgaagcagtccggccccgaactggtcaagactggcgccagtgtgaaaatctcatgcaaggctagcgggtactctttcaccggttactatattcactgggtgaaacagtcccatggcaagagcctggaatggatcggatacatttcttgttataacggggcatccagctacaatcagaagttcaaaggcaaggccacctttacagtggacacctctagttcaacagcttatatgcagtttaacagtctgacatcaggcgactccgctgtgtactattgcgcatccagcatggattactgggggcagggtacatccgtcactgtgtctagtgcaaagaccacagcccccagcgtctatcctctggctccagtgtgcggcgatactaccggatcatccgtcactctgggctgtctggtgaagggatacttccctgagccagtgactctgacctggaactccgggagcctgagctctggtgtccacacctttcctgccgtgctgcagtctgacctgtatacactgagttcatccgtcacagtgactagctctacatggccttctcagagtatcacttgcaacgtggcccatccagctagttcaacaaaggtggataagaaaatcgaaccccggggccctaccatcaagccatgtcccccttgcaagtgtcccgctcctaatctgctgggcggaccctccgtgttcatctttccacccaaaattaaggacgtgctgatgatctcactgtcccccattgtcacctgtgtggtcgtggacgtgtctgaggacgatcctgatgtccagatctcctggttcgtgaacaatgtcgaagtgcacaccgctcagacccagacacatagggaggattacaactccacactgcgggtcgtgagcgcactgccaattcagcaccaggactggatgtccggaaaagagttcaagtgcaaggtgaacaataaggatctgccagcacccatcgagcgaaccatttctaaaccaaaggggagtgtgcgtgccccccaggtctatgtgctgcctccacccgaggaagagatgactaagaaacaggtcactctgacctgtatggtgaccgacttcatgcctgaagatatctacgtggagtggactaacaatggaaaaaccgaactgaactataagaataccgagccagtgctggacagcgatgggtcttactttatgtatagcaagctgagagtcgaaaagaaaaactgggtggagcgcaatagctactcttgcagtgtcgtgcacgagggtctgcataatcaccatacaactaaatcattctcccgcacacccggcaagtaatgagaattc

SEQ ID NO: 17 - protein, 462 aa, Artificial Sequence
  Heavy chain H1 (isotype IgG2a)
MGWSCIILFLVATATGVHSEVQLKQSGPELVKTGASVKISCKASGYSFTGYYIHWVKQSHGKSLEWIGYISCYNGASSYNQKFKGKATFTVDTSSSTAYMQFNSLTSGDSAVYYCASSMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 18 - DNA, 1422 bp, Artificial Sequence
  Heavy chain H2 (isotype IgG2a)
aagcttgccgccaccatgggttggtcttgtatcattctgtttctggtcgctaccgctactggggtccattccgatgtgcagctgaaactgcaggagtctgggccagggctggtgaagaacagtcagtcactgtccctgacctgcacagtgactggttatagcatcacttctgactacgcctggaactggattagacagttccccggcaataagctggaatggatggggtatatcagctactctggtagtacctcatataaccctagtctgaagtcaaggatctccattacccgggatacatctaaaaaccagttctttctgcagctgaactccgtgacacctgaggacaccgctacatactattgtgcacgcgatcccccttactattacgggagcaatggtactctgaccgtgtccagcgcaaagaccacagccccatctgtctatcccctggctcctgtgtgcggcgacactaccggatctagtgtcaccctggggtgtctggtgaagggttacttccccgagcctgtgacactgacttggaactccggcagcctgtcatccggagtccacacctttcccgcagtgctgcagtccgacctgtacacactgagctctagtgtcaccgtgacatcatccacatggccctctcagagtattacttgcaacgtcgcccatcctgctagctctacaaaggtggataagaaaatcgaaccacgaggccccactattaagccttgtccaccctgcaaatgtccagctcccaatctgctgggcggaccaagcgtgttcatctttcctccaaagatcaaggacgtgctgatgatctcactgtccccaattgtcacctgcgtggtcgtggacgtgtctgaggacgatcccgatgtccagatcagttggttcgtgaacaatgtcgaagtgcacaccgcacagactcagacccatagagaggattataactccacactgcgagtcgtgagcgcactgcctattcagcaccaggactggatgtctgggaaggagttcaagtgcaaagtgaacaacaaggatctgcctgccccaatcgagaggaccattagtaagcctaaaggatcagtgcgggctccacaggtctacgtgctgccacctccagaggaagagatgactaagaaacaggtcacactgacttgtatggtgaccgacttcatgccagaagatatctatgtggagtggactaacaatggcaagaccgaactgaactacaaaaatacagagcccgtgctggacagcgatggatcttattttatgtacagcaagctgcgagtcgaaaagaaaaactgggtggagcgtaatagctactcttgtagtgtcgtgcacgagggcctgcataatcaccatacaactaagtcattttcccggactcccggaaaataatgagaattc

SEQ ID NO: 19 - protein, 465 aa, Artificial Sequence
  Heavy chain H2 (isotype IgG2a)
MGWSCIILFLVATATGVHSDVQLKLQESGPGLVKNSQSLSLTCTVTGYSITSDYAWNWIRQFPGNKLEWMGYISYSGSTSYNPSLKSRISITRDTSKNQFFLQLNSVTPEDTATYYCARDPPYYYGSNGTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - DNA, 738 bp, Artificial Sequence
  Light chain L1
aagcttgccgccaccatgggctggtcctgtattatcctgttcctggtcgctactgctactggggtccattccgatgtcgtgatgactcagactccactgactctgtccgtgacaatcgggcagcccgccagcatttcttgcaagtccagccagtccctgctggacagcgatggcaaaacctacctgaactggctgctgcagaggccaggacagagccccaagcggctgatctatctggtgtctaaactggacagtggcgtccctgatagattcaccggaagtgggtcaggtactgactttaccctgaagatttctcgcgtggaggctgaagatctgggggtctaccactgctggcagggtacccatttcccttatacatttggcggagggactaagctggagatcaaacgggctgacgccgctccaactgtgtccattttccccccttctagtgaacagctgacctcaggtggcgcatccgtggtctgtttcctgaacaatttttacccaaaggacatcaacgtgaagtggaaaattgatggcagcgagcgccagaacggagtgctgaactcctggaccgaccaggattctaaggacagtacatattcaatgtcatccaccctgacactgactaaagatgagtacgaacgacacaatagttatacatgtgaagcaactcataagacctccacaagccccatcgtgaaatcctttaaccgtaatgcctaatgagaattc

SEQ ID NO: 21 - protein, 237 aa, Artificial Sequence
  Light chain L1
MGWSCIILFLVATATGVHSDVVMTQTPLTLSVTIGQPASISCKSSQSLLDSDGKTYLNWLLQRPGQSPKRLIYLVSKLDSGVPDRFTGSGSGTDFTLKISRVEAEDLGVYHCWQGTHFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNA

SEQ ID NO: 22 - DNA, 20 bp, Artificial Sequence
  Forward primer A
gatgtgaagcttcaggagtc

SEQ ID NO: 23 - DNA, 20 bp, Artificial Sequence
  Forward primer B
caggtgcagctgaaggagtc

SEQ ID NO: 24 - DNA, 36 bp, Artificial Sequence
  Reverse primer M
ggccagtggatagtcagatgggggtgtcgttttggc

SEQ ID NO: 25 - DNA, 20 bp, Artificial Sequence
  Forward primer C
caggtgcagctgaagcagtc

SEQ ID NO: 26 - DNA, 21 bp, Artificial Sequence
  Forward primer E
gaggtgcagctgcaacaatct

SEQ ID NO: 27 - DNA, 20 bp, Artificial Sequence
  Forward primer F
gaggtccagctgcagcagtc

SEQ ID NO: 28 - DNA, 21 bp, Artificial Sequence
  Forward primer G
caggtccaactgcagcagcct

SEQ ID NO: 29 - DNA, 20 bp, Artificial Sequence
  Forward primer L
gaggtgcagctggaggagtc

SEQ ID NO: 30 - DNA, 36 bp, Artificial Sequence; identical to SEQ ID NO: 24
  Reverse primer M

SEQ ID NO: 31 - DNA, 21 bp, Artificial Sequence
  Forward primer N
gatgttttgatgacccaaact

SEQ ID NO: 32 - DNA, 21 bp, Artificial Sequence
  Forward primer R
gacattgtgatgacccagtct

SEQ ID NO: 33 - DNA, 21 bp, Artificial Sequence
  Forward primer T
gatatccagatgacacagact

SEQ ID NO: 34 - DNA, 21 bp, Artificial Sequence
  Reverse primer X
ggatacagttggtgcagcatc

SEQ ID NO: 35 - protein, 45 aa, Homo sapiens
TRRMMRTKMRMRRMRRTRRKMRRKMSPARPRTSCREACLQGWTEA

SEQ ID NO: 36 - protein, 46 aa, Homo sapiens
NCRRMMRTKMRMRRMRRTRRKMRRKMSPARPRTSCREACLQGWTEA

SEQ ID NO: 37 - protein, 47 aa, Homo sapiens
QRTRRMMRTKMRMRRMRRTRRKMRRKMSPARPRTSCREACLQGWTEA

SEQ ID NO: 38 - protein, 50 aa, Homo sapiens
RRRQRTRRMMRTKMRMRRMRRTRRKMRRKMSPARPRTSCREACLQGWTEA

SEQ ID NO: 39 - protein, 48 aa, Homo sapiens
GQRTRRMMRTKMRMRRMRRTRRKMRRKMSPARPRTSCREACLQGWTEA

SEQ ID NO: 40 - protein, 49 aa, Homo sapiens
RRQRTRRMMRTKMRMRRMRRTRRKMRRKMSPARPRTSCREACLQGWTEA

SEQ ID NO: 41 - protein, 44 aa, Homo sapiens
RRMMRTKMRMRRMRRTRRKMRRKMSPARPRTSCREACLQGWTEA

SEQ ID NO: 42 - protein, 49 aa, Homo sapiens; identical to SEQ ID NO: 40

SEQ ID NO: 43 - protein, 48 aa, Homo sapiens
RQRTRRMMRTKMRMRRMRRTRRKMRRKMSPARPRTSCREACLQGWTEA

SEQ ID NO: 44 - protein, 46 aa, Homo sapiens
MCRRMMRTKMRMRRMRRTRRKMRRKMSPARPRTSCREACLQGWTEA

SEQ ID NO: 45 - protein, 50 aa, Homo sapiens
DQRQRTRRMMRTKMRMRRMRRTRRKMRRKMSPARPRTSCREACLQGWTEA

SEQ ID NO: 46 - protein, 51 aa, Homo sapiens
RRRRQRTRRMMRTKMRMRRMRRTRRKMRRKMSPARPRTSCREACLQGWTEA

SEQ ID NO: 47 - protein, 51 aa, Homo sapiens
QRRRQRTRRMMRTKMRMRRMRRTRRKMRRKMSPARPRTSCREACLQGWTEA

SEQ ID NO: 48 - protein, 50 aa, Homo sapiens; identical to SEQ ID NO: 38

SEQ ID NO: 49 - protein, 50 aa, Homo sapiens
RRRERTRRMMRTKMRMRRMRRTRRKMRRKMSPARPRTSCREACLQGWTEA

SEQ ID NO: 50 - protein, 50 aa, Homo sapiens
QRRQRTRRMMRTKMRMRRMRRTRRKMRRKMSPARPRTSCREACLQGWTEA

SEQ ID NO: 51 - protein, 49 aa, Homo sapiens
RRQWTRRMMRTKMRMRRMRRTRRKMRRKMSPARPRTSCREACLQGWTEA

SEQ ID NO: 52 - protein, 43 aa, Homo sapiens
RMMRTKMRMRRMRRTRRKMRRKMSPARPRTSCREACLQGWTEA

SEQ ID NO: 53 - protein, 48 aa, Homo sapiens; identical to SEQ ID NO: 43

SEQ ID NO: 54 - protein, 50 aa, Homo sapiens
GRRQRTRRMMRTKMRMRRMRRTRRKMRRKMSPARPRTSCREACLQGWTEA

SEQ ID NO: 55 - protein, 49 aa, Homo sapiens
AFKRTRRMMRTKMRMRRMRRTRRKMRRKMSPARPRTSCREACLQGWTEA

SEQ ID NO: 56 - protein, 54 aa, Homo sapiens
NAKRRRRQRTRRMMRTKMRMRRMRRTRRKMRRKMSPARPRTSCREACLQGWTEA

SEQ ID NO: 57 - protein, 53 aa, Homo sapiens
CVRRRRQRTRRMMRTKMRMRRMRRTRRKMRRKMSPARPRTSCREACLQGWTEA

SEQ ID NO: 58 - protein, 49 aa, Homo sapiens; identical to SEQ ID NO: 40

SEQ ID NO: 59 - protein, 48 aa, Homo sapiens; identical to SEQ ID NO: 43

SEQ ID NO: 60 - protein, 54 aa, Homo sapiens; identical to SEQ ID NO: 56

SEQ ID NO: 61 - protein, 55 aa, Homo sapiens
CFAKRRRRQRTRRMMRTKMRMRRMRRTRRKMRRKMSPARPRTSCREACLQGWTEA

SEQ ID NO: 62 - protein, 44 aa, Homo sapiens; identical to SEQ ID NO: 41

SEQ ID NO: 63 - protein, 49 aa, Homo sapiens
PPLCLRRMMRTKMRMRRMRRTRRKMRRKMSPARPRTSCREACLQGWTEA

SEQ ID NO: 64 - protein, 48 aa, Homo sapiens
DHPCRRMMRTKMRMRRMRRTRRKMRRKMSPARPRTSCREACLQGWTEA

SEQ ID NO: 65 - protein, 47 aa, Homo sapiens
GNCRRMMRTKMRMRRMRRTRRKMRRKMSPARPRTSCREACLQGWTEA

SEQ ID NO: 66 - protein, 45 aa, Homo sapiens
CRRMMRTKMRMRRMRRTRRKMRRKMSPARPRTSCREACLQGWTEA

SEQ ID NO: 67 - protein, 45 aa, Homo sapiens; identical to SEQ ID NO: 66

SEQ ID NO: 68 - protein, 46 aa, Homo sapiens
TCRRMMRTKMRMRRMRRTRRKMRRKMSPARPRTSCREACLQGWTEA

SEQ ID NO: 69 - protein, 46 aa, Homo sapiens
ICRRMMRTKMRMRRMRRTRRKMRRKMSPARPRTSCREACLQGWTEA

SEQ ID NO: 70 - protein, 45 aa, Homo sapiens; identical to SEQ ID NO: 66

SEQ ID NO: 71 - protein, 24 aa, Artificial Sequence
  synthetic peptide of C-terminal amino acid sequence of mutant calreticulin protein
RRKMSPARPRTSCREACLQGWTEA

SEQ ID NO: 72 - protein, 4 aa, Artificial Sequence
  last 4 amino acids of wildtype calreticulin
KDEL

SEQ ID NO: 73 - protein, 19 aa, Artificial Sequence
  inhibitory peptide
  VARIANT 15: Tyr = Phosphotyrosine
YARAAARQARAGRGYVSTT

SEQ ID NO: 74 - protein, 6 aa, Artificial Sequence
  inhibitory peptide
  VARIANT 2: Tyr = Phosphotyrosine
PYLKTK

SEQ ID NO: 75 - protein, 22 aa, Artificial Sequence
  inhibitory peptide
MVRRFLVTLRIRRACGPPRVRV

SEQ ID NO: 76 - DNA, 17 bp, Homo sapiens
gcagcagagaaacaaat

SEQ ID NO: 77 - DNA, 15 bp, Homo sapiens
aaggacgagctgtag

SEQ ID NO: 78 - protein, 5 aa, Homo sapiens
AAEKQ

SEQ ID NO: 79 - protein, 5 aa, Homo sapiens
QQRNK

SEQ ID NO: 80 - protein, 4 aa, Homo sapiens
RTSC

SEQ ID NO: 81 - protein, 5 aa, Homo sapiens
SRETN

SEQ ID NO: 82 - protein, 4 aa, Homo sapiens
GRAV

SEQ ID NO: 83 - DNA, 10 bp, Homo sapiens
aggaggaggg

SEQ ID NO: 84 - DNA, 11 bp, Homo sapiens
gaggaggcagt

SEQ ID NO: 85 - DNA, 14 bp, Homo sapiens
cctcctctttgtct

SEQ ID NO: 86 - DNA, 11 bp, Homo sapiens
ccatccttgtc